(19) United States
(12) Patent Application Publication
(10) Pub. No.: US 2022/0409727 A1
(43) Pub. Date: Dec. 29, 2022

(54) Metal Complexes Bearing Bisstyryl-Bipyridine Ligand and Their Use as Photosensitizer Agent in One and Two-Photon Photodynamic Therapy

(71) Applicants: Paris Sciences et Lettres, Paris (FR); Ecole Nationale Superieure de Chimie de Paris, Paris (FR)

(72) Inventors: Gilles Albert Gasser, Paris (FR); Johannes Karges, Eichenzell (DE)

(21) Appl. No.: 17/773,861

(22) PCT Filed: Nov. 4, 2020
(86) PCT No.: PCT/EP2020/081000

§ 371 Date: May 3, 2022

(30) Foreign Application Priority Data
Nov. 4, 2019 (EP) 19306429.2

(51) Int. Cl. A61K 41/00; C07F 15/00; A61P 35/00

(52) U.S. Cl. CPC A61K 41/008; C07F 15/0053; C07F 15/0026; A61P 35/00

(57) ABSTRACT

The present invention relates to metal complexes bearing at least one (E-E′)-4,4′-bisstyryl-2,2′-bipyridine ligand (LIG1) of the following formula (I): or a pharmaceutically acceptable salt and/or solvate thereof. The present invention also relates to pharmaceutical compositions comprising these complexes and at least one pharmaceutically acceptable excipient. The present invention also relates to the use of compounds of formula (I) or pharmaceutical compositions comprising thereof as drug and as photosensitizer agent in photodynamic therapy. The present invention relates to methods of preparation of said complexes.

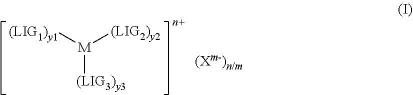

### FIELD OF THE INVENTION

The present invention relates to metal complexes bearing at least one (E-E′)-4,4′-bisstyryl-2,2′-bipyridine ligand, to pharmaceutical compositions comprising these complexes and to their use as drug and as photosensitizer agent in photodynamic therapy. The present invention also relates to methods of preparation of said complexes.

### BACKGROUND OF THE INVENTION

Photodynamic Therapy (PDT) is a non-invasive medical technique for the treatment of various types of cancer (i.e. lung, bladder, oesophageal and brain cancer) as well as bacterial, fungal or viral infections. The effect of PDT relies on the combination of an ideally non-toxic molecule, so called photosensitizer (PS), oxygen and light.

Photofrin is currently the most commonly used PS in PDT. It has been approved for the treatment of bladder cancer, early stage lung cancer, oesophageal cancer and early non-small cell lung cancer. However, based on its low solubility and low absorption at the therapeutic wavelengths, high concentrations as well as high light doses required for an adequate tumor treatment. Photofrin is not an ideal PS. Additionally, it was shown that the drug has an exceptionally long half-life excretion time, leading to severe photosensitivity for the patients. Therefore, the application of the approved PSs is currently limited by their poor aqueous solubility, aggregation, photobleaching, slow clearance from the body and hepatotoxicity.

New classes of PSs are thus being developed by the scientists. Among these new classes of PSs, Ru(II) polypyridine complexes have gained increasing attention due to their attractive chemical and photophysical properties (McFarland, S. A. et al., 2019 and Gasser, G. et al., 2017). However, these complexes lack significant absorption in the biological spectral window (600-900 nm), limiting their application due to poor tissue penetration. Indeed, the majority of the investigated Ru(II) polypyridine complexes require blue (400-450 nm) or UV-A light activation (315-400 nm), limiting their application in PDT. Since longer wavelengths in the biological spectral window (600-900 nm) are able to penetrate deeper inside the tissue, deeper-seated tumours or larger tumours could be treated. Additionally, longer wavelengths are less energetic and therefore less potential damaging (Gasser, G. et al., 2017, Gollnick, S. O. et al., 2011, Glazer, E. C. et al., 2012, and Ogawa, K. and Kobuke, Y., 2008).

There is thus a need for complexes which are able to absorb longer wavelengths in the biological spectral window in order to reach deeper-seated tumors.

The inventors have synthesized metal complexes bearing at least one (E-E′)-4,4′-bisstyryl-2,2′-bipyridine ligand which are able to be excited between 450-900 nm by either one- or two-photon absorption.

### SUMMARY OF THE INVENTION

The inventors have thus designed metal complexes bearing at least one (E-E′)-4,4′-bisstyryl-2,2′-bipyridine ligand useful as a photosensitizer in photodynamic therapy, in particular to treat deep-seated and/or large tumors.

In a first aspect, the present invention thus relates to a compound of the following formula (I):

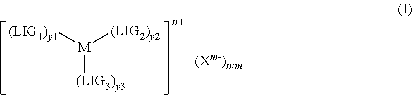

or a pharmaceutically acceptable salt and/or solvate thereof, wherein

M is selected among ruthenium, rhenium, osmium, rhodium, iridium and platinum, LIG₁ is a bidentate ligand having the following formula:

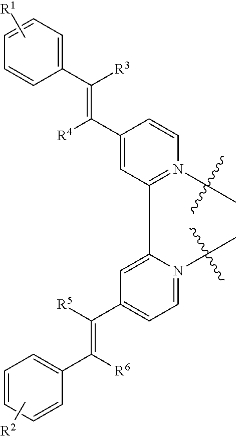

wherein

the wavy lines indicate the points of attachment to M,

R¹ and R² each independently represent one or several substituents selected in the group consisting of H, halogen, C₁-C₆ alkyl, C₆-C₁₀ aryl, OR⁹ and NR¹⁰R¹¹,

R³ to R⁶ each independently represent a substituent selected in the group consisting of H, halogen, C₁-C₆ alkyl, C₆-C₁₀ aryl, OR¹² and NR¹³R¹⁴,

R⁷ and R⁸ each independently represent one or several substituents selected in the group consisting of H, halogen, C₁-C₆ alkyl, C₆-C₁₀ aryl, OR¹⁵ and NR¹⁶R¹⁷,

R⁹ to R¹¹ are each independently selected in the group consisting of H and C₁-C₆ alkyl, and

R¹² to R¹⁷ are each independently selected in the group consisting of H, C₁-C₆ alkyl and CO—(C₁-C₆ alkyl),

LIG₂ is a bidentate ligand having the following formula (a) or (b):

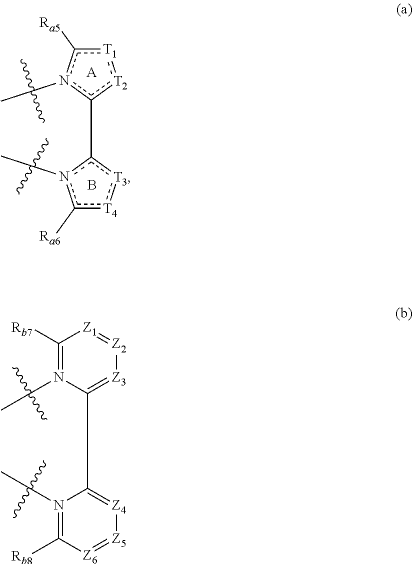

wherein the wavy lines indicate the points of attachment to M,

LIG₃ is a bidentate ligand having the following formula (c) or (d):

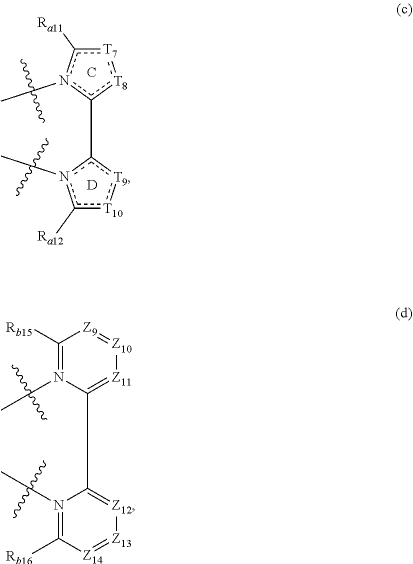

wherein the wavy lines indicate the points of attachment to M,

each [symbol] represents a single or a double bond, provided that each cycle A, b, C and D is a heteroaromatic cycle,

T₁ is NRₐ₁ (e.g. NH) or CRₐ₁, T₂ is NRₐ₂ (e.g. NH) or CRₐ₂, T₃ is NRₐ₃ (e.g. NH) or CRₐ₃, T₄ is NRₐ₄ (e.g. NH) or CRₐ₄, T₇ is NRₐ₇ (e.g. NH) or CRₐ₇, T₈ is NRₐ₈ (e.g. NH) or CRₐ₈, T₉ is NRₐ₉ (e.g. NH) or CRₐ₉ and T₁₀ is NRₐ₁₀ (e.g. NH) or CRₐ₁₀, provided that when T₁ is NRₐ₁₁, then T₂ is CRₐ₂, when T₃ is NRₐ₃, then T₄ is CRₐ₄, when T₇ is NRₐ₇, then T₈ is CRₐ₈ and when T₉ is NRₐ₉, then T₁₀ is CRₐ₁₀,

Z₁ is N or CR_{b1}, Z₂ is N or CR_{b2}, Z₃ is N or CR_{b3}, Z₄ is N or CR_{b4}, Z₅ is N or CR_{b5}, Z₆ is N or CR_{b6}, Z₉ is N or CR_{b9}, Z₁₀ is N or CR_{b10}, Z₁₁ is N or CR_{b11}, Z₁₂ is N or CR_{b12}, Z₁₃ is N or CR_{b13} and Z₁₄ is N or CR_{b14}, provided that at least two of Z₁ to Z₃ and at least two of Z₄ to Z₆ and at least two of Z₉ to Z₁₁ and at least two of Z₂ to Z₁₄ are not N, Rₐ₁ to Rₐ₁₂ and R_{b1} to R_{b16} each independently represent H, halogen, optionally substituted C₁-C₆ alkyl, optionally substituted C₂-C₆ alkenyl, optionally substituted C₂-C₆ alkynyl, optionally substituted carbocycle, optionally substituted aryl, optionally substituted heteroaryl, optionally substituted heterocycle, CN, NO₂, N₃, COR¹⁸, OR¹⁹ or NR²⁰R²¹,

or Z₃ and Z₄ in formula (b) are linked together so that LIG₂ represents:

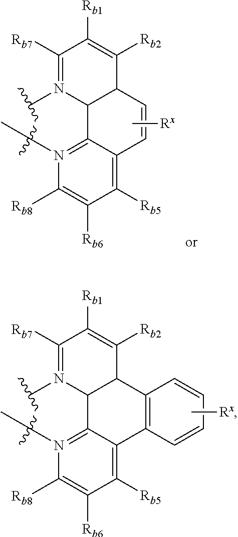

Z₁₁ and Z₁₂ are linked in formula (d) together so that LIG₃ represents:

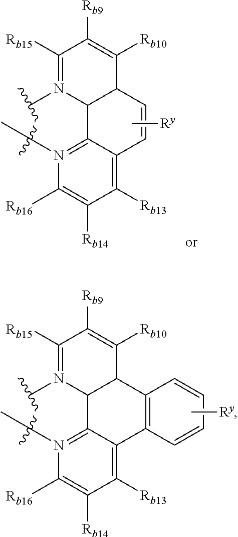

wherein RX and R^{Y} each independently represent one or several substituents selected in the group consisting of H, halogen, optionally substituted C₁-C₆ alkyl, optionally substituted C₂-C₆ alkenyl, optionally substituted C₂-C₆ alkynyl, optionally substituted carbocycle, optionally substituted aryl, optionally substituted heteroaryl, optionally substituted heterocycle, CN, NO₂, N₃, COR¹⁸, OR¹⁹ and NR²⁰R²¹,

R¹⁸ is selected in the group consisting of H, optionally substituted C₁-C₆ alkyl, OR²² and NR²³R²⁴,

R¹⁹ to R²⁴ are each independently selected in the group consisting of H, optionally substituted C₁-C₆ alkyl and optionally substituted CO—(C₁-C₆alkyl),

X^{m-} is a pharmaceutically acceptable anion, preferably selected in the group consisting of PF₆⁻, Cl⁻, Br⁻, I⁻, BF₄⁻, (C₁-C₆ alkyl)-C(O)O⁻, (C₁-C₆ haloalkyl)-C(O)O⁻, (C₁-C₆ alkyl)-SO₃⁻, (C₁-C₆-haloalkyl)-SO₃⁻, SO₄²⁻ and PO₄³⁻,

m and n are independently 1, 2, 3 or 4, wherein n is 1 when M is rhenium, n is 2 when

M is ruthenium or osmium, n is 3 when M is rhodium or iridium and n is 4 when M is platinum, and

y1 is 1, 2 or 3, y2 and y3 are independently 0, 1 or 2, provided that y1+y2+y3 is 3.

In a second aspect, the present invention relates to a pharmaceutical composition comprising at least one compound of formula (I) or a pharmaceutically acceptable salt and/or solvate thereof and at least one pharmaceutically acceptable excipient.

In a third aspect, the present invention relates to a compound of formula (I) according to the invention or a pharmaceutically acceptable salt and/or solvate thereof, or a pharmaceutical composition according to the present invention for use as a drug.

The present invention also relates to the use of a compound of formula (I) according to the invention or a pharmaceutically acceptable salt and/or solvate thereof, or a pharmaceutical composition according to the present invention as a drug or for the manufacture of a drug.

In a fourth aspect, the present invention relates to a compound of formula (I) according to the invention or a pharmaceutically acceptable salt and/or solvate thereof, or a pharmaceutical composition according to the present invention for use as a photosensitizer agent in photodynamic therapy.

The present invention therefore also relates to the use of a compound of formula (I) or a pharmaceutically acceptable salt and/or solvate thereof or a pharmaceutical composition as described herein for the manufacture of a drug intended to be used as a photosensitizer agent in photodynamic therapy.

The present invention also relates to the use of a compound of formula (I) or a pharmaceutically acceptable salt and/or solvate thereof or a pharmaceutical composition as described herein as a photosensitizer agent in photodynamic therapy.

The present invention also concerns a method of treatment by photodynamic therapy comprising administering to an animal, in particular a mammal such as a human, in need thereof an effective amount of a compound of formula (I) or a pharmaceutically acceptable salt and/or solvate thereof as a photosensitizer agent.

In a fifth aspect, the present invention concerns methods of preparation of compounds of formula (I) according to the invention.

Preferably, said compound is not:

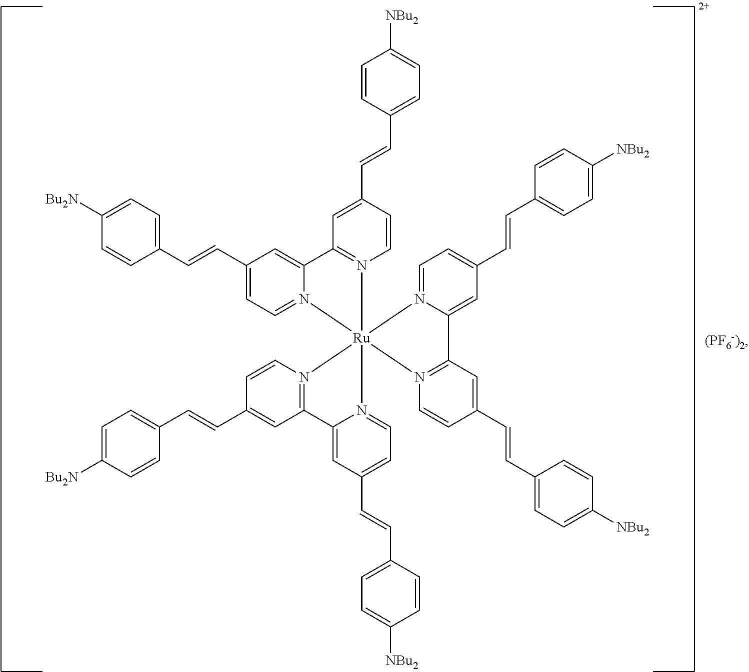

which is described in Humphrey, M. G. et al., 2007.

Preferably, said compound of formula (I) is also not:

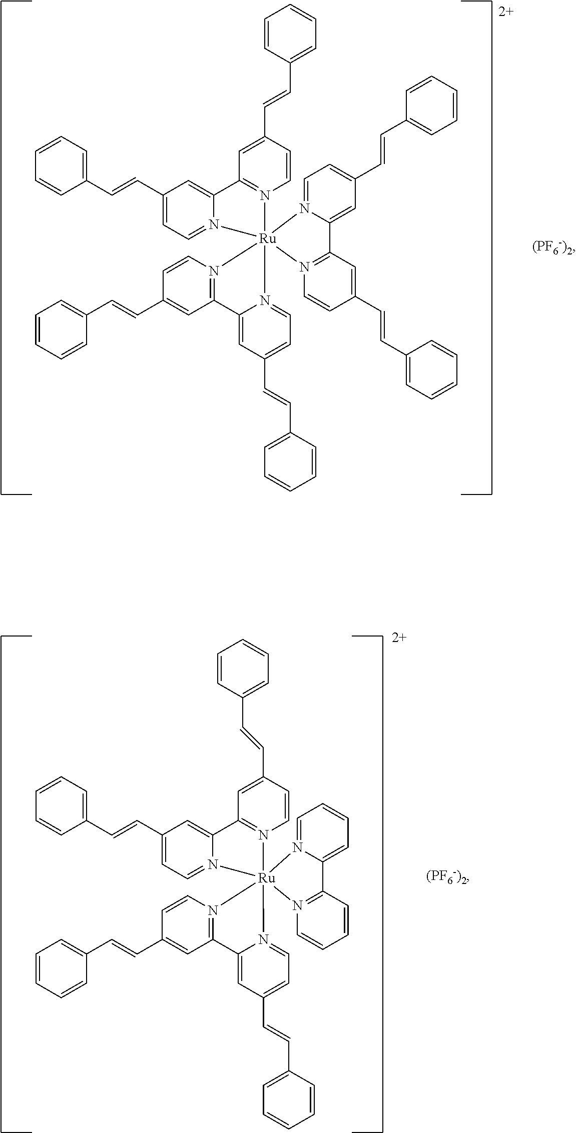

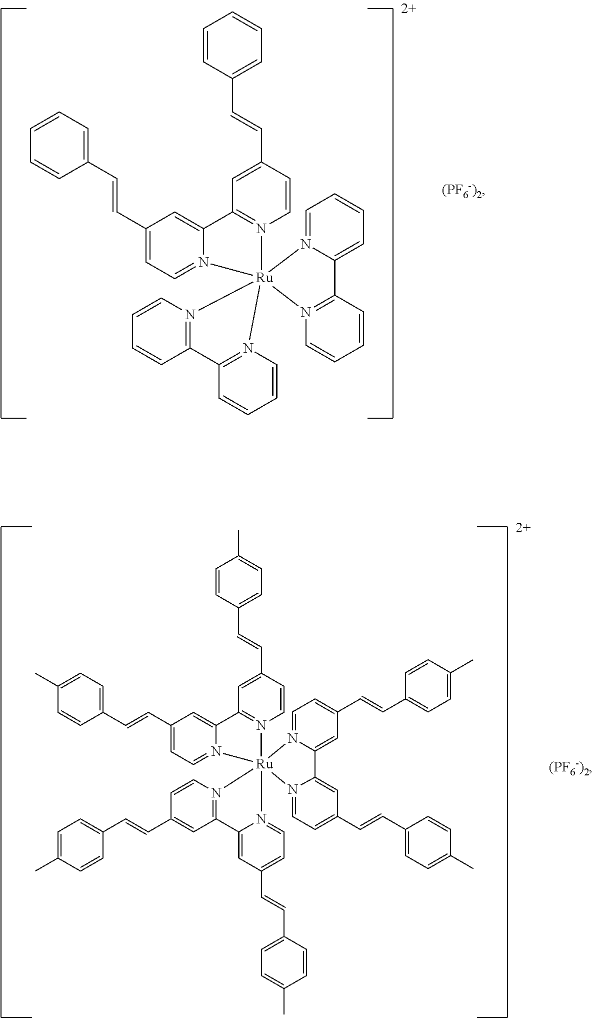

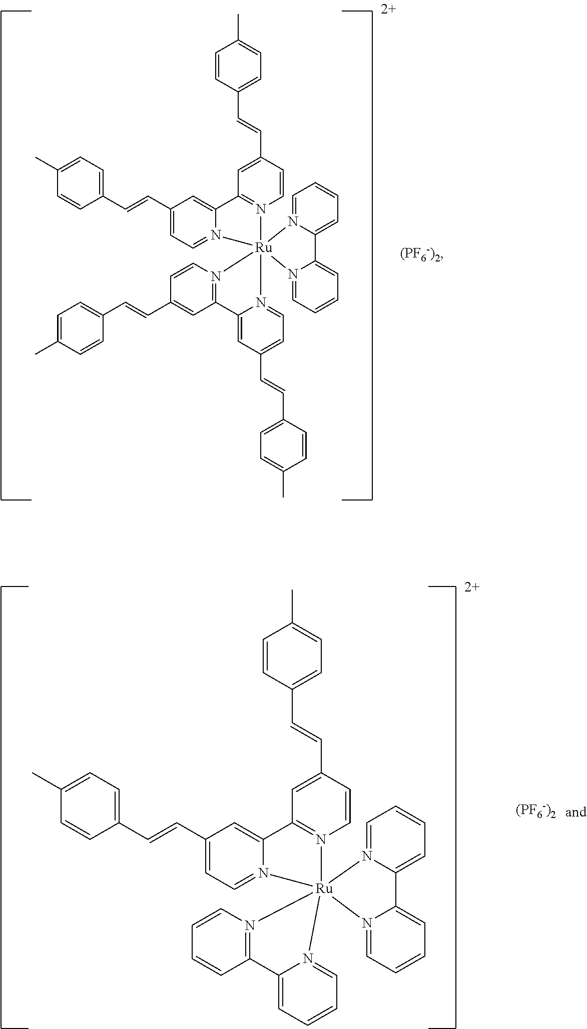

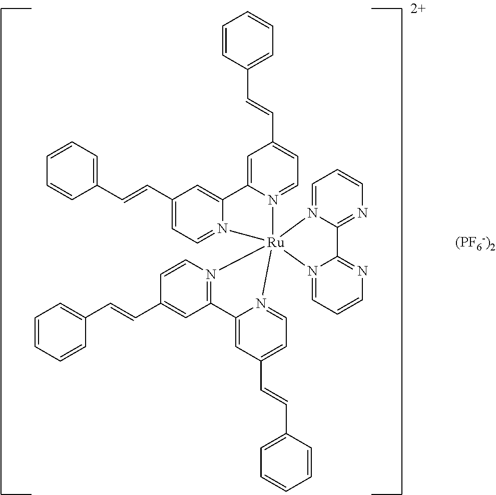

described in Leidner et al., 1987. Preferably, said compound of formula (I) is also not:

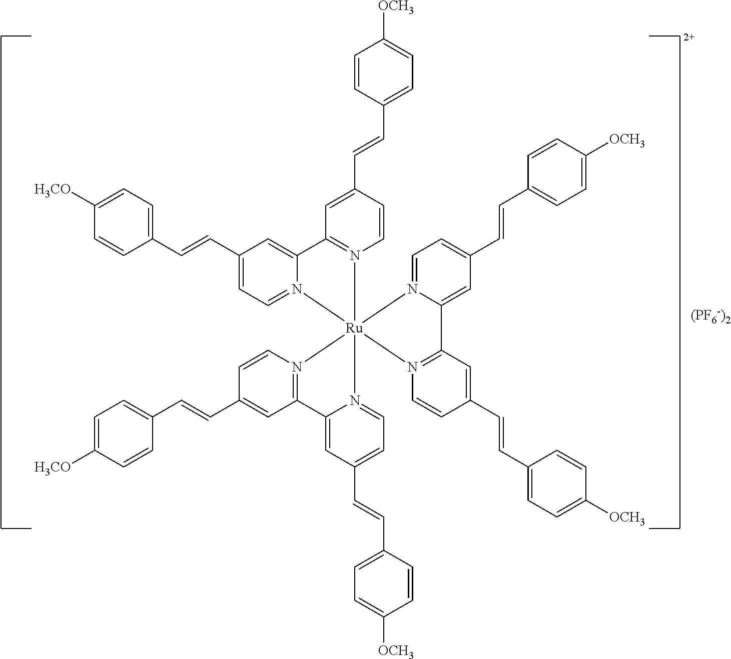

described in Zuniga César et al., 2014.

Preferably said compound of formula (I) is also not:

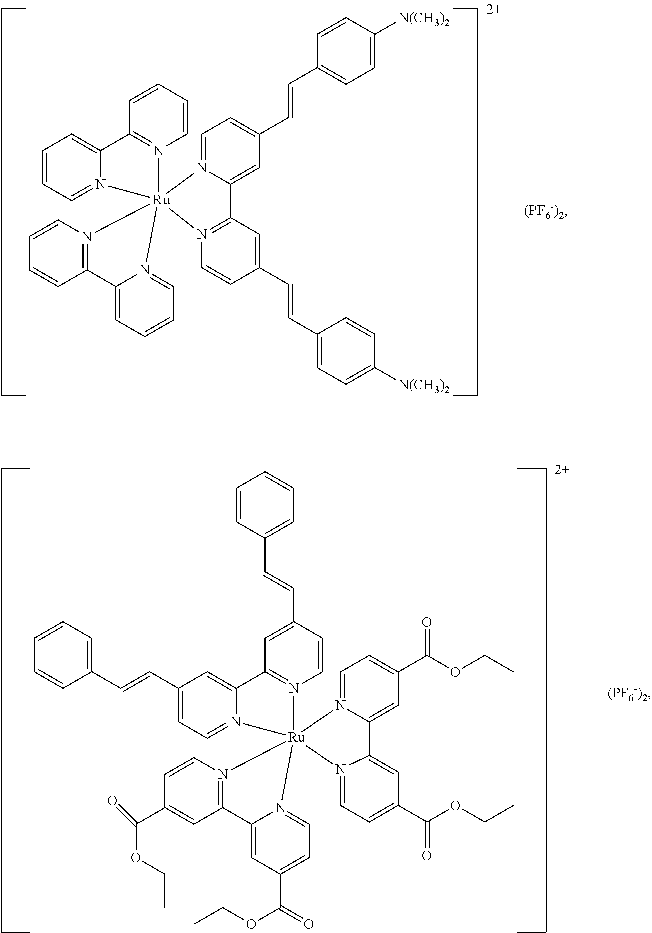

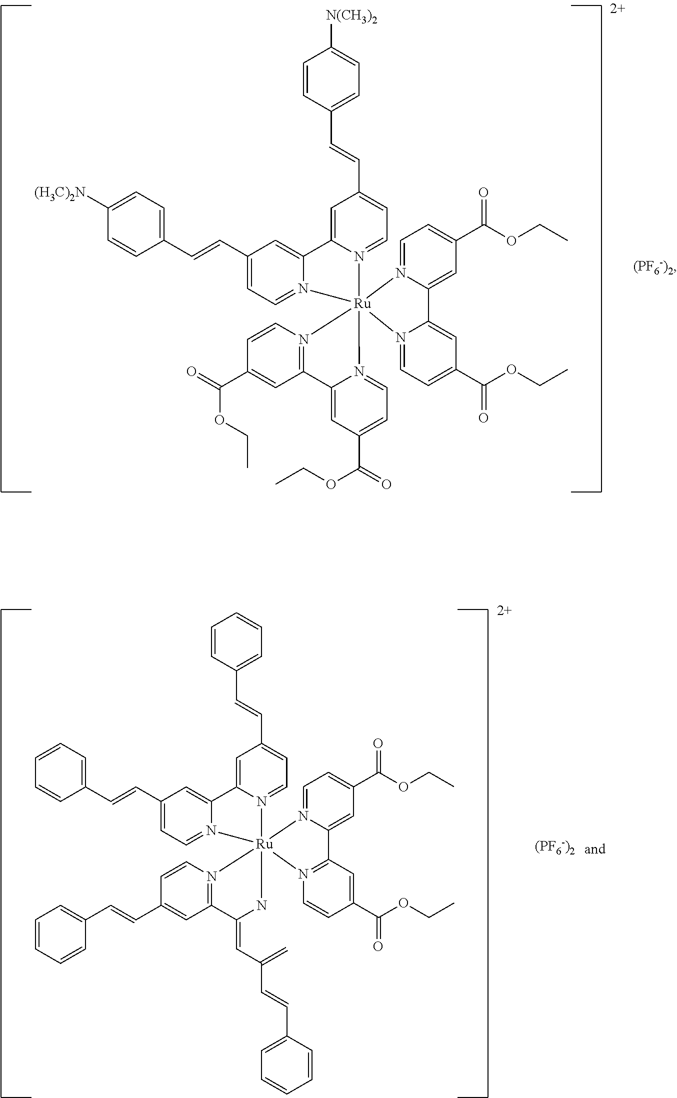

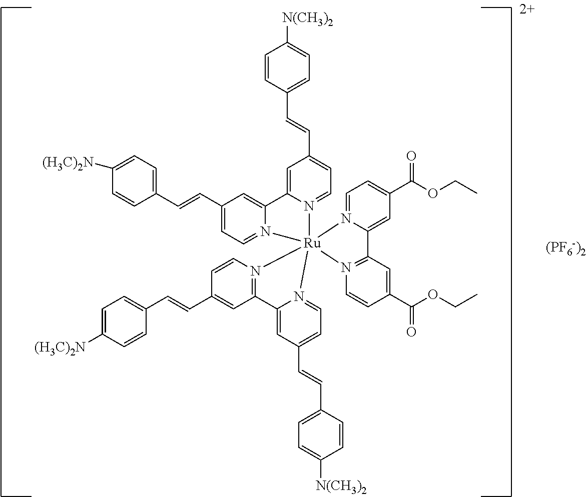

described in Francisco Gajardo et al., 2011.

Preferably said compound of formula (I) is also not:

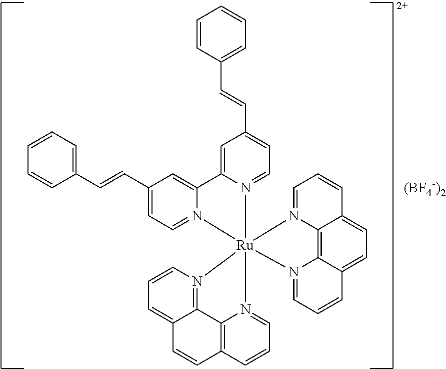

described in Dreyse, P. et al., 2013. Preferably, said compound is also not:

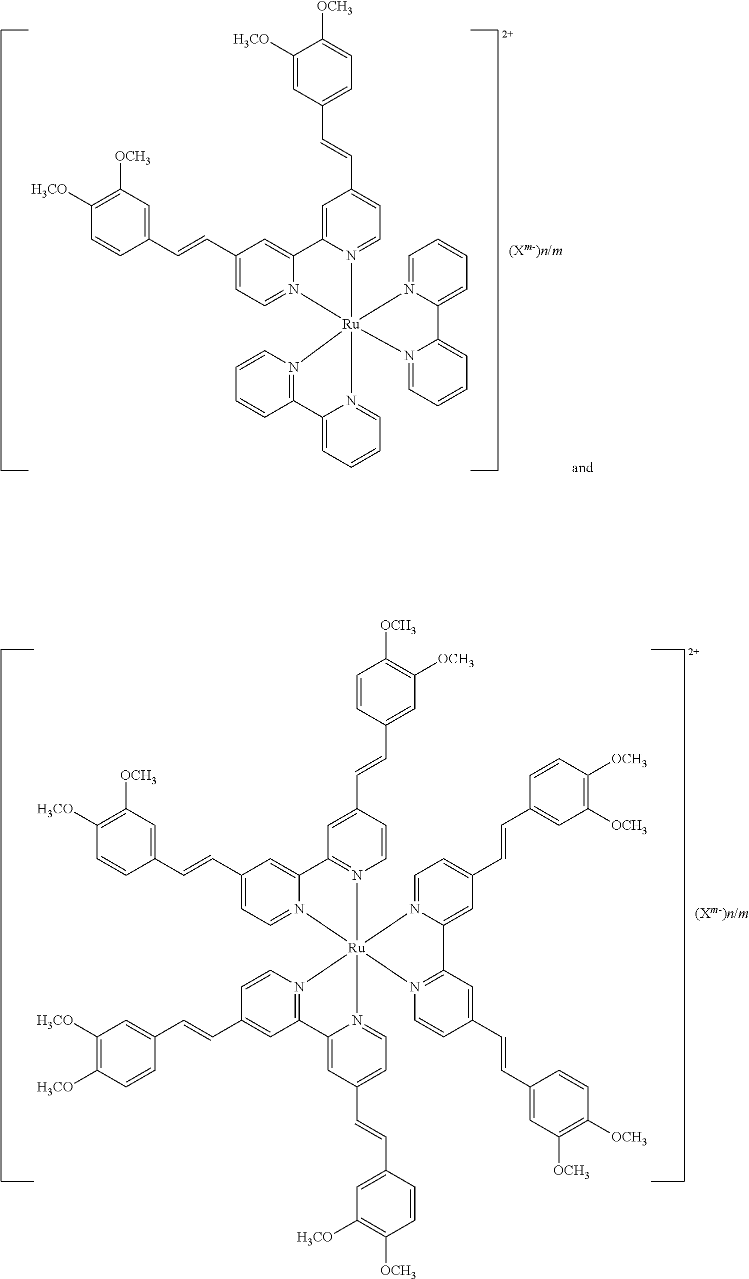

described in Ayman, A. et al., 2000.

### Definitions

The term “stereoisomers” used in this invention refers to configurational stereoisomers and more particularly to optical isomers.

In the present invention, the optical isomers result in particular from the different position in space of the three bidentate ligands of the metal. The metal of the complex thus represents a chiral or asymmetric center. Optical isomers that are not mirror images of one another are thus designated as “diastereoisomers”, and optical isomers, which are non-superimposable mirror images are designated as “enantiomers”.

An equimolar mixture of two enantiomers of a chiral compound is designated as a racemic mixture or racemate.

For the purpose of the invention, the term “pharmaceutically acceptable” is intended to mean what is useful to the preparation of a pharmaceutical composition, and what is generally safe and non-toxic, for a pharmaceutical use.

The term “pharmaceutically acceptable salt and/or solvate” is intended to mean, in the framework of the present invention, a salt and/or solvate of a compound which is pharmaceutically acceptable, as defined above, and which possesses the pharmacological activity of the corresponding compound.

The pharmaceutically acceptable salts comprise:

(1) acid addition salts formed with inorganic acids such as hydrochloric, hydrobromic, sulfuric, nitric and phosphoric acid and the like; or formed with organic acids such as acetic, benzenesulfonic, fumaric, glucoheptonic, gluconic, glutamic, glycolic, hydroxynaphtoic, 2-hydroxyethanesulfonic, lactic, maleic, malic, mandelic, methanesulfonic, muconic, 2-naphtalenesulfonic, propionic, succinic, dibenzoyl-L25 tartaric, tartaric, p-toluenesulfonic, trimethylacetic, and trifluoroacetic acid and the like, and

(2) base addition salts formed when an acid proton present in the compound is either replaced by a metal ion, such as an alkali metal ion, an alkaline-earth metal ion, or an aluminium ion; or coordinated with an organic or inorganic base. Acceptable organic bases comprise diethanolamine, ethanolamine, N-methylglucamine, triethanolamine, tromethamine and the like. Acceptable inorganic bases comprise aluminium hydroxide, calcium hydroxide, potassium hydroxide, sodium carbonate and sodium hydroxide. Acceptable solvates for the therapeutic use of the compounds of the present invention include conventional solvates such as those formed during the last step of the preparation of the compounds of the invention due to the presence of solvents. As an example, mention may be made of solvates due to the presence of water (these solvates are also called hydrates) or ethanol.

The term “halogen”, as used in the present invention, refers to a fluorine, bromine, chlorine or iodine atom.

The term “C₁-C₆ alkyl”, as used in the present invention, refers to a straight or branched monovalent saturated hydrocarbon chain containing from 1 to 6 carbon atoms including, but not limited to, methyl, ethyl, n-propyl, iso-propyl, n-butyl, iso-butyl, sec-butyl, t-butyl, n-pentyl, n-hexyl, and the like.

The term “C₂-C₆ alkenyl”, as used in the present invention, refers to a straight or branched monovalent unsaturated hydrocarbon chain containing from 2 to 6 carbon atoms and comprising at least one double bond including, but not limited to, ethenyl, propenyl, butenyl, pentenyl, hexenyl and the like.

The term “C₂-C₆ alkynyl”, as used in the present invention, refers to a straight or branched monovalent unsaturated hydrocarbon chain containing from 2 to 6 carbon atoms and comprising at least one triple bond including, but not limited to, ethynyl, propynyl, propynyl, butynyl, pentynyl, hexynyl and the like.

The term “C₁-C₆ haloalkyl” refers to a C₁-C₆ alkyl chain as defined above wherein one or more hydrogen atoms are replaced by a halogen atom selected from fluorine, chlorine, bromine or iodine, preferably a fluorine atom. For example, it is a CF₃ group.

The term “carbocycle” refers to a non-aromatic hydrocarbon ring, saturated or unsaturated, typically comprising from 3 to 20 carbons and comprising one or more fused or bridged ring(s). For example, it is a saturated hydrocarbon cycle, especially a C₃-C₇ cycloalkyl. In particular, it is an unsaturated hydrocarbon cycle, especially a C₃-C₈ cycloalkene or cycloalkyne including, but not limited to, cyclopropene, cyclobutene, cyclopentene, cyclohexene, 1,4-cyclohexadiene, cycloheptene, cycloheptyne, cyclooctene, cyclooctyne and the like.

The term “C₃-C₇ cycloalkyl” refers to a saturated hydrocarbon ring comprising from 3 to 7 carbons, including cyclopropyl, cyclobutyl, cyclopentyl, cyclohexyl and cycloheptyl. The term “heterocycle” as used in the present invention refers to a non-aromatic, saturated or unsaturated monocycle or polycycle (comprising fused, bridged or spiro rings) comprising preferably 5 to 10, notably 5 or 6, atoms in the ring(s), in which the atoms of the ring(s) consist of carbon atoms and one or more, advantageously 1 to 4, and more advantageously 1 or 2, heteroatoms, such as a nitrogen, oxygen or sulphur atom, the remainder being carbon atoms. In particular, it can be an unsaturated ring, such as an unsaturated 5 or 6-membered monocycle. Preferably it comprises 1 or 2 nitrogen, in particular one. A heterocycle can be notably piperidinyl, piperizinyl, pyrrolidinyl, pyrazolidinyl, imidazolidinyl, azepanyl, thiazolidinyl, isothiazolidinyl, oxazocanyl, thiazepanyl, benzimidazolonyl.

The term “aryl” refers to an aromatic hydrocarbon group preferably comprising from 6 to 12 carbon atoms and comprising one or more fused rings, such as, for example, a phenyl or naphthyl group. Advantageously, it is a phenyl group.

The term “heteroaryl”, as used in the present invention, refers to an aromatic group comprising one or several, notably one or two, fused hydrocarbon cycles in which one or several, notably one to four, advantageously one or two, carbon atoms each have been replaced with a heteroatom selected from a sulfur atom, an oxygen atom and a nitrogen atom, preferably selected from an oxygen atom and a nitrogen atom. It can be a furyl, thienyl, pyrrolyl, pyridyl, oxazolyl, isoxazolyl, thiazolyle, isothiazolyl, imidazolyl, pyrazolyl, oxadiazolyl, thiadiazolyl, triazolyl, tetrazolyl, pyridazinyl, pyrimidinyl, pyrazinyl, triazinyl, quinolyl, isoquinolyl, quinoxalyl or indyl.

In the context of the present invention, “unsaturated” means that the hydrocarbon chain may contain one or more unsaturation(s), i.e. a double bond C═C or a triple bond C≡C, advantageously one.

In the context of the present invention, “optionally substituted” means that the group in question is optionally substituted with one or more substituents which may be selected in particular from halogen, C₁-C₆ alkyl, C₁-C₆ haloalkyl, C₂-C₆ alkenyl, C₂-C₆ alkynyl, aryl, N₃, oxo, NR^{a}R^{b}, COR^{c}, CO₂R^{d}, CONR^{e}R^{f}, OR^{g}, CN and NO₂ wherein R^{a} to R^{g} are, independently of one another, H, C₁-C₆ alkyl, C₁-C₆ haloalkyl or aryl, preferably H or C₁-C₆ alkyl. In the context of the present invention, when a group is “optionally substituted”, it is preferably optionally substituted with one or more substituents selected from halogen, C₁-C₆ alkyl, aryl, NR^{a}R^{b} and OR^{g}, R^{a}, R^{b} and R^{g} being preferably H or C₁-C₆ alkyl.

The term “pharmaceutical composition” is meant in the framework of the present invention a composition having preventive and curative properties towards disease such as cancer, in particular lung cancer, bladder cancer, oesophageal cancer, colon cancer, stomach cancer, liver cancer, skin cancer, ovarian cancer, pancreatic cancer, head and neck cancer, or brain cancer; bacterial infection, such as sinusitis, diabetic feet, burned wounds; fungal infection, such as mycoses; viral infection such as herpes; and skin disorders, such as acne, port wine stains. In particular, the pharmaceutical composition in the context of the present invention has curative properties in photodynamic therapy, i.e. in combination with molecular oxygen and light irradiation. The term “photodynamic therapy” (PDT) refers to a non-invasive medical therapy which involves light and a photosensitizing chemical substance, called a photosensitizer (PS) used in conjunction with molecular oxygen to elicit cell death. The PDT is notably intended to treat a disease selected from cancer, bacterial infection, fungal infection, viral infection and skin disorders. A photosensitizer becomes highly toxic upon light irradiation, notably at wavelengths comprised between 450 nm and 900 nm.

During photodynamic therapy, the PS is administered either systemically or locally. The diseased area is then exposed to light. Upon light irradiation, the PS is able to create reactive oxygen species (ROS), such as singlet oxygen (¹O₂) or other radicals. Due to their high reactivity, these species can cause oxidative stress and damage in different surrounding cellular compartments (i.e. membrane, nucleus, endoplasmic reticulum, lysosome, mitochondria) leading to cell death.

### DETAILED DESCRIPTION

Compound of Formula (I)

The compounds according to the present invention can be in the form of a stereoisomer or a mixture of stereoisomers, such as a mixture of enantiomers, notably a racemic mixture, provided that LIG₁ is a (E,E′)-4-4′-bisstyryl-2,2′-bipyridine derivative. When y1 is different from 3, LIG₂ and LIG₃ are preferably different from LIG1.

According to a particular embodiment, y1 is 1, 2 or 3, y2 is 2, 1 or 0 and y3 is 0. In other terms, compound of formula (I) advantageously corresponds to the following formulas (I-A), (I-B) or (I-C):

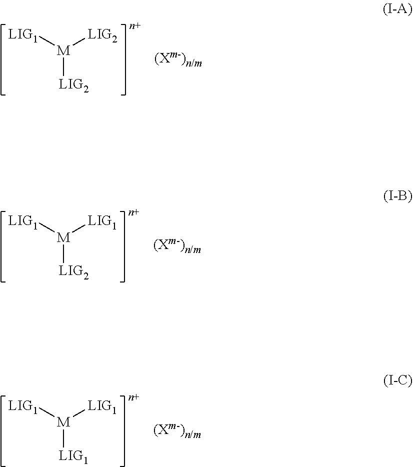

Compound of formula (I) is preferably (I-A) or (I-B), preferably (I-A). In other terms, y1 is advantageously 1 or 2, preferably 1, y2 is 2 or 1 respectively, preferably 2, and y3 is 0. Preferably, y1 is 1, y2 is 2 and y3 is 0. According to another particular embodiment, y1 is 2, y2 is 1 and y3 is 0.

It can be also a compound of following formula (I-D)

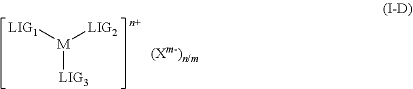

when y1=y2=y3=1.

According to a preferred embodiment, M is selected among ruthenium, osmium and iridium, preferably M is ruthenium or osmium, notably ruthenium.

LIG₁

LIG1 is a bidentate ligand having the following formula:

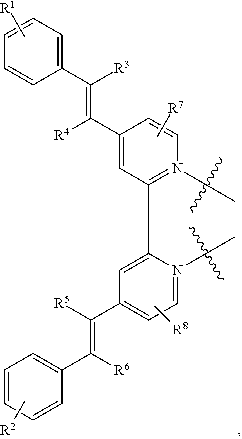

Advantageously, R¹ and R² each independently represent one or several substituents selected in the group consisting of H, halogen such as fluorine, OR⁹ and NR¹⁰R¹¹, wherein R⁹ to R¹¹ are preferably each independently H or C₁-C₆ alkyl, such as methyl.

Preferably, R¹ and R² each independently represent one or several substituents, notably one substituent, selected in the group consisting of halogen, such as fluorine, OR⁹ and NR¹⁰R¹¹, wherein R⁹ to R¹¹ are as defined above. More preferably, R¹ and R² each independently represent one or several substituents, notably one substituent, selected from OR⁹ and NR¹⁰R¹¹, in particular OR⁹, R⁹ to R¹¹ being preferably H or C₁-C₆ alkyl, in particular C₁-C₆ alkyl, such as methyl.

Advantageously, R¹ and R² each represent one substituent as defined above, and preferably OR⁹. According to this embodiment, R¹ and R² are in ortho, meta or para position of the phenyl group, notably in para position.

According to a preferred embodiment, R¹ and R² are identical.

Advantageously, R³ to R⁶ each independently represent a substituent selected in the group consisting of H, C₁-C₆ alkyl and C₆-C₁₀ aryl, preferably H and C₁-C₆ alkyl, such as methyl. More preferably, R³ and R⁶ are identical to each other and R⁴ and R⁵ are identical to each other. According to a particular embodiment, R³ and R⁶ are C₁-C₆ alkyl, in particular methyl and R⁴ and R⁵ are both hydrogen. According to another particular embodiment, R³ and R⁶ are both hydrogen and R⁴ and R⁵ are C₁-C₆ alkyl, in particular methyl. Even more preferably R³ to R⁶ are hydrogen.

Advantageously, R⁷ and R⁸ each independently represent one or several substituents, preferably one, selected in the group consisting of H and C₁-C₆ alkyl, such as methyl. Typically, R⁷ and R⁸ are identical. Preferably R⁷ and R⁸ are hydrogen.

In a preferred embodiment, LIG₁ is of formula (LIG1-A):

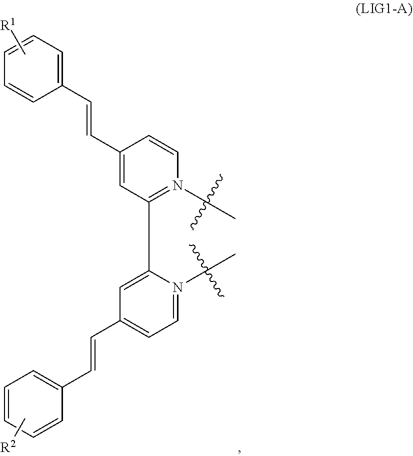

wherein R¹ and R² are as defined above and preferably R¹ and R² each represent one OR⁹ group.

Advantageously, when R¹ and R² are both NR¹⁰R¹¹ in LIG₁, y1 is 1 or 2 and LIG₂ and LIG₃ are different from LIG₁.

LIG₂

In a particular embodiment, LIG₂ is a bidentate ligand of the following formula (a):

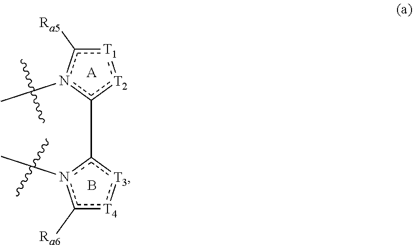

In particular, LIG₂ of formula (a) may correspond to the following ligands:

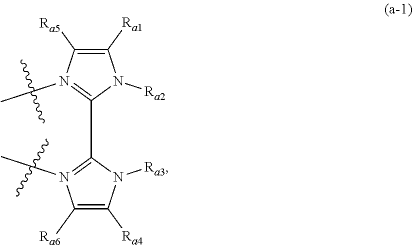

in particular

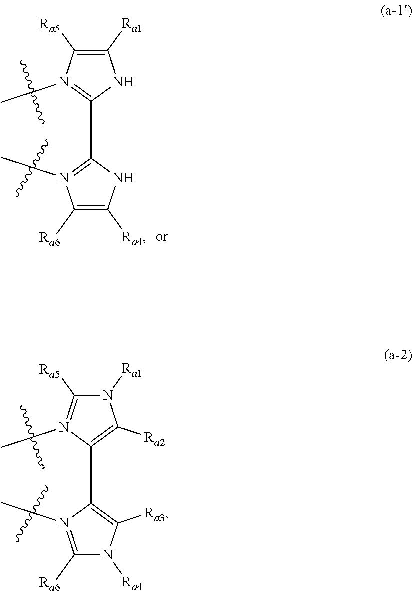

in particular

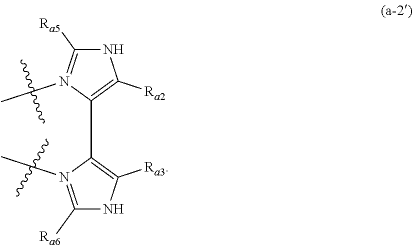

Advantageously, LIG₂ of formula (a) corresponds to formula (a-1), notably (a-1′). Preferably, when LIG₂ is of formula (a), in particular of formula (a-1), notably (a-1′), Rₐ₁ to Rae each independently represent H, halogen, optionally substituted C₁-C₆ alkyl, optionally substituted aryl, OR¹⁹ or NR²⁰R²¹, with R¹⁹ to R²¹ being preferably each independently selected in the group consisting of H and C₁-C₆ alkyl. More preferably, Rₐ₁ to Rae each independently represent H, halogen, C₁-C₆ alkyl, aryl, OR¹⁹ or NR²⁰R²¹, with R¹⁹ to R²¹ being as defined above such as H or C₁-C₆ alkyl. Even more preferably, Rₐ₁ to Rae each independently represent H or C₁-C₆ alkyl. Typically, Rₐ₁ to Rae are identical. In a preferred embodiment, Rₐ₁ to Rₐ₆ are H.

In a preferred embodiment, LIG₂ is a bidentate ligand of the following formula (b):

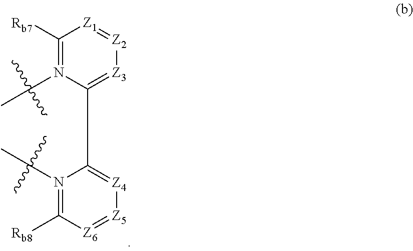

In particular, LIG₂ of formula (b) may correspond to the following ligands:

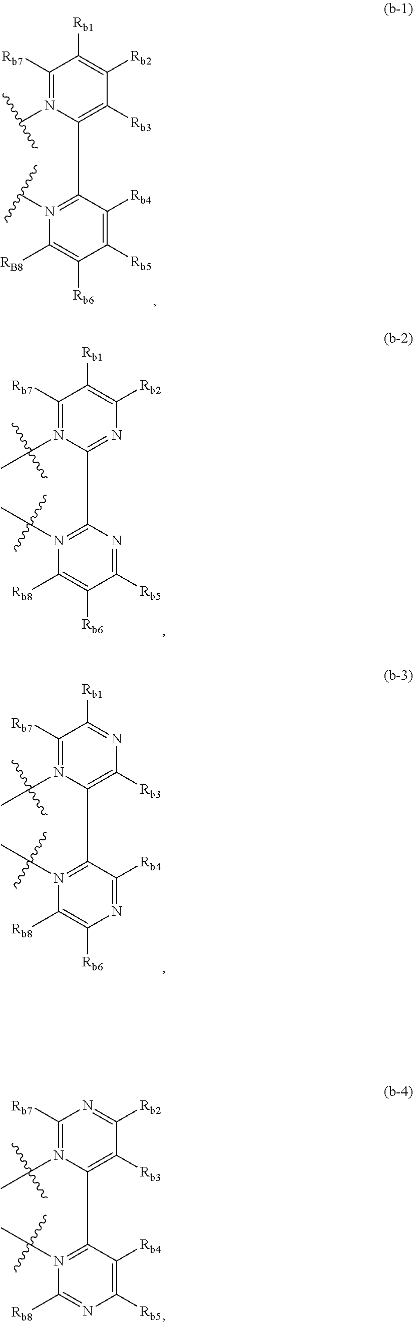

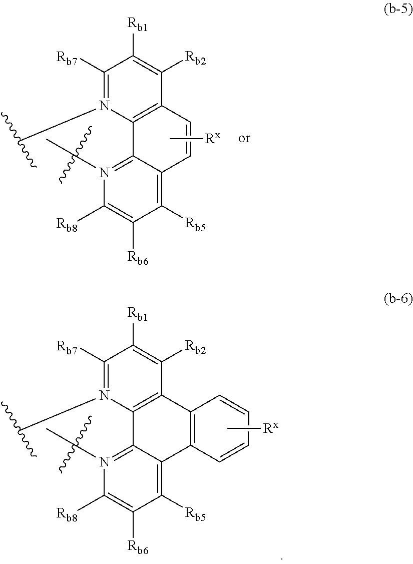

Preferably, LIG₂ of formula (b) corresponds to formula (b-1), (b-2), (b-3), (b-4) or (b-5), more preferably to formula (b-1) or (b-5), even more preferably to formula (b-1). According to the previous embodiments, when LIG₂ is of formula (b), R_{b1} to R_{b8} each independently represent advantageously H, halogen, optionally substituted C₁-C₆ alkyl, optionally substituted aryl, OR¹⁹ or NR²⁰R²¹, with R¹⁹ to R²¹ being preferably each independently selected in the group consisting of H and C₁-C₆ alkyl. Preferably, R_{b1} to R_{b8} each independently represent H, halogen, C₁-C₆ alkyl or aryl, more preferably H or aryl. R^{b1} to R_{b8} are typically identical. Even more preferably, R^{b1} to R_{b8} are H.

In the particular embodiment when LIG₂ is of formula (b-5) or (b-6), in particular of formula (b-5), R_{b1}, R_{b2}, R_{b5} to R_{b8} are as defined above and R^{x} represents advantageously one or several substituents selected in the group consisting of H, halogen, optionally substituted C₁-C₆ alkyl, optionally substituted aryl, OR¹⁹ and NR²⁰R²¹, with R¹⁹ to R²¹ being preferably each independently selected in the group consisting of H and C₁-C₆ alkyl. Preferably, R^{x} represents one or several substituents selected in the group consisting of H, C₁-C₆ alkyl and aryl, such as H or aryl, more preferably H. Even more preferably, when LIG₂ is of formula (b-5), LIG₂ represents:

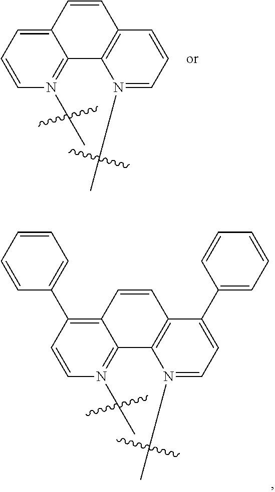

in particular

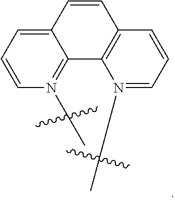

In a particularly preferred embodiment, LIG₂ is of formula (b-1) with R^{b1} to R_{b8} being H.

LIG₃

In a particular embodiment, LIG₃ is a bidentate ligand of the following formula (c):

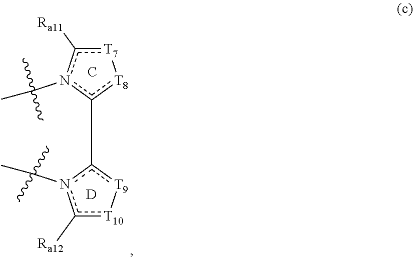

In particular, LIG3 of formula (c) may correspond to the following ligands:

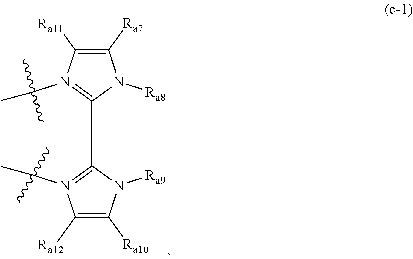

in particular

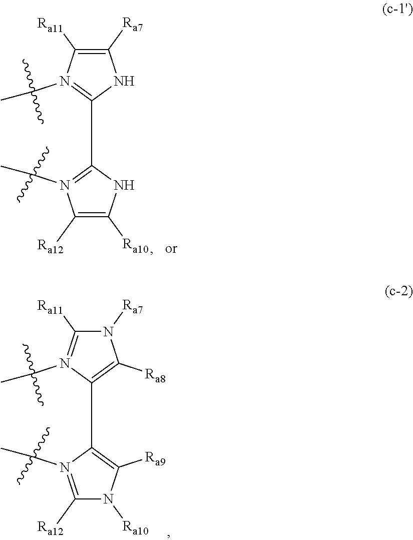

in particular

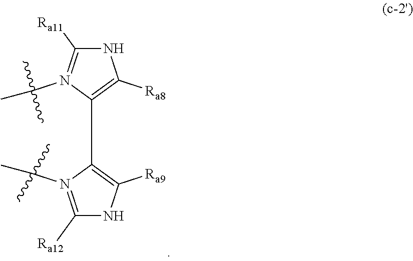

Advantageously, LIG₃ of formula (c) corresponds to formula (c-1), notably (c-1′). Preferably, when LIG₃ is of formula (c), in particular of formula (c-1), notably (c-1′), Rₐ₇ to Rₐ₁₂ each independently represent H, halogen, optionally substituted C₁-C₆ alkyl, optionally substituted aryl, OR¹⁹ or NR²⁰R²¹, with R¹⁹ to R²¹ being preferably each independently selected in the group consisting of H and C₁-C₆ alkyl. More preferably, Rₐ₇ to Rₐ₁₂ each independently represent H, halogen, C₁-C₆ alkyl, aryl, OR¹⁹ or NR²⁰R²¹, with R¹⁹ to R²¹ being as defined above. Even more preferably, Rₐ₇ to Rₐ₁₂ each independently represent H or C₁-C₆ alkyl. Typically, Rₐ₇ to Rₐ₁₂ are identical. In a preferred embodiment, Rₐ₇ to Rₐ₁₂ are H.

In a preferred embodiment, LIG₃ is a bidentate ligand of the following formula (d):

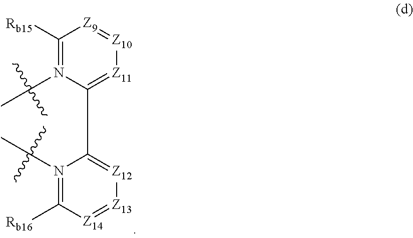

In particular, LIG₃ of formula (d) may correspond to the following ligands:

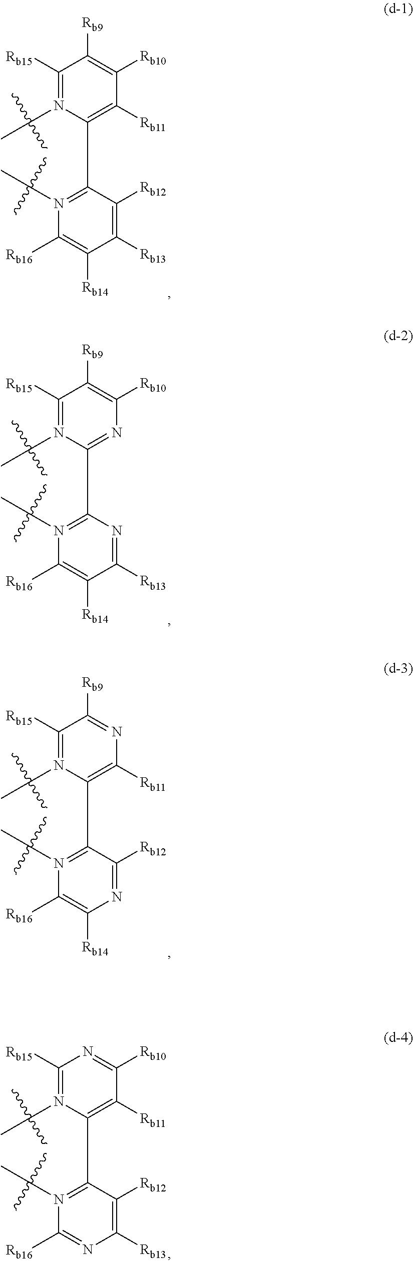

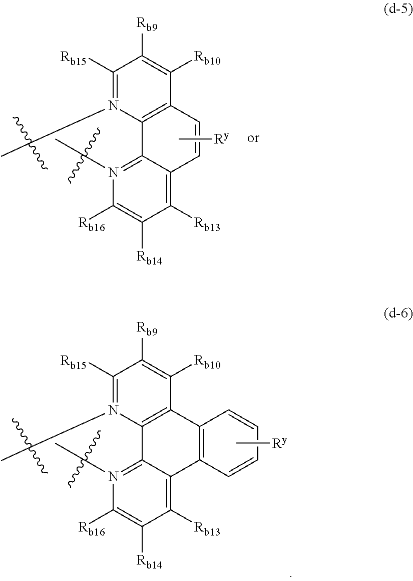

Preferably, LIG₃ of formula (d) corresponds to formula (d-1), (d-2), (d-3), (d-4) or (d-5), more preferably to formula (d-1) or (d-5), even more preferably to formula (d-1). According to the previous embodiments, when LIG₃ is of formula (d), R_{b9} to R_{b16} each independently represent advantageously H, halogen, optionally substituted C₁-C₆ alkyl, optionally substituted aryl, OR¹⁹ or NR²⁰R²¹, with R¹⁹ to R²¹ being preferably each independently selected in the group consisting of H and C₁-C₆ alkyl. Preferably, R_{b9} to R_{b16} each independently represent H, halogen C₁-C₆ alkyl or aryl, more preferably H or aryl. R_{b9} to R_{b16} are typically identical. Even more preferably, R_{b9} to R_{b16} are H.

In the particular embodiment when LIG₃ is of formula (d-5) or (d-6), in particular of formula (d-5), R_{b9}, R_{b10}, R_{b13} to R_{b16} are as defined above and R^{Y} represents advantageously one or several substituents selected in the group consisting of H, halogen, optionally substituted C₁-C₆ alkyl, optionally substituted aryl, OR¹⁹ and NR²⁰R²¹, with R¹⁹ to R²¹ being preferably each independently selected in the group consisting of H and C₁-C₆ alkyl. Preferably, R^{Y} represents one or several substituents selected in the group consisting of H, C₁-C₆ alkyl and aryl, more preferably H. Even more preferably, when LIG₃ is of formula (d-5), LIG₃ represents:

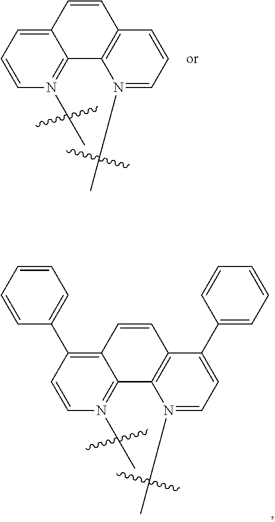

in particular

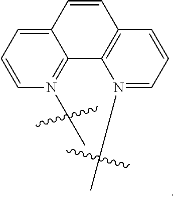

According to a preferred embodiment, compound of formula (I) corresponds to the following formula:

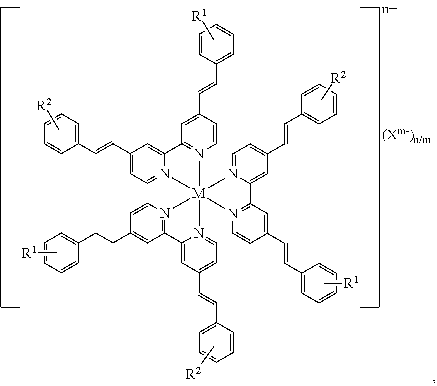

i.e. y1 is 3, y2 is 0 and y3 is 0, R¹ and R² are as defined above, typically R¹ and R² each independently represent OR⁹, R⁹ being preferably H or C₁₋C₆ alkyl, such as methyl, and M is as defined above, preferably ruthenium or osmium, typically ruthenium.

According to another preferred embodiment, compound of formula (I) corresponds to the following formula:

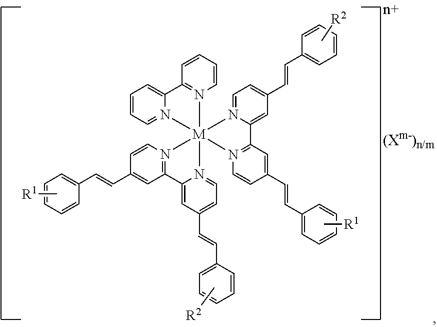

i.e. y1 is 2, y2 is 1 and y3 is 0, R¹ and R² are as defined above, typically R¹ and R² each independently represent OR⁹ or NR¹⁰R¹¹, in particular OR⁹, R⁹ to R¹¹ being preferably H or C₁-C₆ alkyl, such as methyl, and M is as defined above, preferably ruthenium or osmium, typically ruthenium.

According to another preferred embodiment, compound of formula (I) may be one of the following formulas:

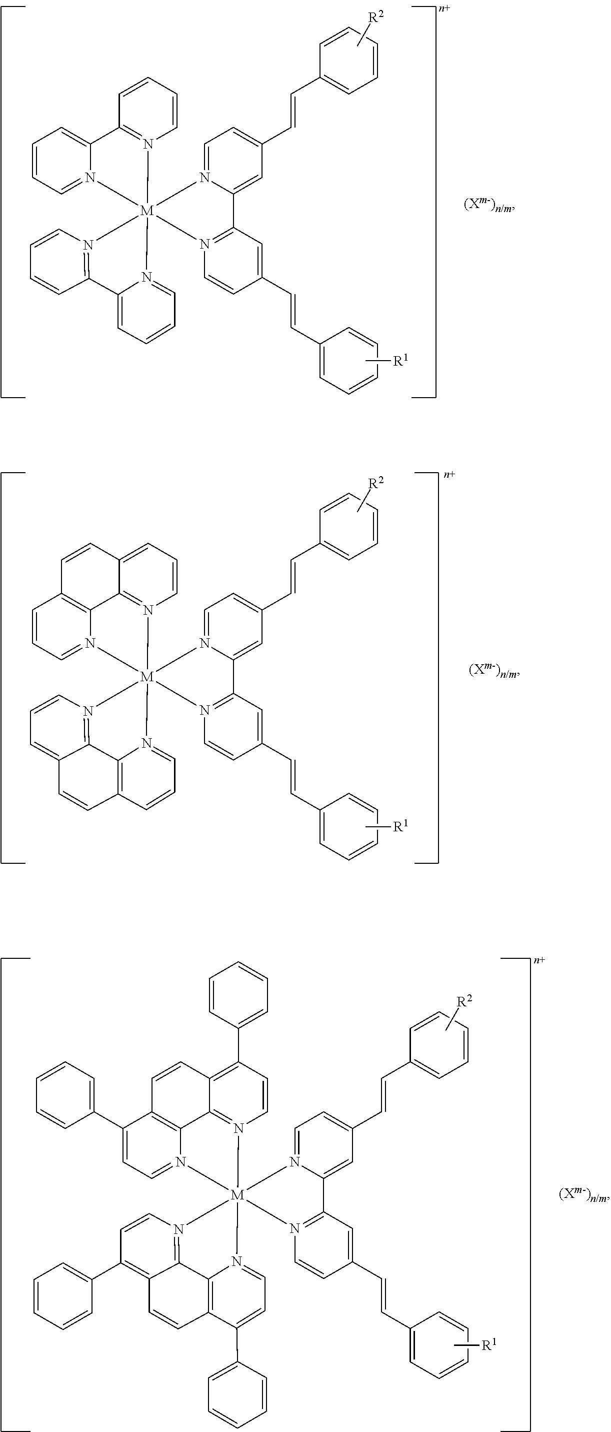

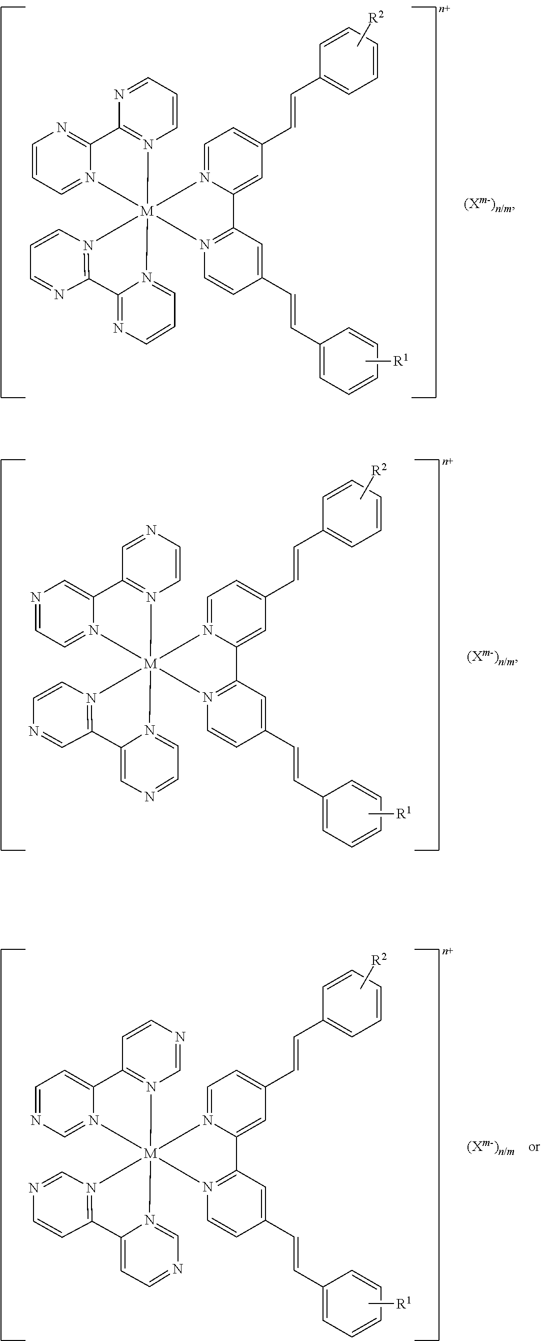

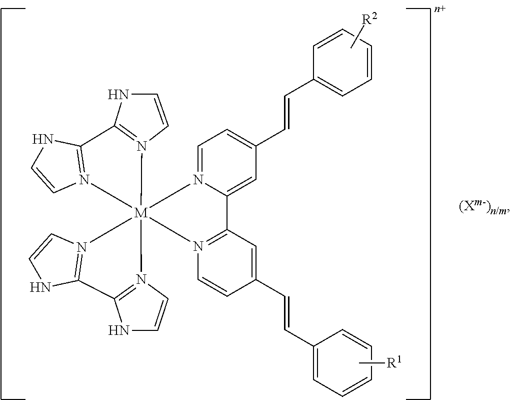

preferably

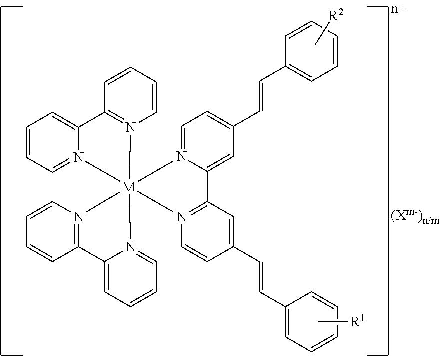

i.e. y1 is 1, y2 is 2, y3 is 0 R¹ and R² are as defined above, typically R¹ and R² each independently represent OR⁹ or NR¹⁰R¹¹, in particular OR⁹, R⁹ to R¹¹ being preferably H or C₁-C₆ alkyl, such as methyl, and M is as defined above, preferably ruthenium or osmium, typically ruthenium.

According to a particular embodiment, the present invention relates to the following compounds of formula (I):

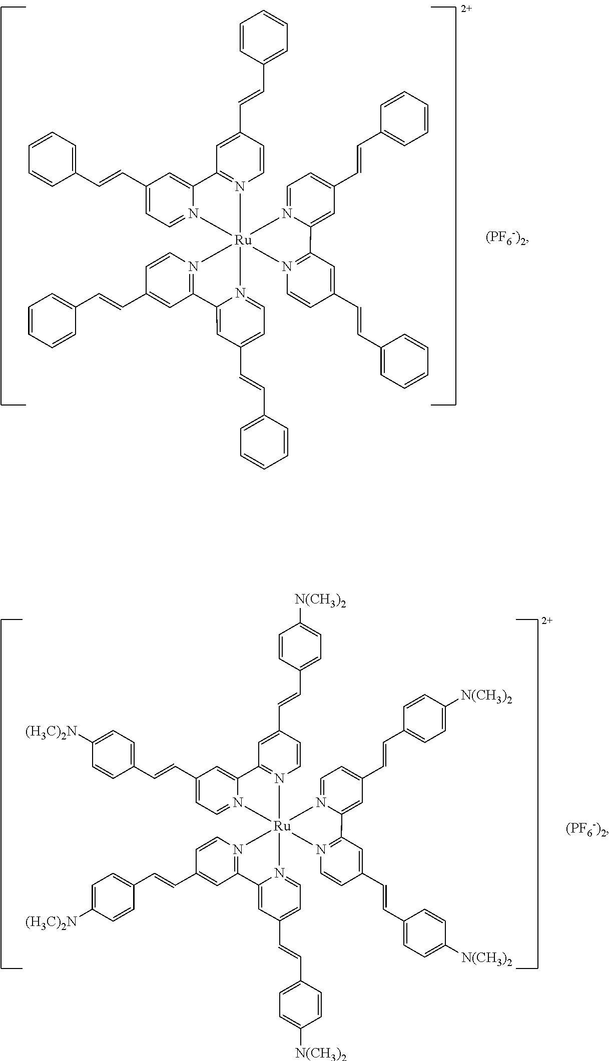

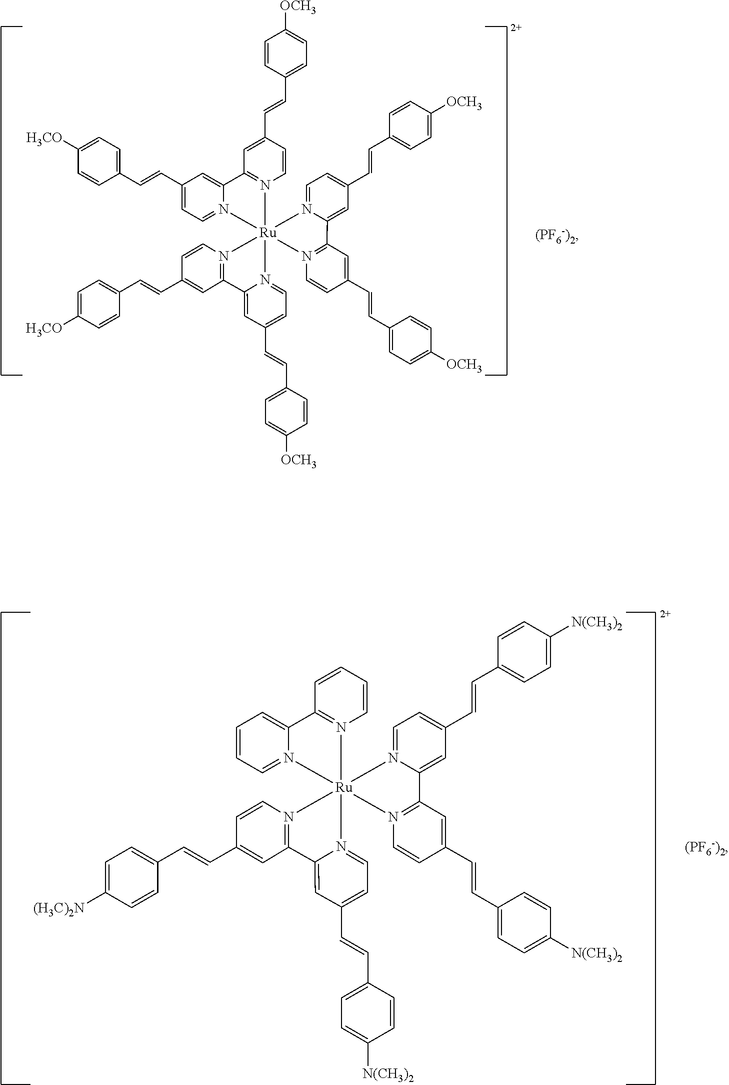

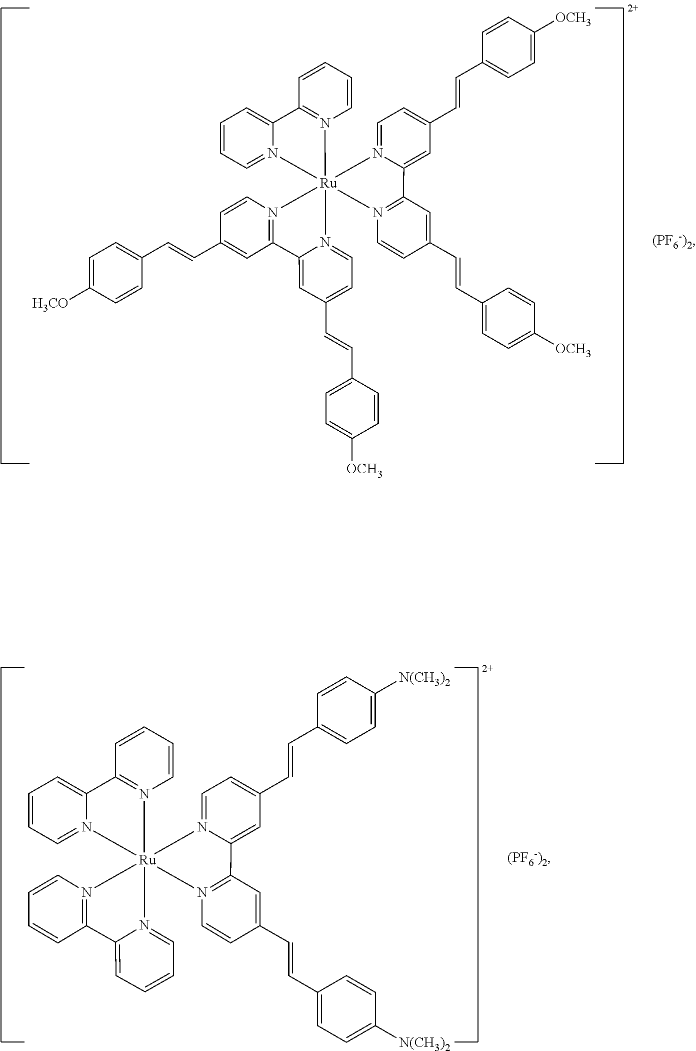

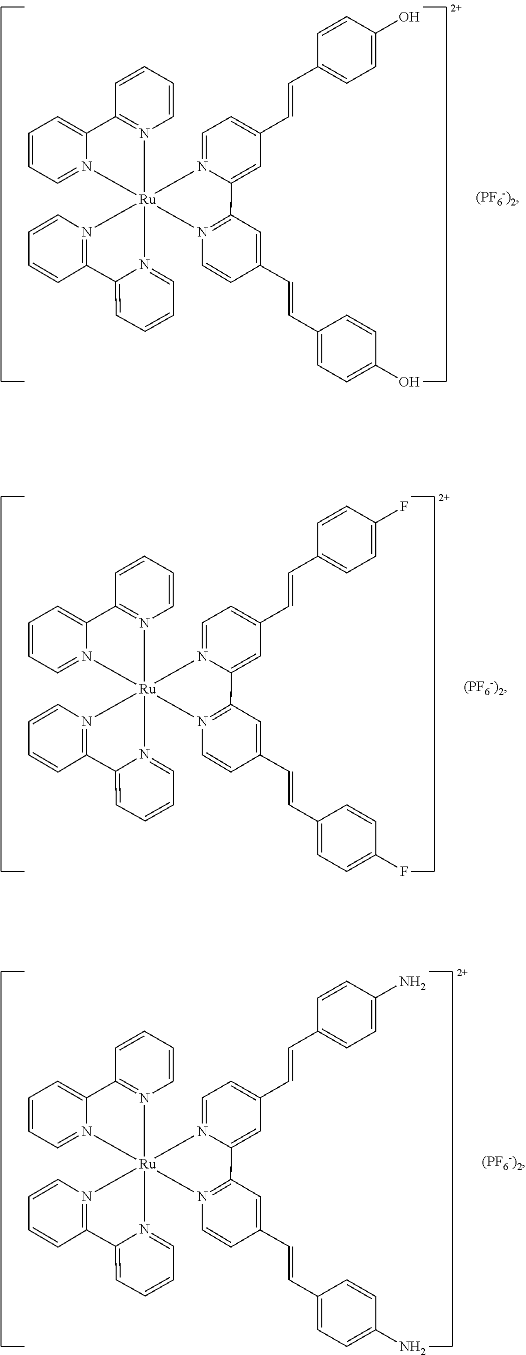

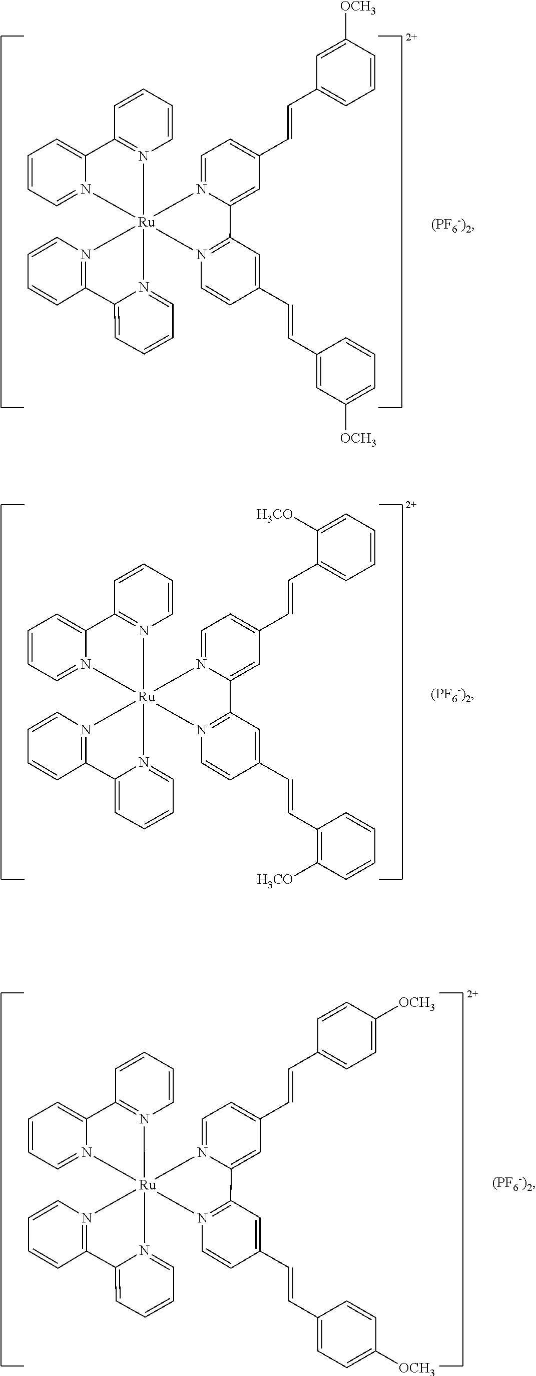

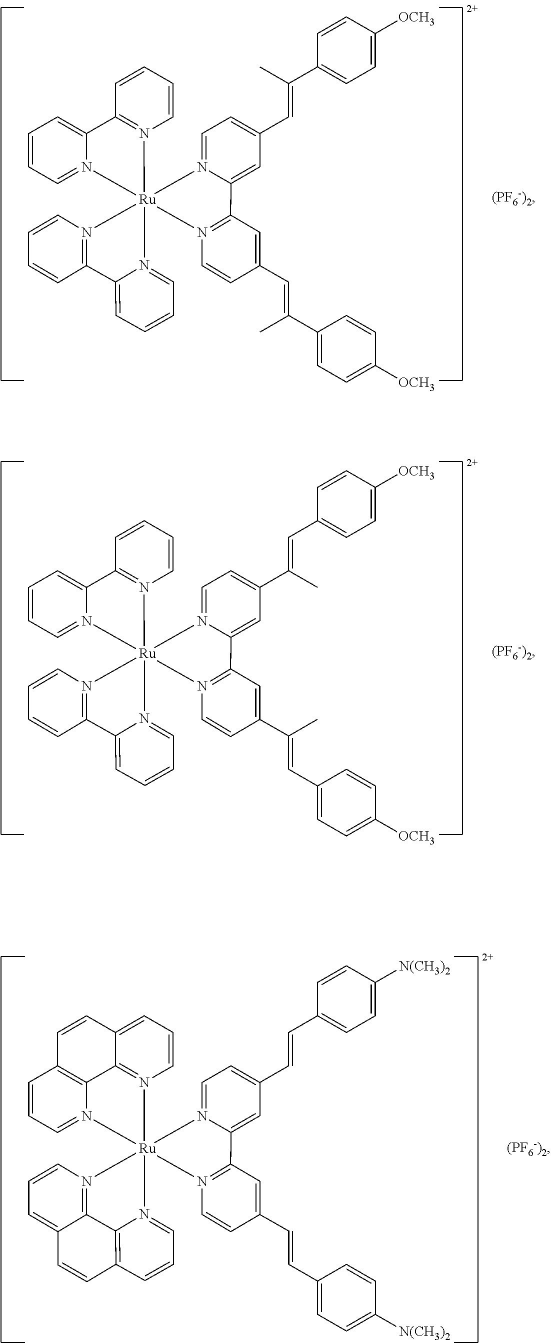

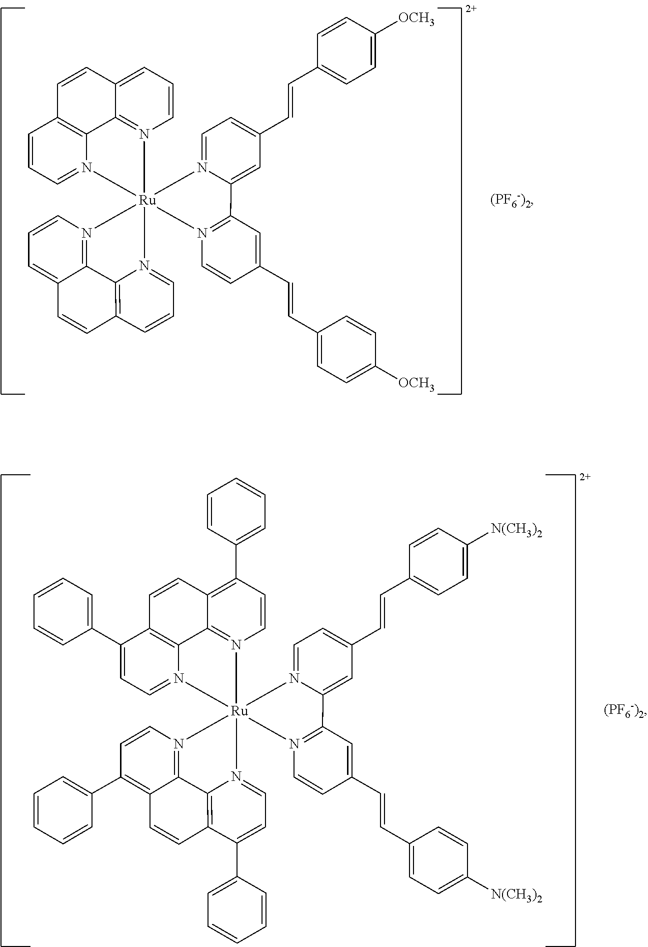

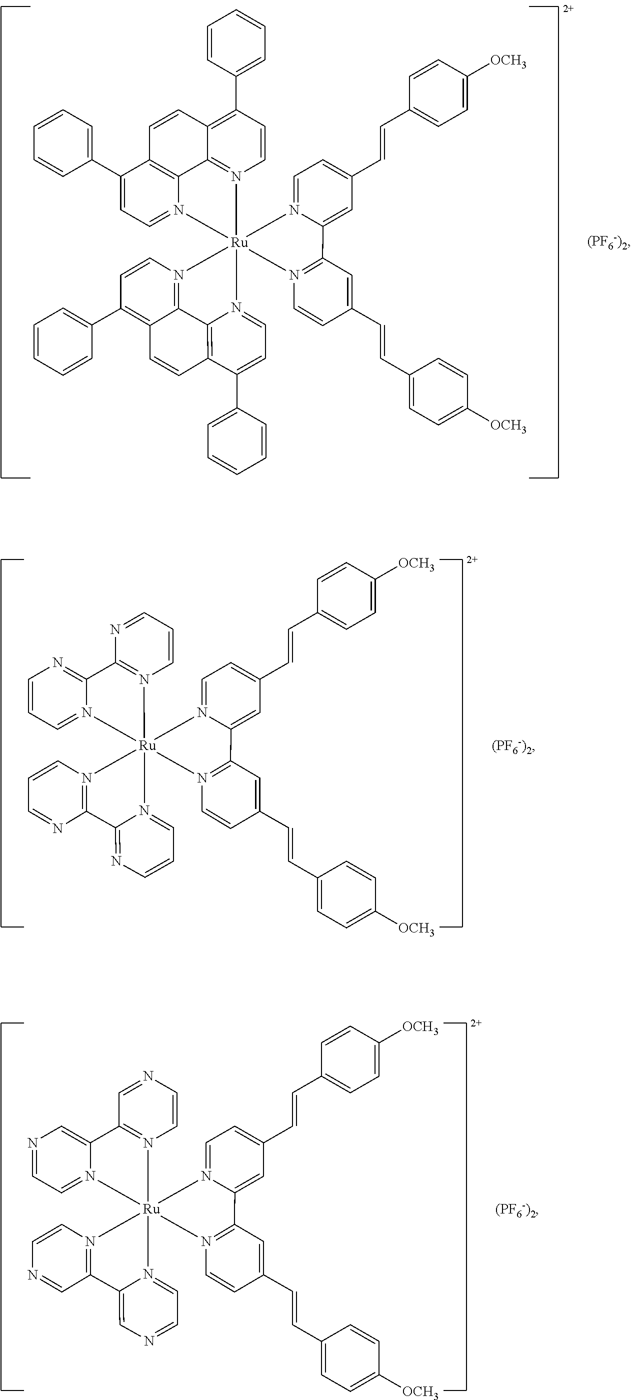

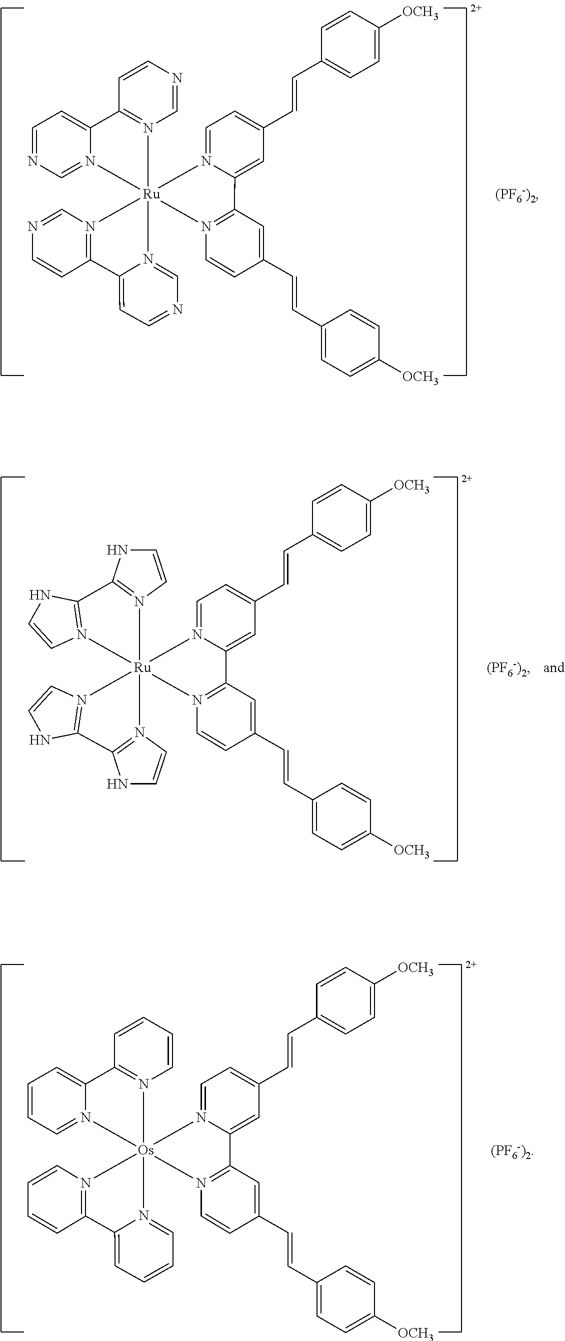

Pharmaceutical Composition

The present invention also relates to a pharmaceutical composition comprising at least one pharmaceutically acceptable excipient and at least one compound of formula (I) as described above or a pharmaceutically acceptable salt and/or solvate thereof.

The pharmaceutical compositions of the invention can be intended to oral or parenteral (e.g. subcutaneous, intramuscular, intravenous) administration, preferably oral or intravenous administration. The active ingredient can be administered in unit forms for administration, mixed with conventional pharmaceutical carriers, to animals, preferably mammals including humans.

For oral administration, the pharmaceutical composition can be in a solid or liquid (solution or suspension) form.

A solid composition can be in the form of tablets, gelatin capsules, powders, granules and the like. In tablets, the active ingredient can be mixed with pharmaceutical vehicle(s) such as gelatin, starch, lactose, magnesium stearate, talc, gum arabic and the like before being compressed. The tablets may be further coated, notably with sucrose or with other suitable materials, or they may be treated in such a way that they have a prolonged or delayed activity. In powders or granules, the active ingredient can be mixed or granulated with dispersing agents, wetting agents or suspending agents and with flavor correctors or sweeteners. In gelatin capsules, the active ingredient can be introduced into soft or hard gelatin capsules in the form of a powder or granules such as mentioned previously or in the form of a liquid composition such as mentioned below.

A liquid composition can contain the active ingredient together with a sweetener, a taste enhancer or a suitable coloring agent in a solvent such as water. The liquid composition can also be obtained by suspending or dissolving a powder or granules, as mentioned above, in a liquid such as water, juice, milk, etc. It can be for example a syrup or an elixir.

For parenteral administration, the composition can be in the form of an aqueous suspension or solution which may contain suspending agents and/or wetting agents. The composition is advantageously sterile. It can be in the form of an isotonic solution (in particular in comparison to blood).

The compounds of the invention can be used in a pharmaceutical composition at a dose ranging from 0.01 mg to 1000 mg a day, administered in only one dose once a day or in several doses along the day, for example twice a day in equal doses. The daily administered dose is advantageously comprised between 5 mg and 500 mg, and more advantageously between 10 mg and 200 mg. However, it can be necessary to use doses out of these ranges, which could be noticed by the person skilled in the art.

According to a particular embodiment, the compound of formula (I) or a pharmaceutically acceptable salt and/or solvate thereof, or the pharmaceutical composition according to the present invention is encapsulated within a polymeric matrix, such as 1,2-distearoyl-sn-glycero-3-phosphoethanolamine-N-[biotin(polyethylene glycol)-2000] ammonium salt (DSPE-PEG₂₀₀₀-biotin). Such an encapsulation can be performed according to methods well-known from the skilled person, in particular according to methods described in Gasser, G. et al., Angew. Chem. Int. Ed, 2019 and in Gasser, G. et al. Inorg Chem. 2019. The encapsulation is particularly useful for controlled, targeted and/or extended release of compound of formula (I) in the body of the subject in need thereof.

Treatment

The compound of formula (I) or a pharmaceutically acceptable salt and/or solvate thereof, or the pharmaceutical composition according to the present invention is useful as a drug.

According to a preferred embodiment, the compound of formula (I), or a pharmaceutically acceptable salt and/or solvate thereof, or the pharmaceutical composition according to the present invention is useful as a photosensitizer agent in photodynamic therapy. It is particularly intended to treat by photodynamic therapy a disease selected from cancer, such as lung cancer, bladder cancer, oesophageal cancer, colon cancer, stomach cancer, liver cancer, skin cancer, ovarian cancer, pancreatic cancer, head and neck cancer, or brain cancer; bacterial infection, such as sinusitis, diabetic feet, burned wounds; fungal infection, such as mycoses; viral infection such as herpes; and skin disorders, such as acne, port wine stains.

Methods of Preparation of a Compound of Formula (I-A), (I-B), (I-C) or (I-D)

The present invention relates also to a first method of preparation of a compound of the following formula (I-A) or (I-D):

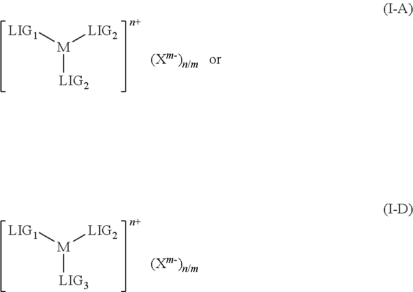

or a pharmaceutically acceptable salt and/or solvate thereof, and thus more particularly to a compound of formula (I) or a pharmaceutically acceptable salt and/or solvate thereof, where y1=1 and thus y2+y3=2, wherein LIG₁ and LIG₂ are as defined above and M is ruthenium or osmium, preferably ruthenium, said method comprising the following steps:
(a) reacting a compound of the following formula (II)

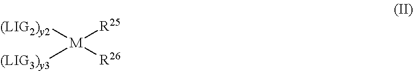

in which LIG₂, LIG₃, y2 and y3 are as defined above,

R²⁵ and R²⁶ each independently represent halogen, OR²⁷ or S(O)R²⁸R²⁹,

R²⁷ is H or C₁-C₆ alkyl, R²⁸ and R²⁹ are each independently a (C₁-C₆)alkyl, preferably a methyl,

with a compound of formula (III)

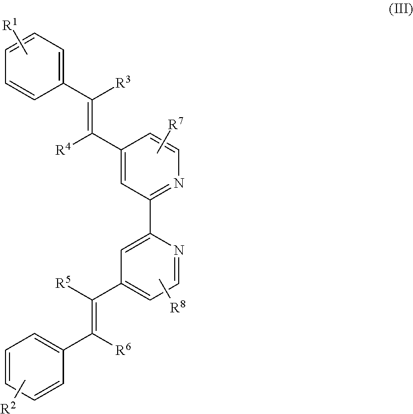

in which R¹ to R⁸ are as defined above, then
(b) reacting the product resulting from step (a) with a salt A^{m+}X^{m-}, wherein X^{m-} is as defined above and A^{m+} is a counter cation.

Step (a)

In the compound of formula (II), R²⁵ and R²⁶ are preferably identical and/or both represent a halogen, such as Cl. Compound of formula (II) can be obtained using suitable ligands according to methods described in the literature, in particular in Meyer, T. et al., 1978 and in McCusker, C. E and McCusker, J. K, 2011. For example, when R²⁵=R²⁶=halogen such as Cl and preferably M=Ru, compound of formula (II) can be prepared by reacting LIG₂ and/or LIG₃ with M(R²⁵)₃ such as Ru(Ill)Cl₃, notably in the presence of LiCl. Compound of formula (II) with R²⁵=R²⁶=halogen such as Cl and preferably M=Ru can also be prepared by reacting LIG₂ and/or LIG₃ with M(R^{25′})₄(R²⁵)₂ (with R^{25′} being OR²⁷ or S(O)R²⁸R²⁹) such as Ru(II)Cl₂DMSO₄.

Compound of formula (II) advantageously corresponds to the following compounds (II-A), (II-B) or (II-C):

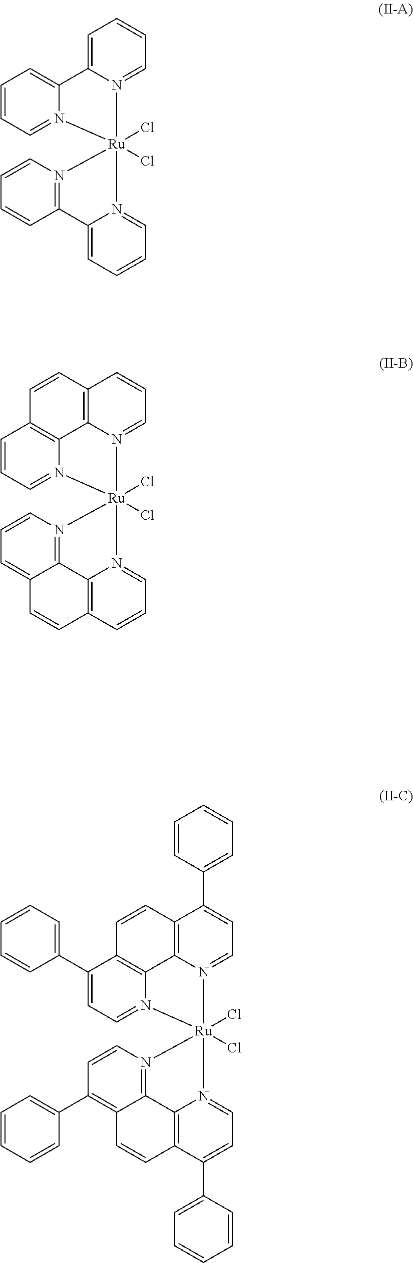

Compound of formula (II-A), also called Ru(bipy)₂Cl₂, compound of formula (II-B), also called Ru(phen)₂Cl₂ or dichlorobis(1,10-phenantroline)ruthenium(II), and compound of formula (II-C), also called Ru(4,7-diphenyl-1,10-phenantroline)₂Cl₂ or dichlorobis(4,7-diphenyl-1,10-phenantroline)ruthenium(II), are synthesized as previously published using the respective ligands (McCusker, C. E. and McCusker, J. K., 2011). Compound of formula (II) preferably corresponds to formula (II-A).

Compound of formula (III) can be obtained using suitable substituents R¹ to R⁸ according to methods described in the literature (Meyer, T. J. et al., 1987, Wu, Q. et al., 1995). According to a preferred embodiment, compound of formula (III) corresponds to the following compound (III-A):

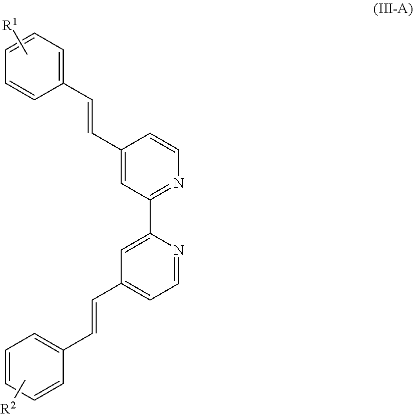

in which R¹ and R² are as defined above. Preferably R¹ and R² are identical.

The inventors have also developed a novel synthesis of a compound of formula (III-A) wherein R¹ and R² are identical to avoid tedious purifications, multistep synthesis and overall low yields of the prior art (Meyer, T. J. et al., 1987, Wu, Q. et al., 1995). Thus, the present invention relates also to a method of preparation of a compound of formula (III-A) wherein R¹ and R² are identical comprising the step of reacting one equivalent of 4,4′-dimethyl-2,2′-bipyridine (no CAS 1134-35-6) in the presence of a strong base with a compound of following formula (IV):

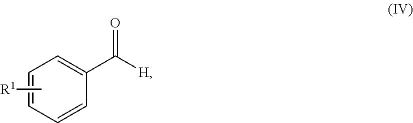

in which R¹, identical to R², is as defined above.

Such a method has the advantage to be carried out in one step and in mild conditions. Advantageously, the strong base is non nucleophilic. The strong base is preferably selected in the group consisting of lithium diisopropylamide (LDA), sodium bis(trimethyl)silylamide (NaHMDS), potassium bis(trimethyl)silylamide (KHMDs), sodium hydride (NaH), potassium hydride (KH) and potassium tert-butoxide (^{t}BuOK). More preferably, the strong base is potassium tert-butoxide.

Advantageously, the reaction is carried out with 2 to 3 equivalents, preferably 2 to 2.5 of a compound of formula (IV) compared to 4,4′-dimethyl-2,2′-bipyridine.

The reaction is typically carried out with 3 to 5 equivalents of the strong base, preferably with 4 equivalents, compared to 4,4′-dimethyl-2,2′-bipyridine.

The reaction is preferably carried out in a polar solvent, in particular an aprotic polar solvent such as dimethylformamide (DMF), dimethylsulfoxide (DMSO), pyridine, acetonitrile, ethyl acetate, acetone, butanone and mixtures thereof. Preferably, the solvent is DMF.

The reaction is preferably carried out under inert atmosphere such as nitrogen (N₂) or argon (Ar) atmosphere.

Step (a) corresponds to a ligand exchange wherein substituents R²⁵ and R²⁶ are replaced by the bidentate ligand LIG₁ as described above in compound of formula (I-A). Eventually, additional steps of protection/deprotection and/or of functionalization well-known from the skilled person in the art may occur between steps (a) and (b) to afford compound of formula (I-A) with the suitable substituents as described above. The reaction is preferably carried out with 1 to 1.5, more preferably 1 to 1.2 equivalent of compound of formula (III) compared to compound of formula (II).

The reaction is preferably carried out in a polar solvent, preferably selected among water, alcohols, such as methanol, ethanol, propanol, butanol, and mixtures thereof. Preferably, the solvent is an alcohol, in particular ethanol.

The reaction is preferably carried out under inert atmosphere such as nitrogen (N₂) or argon (Ar) atmosphere.

The reaction is preferably carried out at a temperature corresponding to the boiling temperature of the solvent.

Step (b)

X^{m-} is a pharmaceutically acceptable anion, preferably selected in the group consisting of PF₆⁻, Cl⁻, Br⁻, I⁻, BF₄⁻, (C₁-C₆ alkyl)-C(O)O⁻, (C₁-C₆ haloalkyl)-C(O)O⁻, (C₁-C₆-alkyl)-SO₃, (C₁-C₆-haloalkyl)-SO₃⁻, SO₄²⁻ and PO₄³⁻, in particular PF₆⁻, Cl⁻, Br⁻, BF₄⁻, CH₃C(O)O⁻, CF₃C(O)O— and CF₃SO₃, more preferably X^{m-} is PF₆⁻. A^{m+} is a counter cation preferably selected among (N⁺R′R″R′″R″″)ₘ, (H⁺)ₘ, (Na⁺)ₘ, (K⁺)ₘ and (Li⁺)ₘ, wherein m is 1, 2 or 3 and R′, R″, R′″ and R″″ are H or C₁-C₆ alkyl. Preferably (N+R′R″R′″R″″)ₘ corresponds to (NH₄⁺)ₘ or (NBu₄⁺)ₘ.

The salt A^{m+}X^{m-} is thus preferably selected among the salts, but not limited to, NH₄PF₆, NBu₄PF₆, KCl, KBr, LiCl, LiBr, HBF₄, NaOC(O)CH₃, KOC(O)CH₃, NH₄OCOCH₃, Na₂SO₄, H₃PO₄. Preferably, the salt used in step (b) is NH₄PF₆.

The present invention also relates to a second method of preparation of a compound of the following formula (I-B):

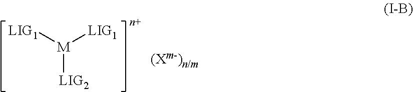

or a pharmaceutically acceptable salt and/or solvate thereof, wherein LIG₁ and LIG₂ are as defined above and M is ruthenium or osmium, preferably ruthenium, said method comprising the following steps:
(c) reacting of a compound of formula (III)

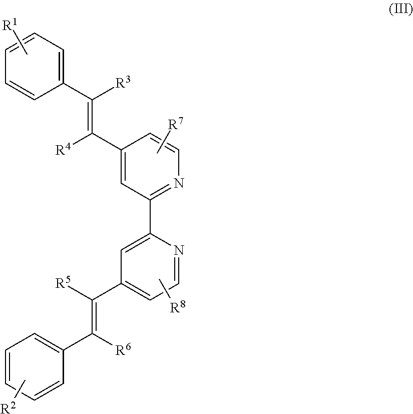

in which R¹ to R⁸ are as defined above,

with of a compound of the following formula (V):

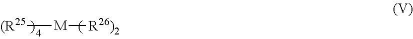

in which R²⁵ and R²⁶ are as defined above provided that R²⁵ and R²⁶ are not identical, then
(d) reacting the compound resulting from step (c) with a compound of formula (VI) or (VII):

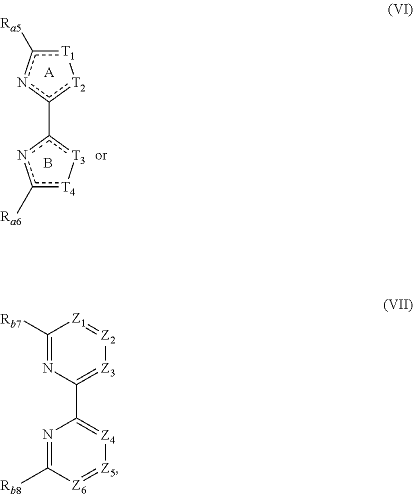

in which [symbol] , T₁ to T₄, Z₁ to Z₆, Rₐ₅, Rₐ₆, R_{b7} and R_{b8} are as defined above, then
(e) reacting the product resulting from step (d) with a salt A^{m+}X^{m-}, wherein X^{m-} is as defined above and A^{m+} is a counter cation.

Step (c)

Compound of formula (III) can be obtained as mentioned previously.

In the compound of formula (V), R²⁵ and R²⁶ are preferably selected in the group consisting of halogen, such as Cl, and S(O)R²⁸R²⁹, such as S(O)(CH₃)₂. Advantageously, R²⁵ is a better leaving group than R²⁶, so that R²⁵ can represent for example S(O)R²⁸R²⁹, such as S(O)(CH₃)₂, and R²⁶ can represent halogen, such as Cl. Compound of formula (VI) advantageously corresponds to Ru(DMSO)₄Cl₂ (no CAS: 67-68-5). Ru(DMSO)₄Cl₂ can also be synthesized as previously published (Bratsos, I. and Alessio, E., 2010).

Step (c) corresponds to a ligand exchange wherein the four substituents R²⁵ are replaced by two bidentate ligands LIG₁ as described above in compound of formula (I-B).

Eventually, additional steps of protection/deprotection and/or of functionalization well-known from the skilled person in the art may occur between steps (c) and (d) to afford at the end of step (e) compound of formula (I-B) with the suitable substituents as described above.

The reaction is preferably carried out with compound of formula (III) in excess, preferably with 1.5 to 3 equivalents, more preferably with 2 equivalents, compared to compound of formula (V).

The reaction is preferably carried out in a polar solvent, in particular an aprotic polar solvent such as dimethylformamide (DMF), dimethylsulfoxide (DMSO), pyridine, acetonitrile, ethyl acetate, acetone, butanone and mixtures thereof. Preferably, the solvent is DMF.

The reaction is preferably carried out under inert atmosphere such as nitrogen (N₂) or argon (Ar) atmosphere.

The reaction is preferably carried out at a temperature corresponding to the boiling temperature of the solvent.

Step (d)

The intermediate resulting from step (c) is typically of the following formula:

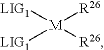

in which LIG₁ and R²⁶ are as described above.

Preferably, LIG₁ is of formula (LIG1-A):

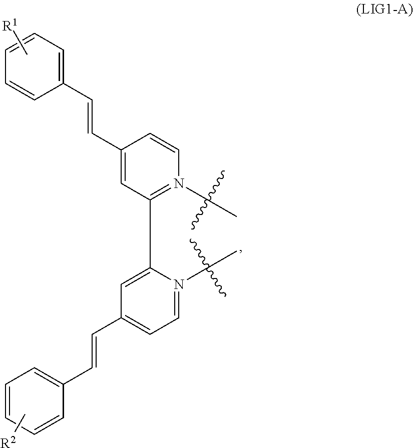

with R¹ and R² being as described above, advantageously being identical.

In this intermediate, R²⁶ preferably represents a halogen, such as Cl.

Step (d) corresponds to a ligand exchange wherein the two substituents R²⁶ are replaced by a bidentate ligands LIG₂ as described above.

Preferably, the compound resulting from step (c) reacts with 1 to 1.5 equivalent of compound of formula (VI) or (VII), more preferably with 1.2 equivalent compared to compound of formula (V).

Eventually, additional steps of protection/deprotection and/or of functionalization well-known from the skilled person in the art may occur between steps (d) and (e) to afford at the end of step (e) compound of formula (I-B) with the suitable substituents as described above.

The reaction is preferably carried out in a polar solvent, preferably selected among water, alcohols, such as methanol, ethanol, propanol, butanol, and mixtures thereof. Preferably, the solvent is an alcohol, in particular ethanol.

The reaction is preferably carried out under inert atmosphere such as nitrogen (N₂) or argon (Ar) atmosphere.

The reaction is preferably carried out at a temperature corresponding to the boiling temperature of the solvent.

Step (e)

Step (e) involves the same embodiments as those described for step (b) above.

The present invention also relates to a third method of preparation of a compound of the following formula (I-B):

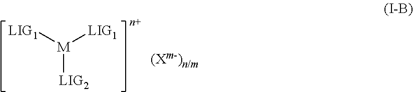

or a pharmaceutically acceptable salt and/or solvate thereof, wherein LIG₁ and LIG₂ are as defined above and M is ruthenium or osmium, preferably ruthenium, said method corresponding to the first method described above for preparing a compound of formula (I-A), in which LIG₁ and LIG₂ are switched.

The present invention relates to a fourth method of preparation of a compound of the following formula (I-C):

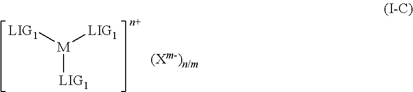

or a pharmaceutically acceptable salt and/or solvate thereof, wherein LIG₁ is as defined above and M is ruthenium or osmium, preferably ruthenium, said method comprising the following steps:
(f) reacting one equivalent of a compound of formula (V)

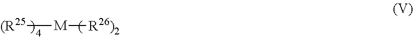

in which R²⁵ and R²⁶ are as defined above provided that R²⁵ and R²⁶ are not identical, with a compound of formula (III)

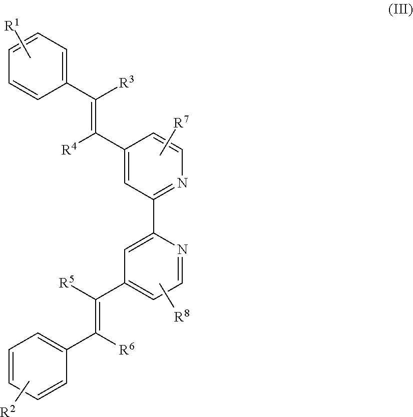

in which R¹ to R⁸ are as defined above, then
(g) reacting the product resulting from step (a) with a salt A^{m+}X^{m-}, wherein X⁻ is as defined above and A^{m+} is a counter cation.

Step (f)

Compound of formula (III) can be obtained as mentioned above.

In the compound of formula (V), R²⁵ and R²⁶ are preferably selected in the group consisting of halogen, such as Cl, and S(O)R²⁸R²⁹, such as S(O)(CH₃)₂. Advantageously, R²⁵ is a better leaving group than R²⁶, so that R²⁵ can represent for example S(O)R²⁸R²⁹, such as S(O)(CH₃)₂, and R²⁶ can represent halogen, such as Cl. Compound of formula (VI) advantageously corresponds to Ru(DMSO)₄Cl₂ (no CAS: 67-68-5).

Step (f) corresponds to a ligand exchange wherein the six substituents R²⁵ and R²⁶ are replaced by three bidentate ligands LIG₁ as described above in compound of formula (I-C).

Eventually, additional steps of protection/deprotection and/or of functionalization well-known from the skilled person in the art may occur between steps (f) and (g) to afford at the end of step (e) compound of formula (I-C) with the suitable substituents as described above.

The reaction is preferably carried out with 3 to 6 equivalents, more preferably with four equivalents of compound of formula (III), compared to compound of formula (V).

The reaction is preferably carried out in a protic polar solvent, preferably selected among water, alcohols, such as methanol, ethanol, propanol, butanol, and mixtures thereof. In another embodiment, the reaction can be carried out in an aprotic polar solvent such as dimethylformamide (DMF), dimethylsulfoxide (DMSO), pyridine, acetonitrile, ethyl acetate, acetone, butanone and mixtures thereof. Preferably, the solvent is an alcohol, in particular ethanol, or DMF.

The reaction is preferably carried out under inert atmosphere such as nitrogen (N₂) or argon (Ar) atmosphere.

The reaction is preferably carried out at a temperature corresponding to the boiling temperature of the solvent.

The above described methods of preparation of compounds of formula (I), i.e. compounds of formulas (I-A), (I-B), (I-C) and (I-D), can be applied for others metals than ruthenium or osmium selected from rhenium, rhodium, iridium and platinum. The skilled person knows the suitable reagents to be used, notably in place of reagents of formula (II) and (V).

According to the previous embodiments, in methods of preparation of a compound of formula (I), i.e. compounds of formulas (I-A), (I-B), (I-C) and (I-D), a compound obtained at the end of a reacting step can be separated from the reaction medium by methods well known to the person skilled in the art, such as by extraction, evaporation of the solvent or by precipitation or crystallisation (followed by filtration).

Said compound can be also purified if necessary by methods well known to the person skilled in the art, such as by recrystallisation, by distillation, by chromatography on a column of silica gel or by high performance liquid chromatography (HPLC).

### DESCRIPTION OF THE FIGURES

FIG. 1: Absorption spectra in CH₃CN of compounds a) 1-4, b) 5-8, c) 9-11.

FIG. 2: One- (OPM, λₑₓ=458 nm, λₑₘ=600-750 nm) and two-photon (TPM, λₑₓ=800 nm, λₑₘ=600-750 nm) excited Z-stack images in HeLa MCTS after incubation of compound 7 after 12 h (20 μM, 2% DMSO, v %). a) Z-axis images scanning from the top to the bottom of an intact spheroid. b) 3D z-stack of an intact spheroid.

FIGS. 3-5: Tumour growth inhibition assay. Change of the volume in HeLa MCTS in correlation to the time of the treatment. The MCTS were treated with compounds 1-3 (20 μM, 2% DMSO, v %) for FIG. 3, compounds 4-7 (20 μM, 2% DMSO, v %) for FIG. 4 and H₂TPP (20 μM, 2% DMSO, v %) or cisplatin (10 μM Pt-10 and 30 μM Pt-30) for FIG. 5. The MCTS were a) strictly kept in the dark, b) exposed to 1P irradiation (500 nm, 10 J/cm²), c) exposed to 2P irradiation (800 nm, 10 J/cm² with a section interval of 5 μm) on day 3. The error bars correspond to the standard deviation of the three replicates.

FIG. 6: Representative image of a viability assay in HeLa MCTS. MCTS were treated with compounds 1-7 (20 μM, 2% DMSO, v %) in the dark for three days. After this time, MCTS were kept in the dark, exposed to 1P irradiation (500 nm, 10 J/cm²) or to 2P irradiation (800 nm, 10 J/cm², section interval of 5 μm). After two days, the cell viability was assessed by measurement of the fluorescence of calcein (λₑₓ=495 nm, λₑₘ=515 nm), which is generated in living cells from calcein AM.

FIG. 7: PDT in vivo. The mice were randomly allocated into six different treatments: (i) 7-injected with 2P irradiation (7+TP), (ii) 7-injected with 1P irradiation (7+OP), (iii) physiological saline and 2P irradiation (TP), (iv) physiological saline and 1P light irradiation (TP), (v) 7-injected only (7) and (vi) physiological saline injected only (control). (A) In vivo tumour growth inhibition and (B) body weight curves for different treated mice. (C) Representative photographs of SW620/AD300 tumors in mice with different treatments.

FIG. 8: Absorption spectra in CH₃CN of compounds a) 12-14, b) 15-17, c) 18-20.

The present invention is illustrated by the following examples.

### EXAMPLES

1. Synthesis

The synthesis of the complexes is presented.

Structures

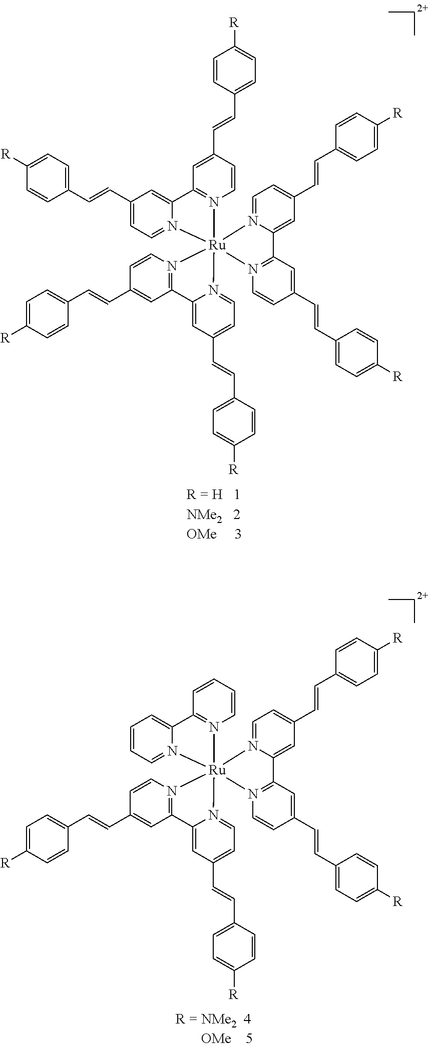

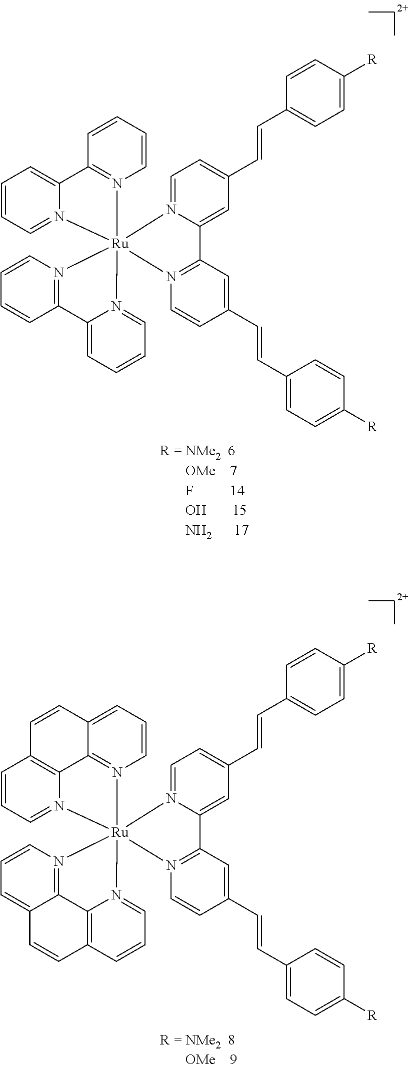

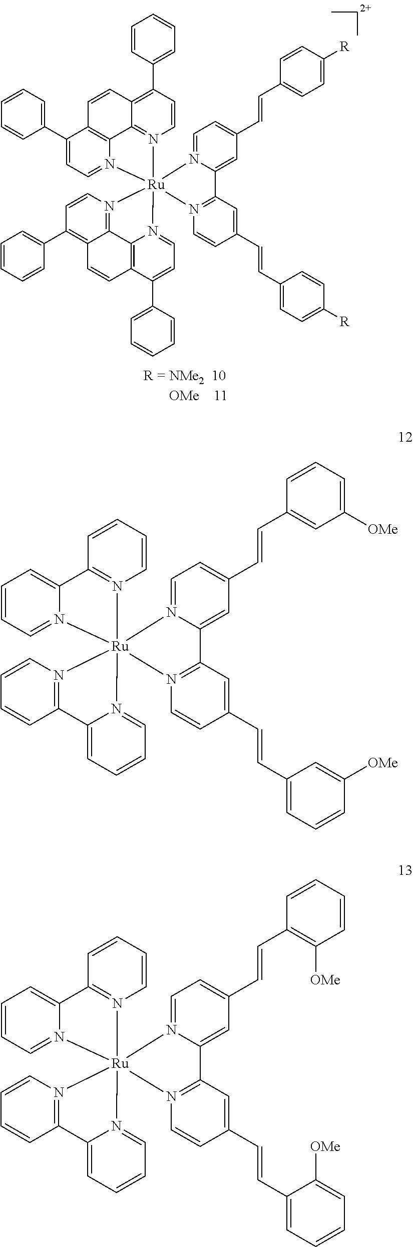

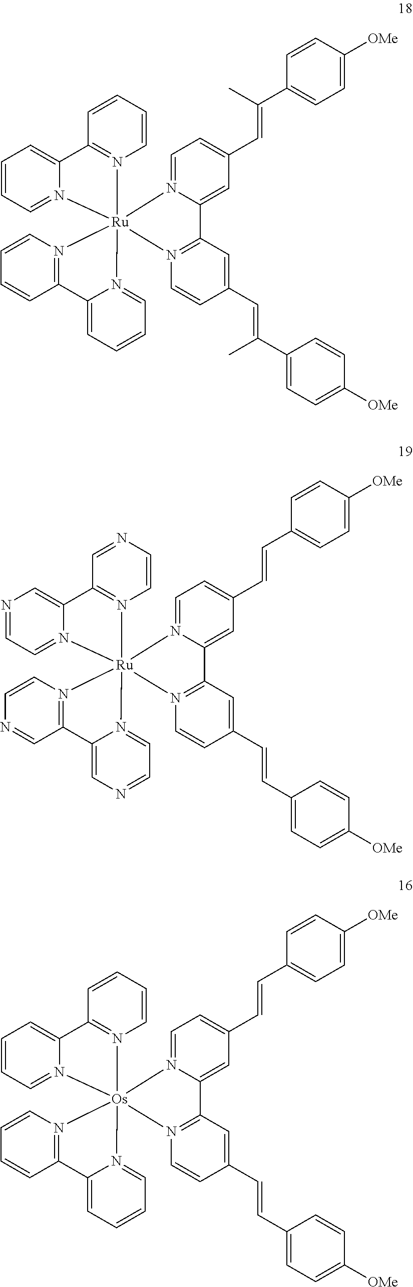

Materials

All chemicals were obtained from commercial sources and used without further purification. Solvents were dried over molecular sieves if necessary. The Ru(II) precursors Ru(DMSO)₄Cl₂ and Ru(bipy)₂Cl₂, Ru(phen)₂Cl₂ and Ru(bphen)₂Cl₂ were synthesised as previously reported using the respective ligand (Bratsos, I. and Alessio, E., 2010, Meyer, T. J. et al., 1987 and Wu, Q. et al, 1995).

Instrumentation and Methods

¹H and ¹³C NMR spectra were recorded on a Bruker 400 MHz or 500 MHz NMR spectrometer. Chemical shifts (5) are reported in parts per million (ppm) referenced to tetramethylsilane (δ 0.00) ppm using the residual proton solvent peaks as internal standards. Coupling constants (J) are reported in Hertz (Hz) and the multiplicity is abbreviated as follows: s (singlet), d (doublet), dd (doublet of doublet), t (triplet), m (multiplet). ESI-MS experiments were carried out using a LTQ-Orbitrap XL from Thermo Scientific (Thermo Fisher Scientific) and operated in positive ionization mode, with a spray voltage at 3.6 kV. No Sheath and auxiliary gas were used. Applied voltages were 40 and 100 V for the ion transfer capillary and the tube lens, respectively. The ion transfer capillary was held at 275° C. Detection was achieved in the Orbitrap with a resolution set to 100,000 (at m/z 400) and a m/z range between 150-2000 in profile mode. Spectrum was analyzed using the acquisition software XCalibur 2.1 (Thermo Fisher Scientific). The automatic gain control (AGC) allowed accumulation of up to 2.105 ions for FTMS scans, Maximum injection time was set to 300 ms and 1 μscan was acquired. 10 μL was injected using a Thermo Finnigan Surveyor HPLC system (Thermo Fisher Scientific) with a continuous infusion of methanol at 100 μL·min⁻¹. Elemental microanalyses were performed on a Thermo Flash 2000 elemental analyser. The absorption of the samples has been measured with a SpectraMax M2 Spectrometer (Molecular Devices). For analytic and preparative HPLC the following system has been used: 2× Agilent G1361 1260 Prep Pump system with Agilent G7115A 1260 DAD WR Detector equipped with an Agilent Pursuit XRs 5C18 (Analytic: 100 Å, C18 5 μm 250×4.6 mm, Preparative: 100 Å, C18 5 μm 250×300 mm) Column and an Agilent G1364B 1260-FC fraction collector. The solvents (HPLC grade) were millipore water (0.1% TFA, solvent A) and acetonitrile (0.1% TFA, solvent B). Inductive coupled plasma mass spectrometry (ICP-MS) experiments were carried out on an iCAP RQ ICP-MS instrument (Thermo Fisher).

Synthesis of the Ligands

### (E,E′)-4,4′-Bisstyryl-2,2′-bipyridine

The synthesis of 4,4′-Bisstyryl-2,2′-bipyridine is already published (Meyer, T. J. et al., 1987) but in this study another synthetic route was employed. 4,4′-Dimethyl-2,2′-bipyridine (1000 mg, 5.43 mmol, 1.0 equiv.) was dissolved in dry DMF under nitrogen atmosphere and Benzaldehyde (1.2 mL, 11.84 mmol, 2.2 equiv.) was added to the solution. Afterwards potassium tert-butoxide (2436 mg, 21.72 mmol, 4.0 equiv.) was added slowly. The colour of the solution turned to green and the mixture was stirred for 24 h. After that the mixture was poured into H₂O (400 mL) and the suspension cooled down to 5° C. The crude product which precipitated, was filtered and washed with Methanol. The product was purified by recrystallization from boiling acetic acid. The obtained solid was dissolved in Dichloromethane and the mixture was washed with a 5% LiCl aqueous solution, brine and H₂O. The solvent was removed and the product was isolated by recrystallization from boiling acetic acid. 1292 mg of (E,E′)-4,4′-Bisstyryl-2,2′-bipyridine (3.58 mmol, 66%) were yielded as a beige solid.

### 4,4′-Bis[p-(N,N-dimethylamino)styryl]-2,2′-bipyridine

The synthesis of 4,4′-Bis[p-(N,N-dimethylamino)styryl]-2,2′-bipyridine is already published (Wu, Q. et al) but in this study another synthetic route was employed. 4,4′-Dimethyl-2,2′-bipyridine (1000 mg, 5.43 mmol, 1.0 equiv.) was dissolved in dry DMF (100 mL) under nitrogen atmosphere and potassium tert-butoxide (2437 mg, 21.72 mmol, 4.0 equiv.) was added slowly. After 1.5 h of stirring, 4-(Dimethylamino)benzaldehyde (1701 mg, 11.40 mmol, 2.1 equiv.) was added to the reaction mixture. The colour of the solution turned to yellow and the mixture was heated at 90° C. for 19 h. After that the mixture was poured into H₂O (400 mL) and the suspension cooled down to 5° C. The crude product which precipitated, was filtered and washed with H₂O and Et₂O. The product was isolated by recrystallization from DCM/Pentane. 1541 mg of (E,E′)-4,4′-Bis[p-(N,N-dimethylamino)styryl]-2,2′-bipyridine (3.45 mmol, 64%) were yielded as a yellow solid.

### (E,E′)-4,4′-Bis[p-methoxystyryl]-2,2′-bipyridine

The synthesis of (E,E′)-4,4′-Bis[p-methoxystyryl]-2,2′-bipyridine is already published (Wu, Q. et al) but in this study another synthetic route was employed. 4,4′-Dimethyl-2,2′-bipyridine (532 mg, 2.89 mmol, 1.0 equiv.) was dissolved in dry DMF (25 mL) under nitrogen atmosphere and 4-Methoxybenzaldehyde (0.88 mL, 7.22 mmol, 2.5 equiv.) was added to the solution. Afterwards potassium tert-butoxide (1360 mg, 12.13 mmol, 4.2 equiv.) was added slowly. The colour of the solution turned to green and the mixture was stirred for 24 h. After that the mixture which turned bright was poured into H₂O (200 mL) and the suspension cooled down to 5° C. The crude product which precipitated, was filtered and washed with Methanol. The product was purified by recrystallization from boiling acetic acid. The obtained solid was dissolved in Dichloromethane and the mixture was washed with a 5% LiCl aqueous solution, brine and H₂O. The solvent was removed and the product was isolated by recrystallization from boiling acetic acid. 925 mg of (E,E′)-4,4′-Bis[p-methoxystyryl]-2,2′-bipyridine (2.20 mmol, 76%) were yielded as a beige solid.

### (E,E′)-4,4′-Bis[m-methoxystyryl]-2,2′-bipyridine

(E,E′)-4,4′-Bis[m-methoxystyryl]-2,2′-bipyridine was synthesized according to the literature (Aranyos, V. et al., 2001).

### (E,E′)-4,4′-Bis[o-methoxystyryl]-2,2′-bipyridine

(E,E′)-4,4′-Bis[o-methoxystyryl]-2,2′-bipyridine was synthesized according to the literature (Sinha, S., 2015)

### (E,E′)-4,4′-Bis[p-fluorostyryl]-2,2′-bipyridine

4,4′-Dimethyl-2,2′-bipyridine (1.00 g, 5.43 mmol, 1.0 equiv.) was dissolved in dry DMF (50 mL) under nitrogen atmosphere and 4-fluorobenzaldehyde (1.46 mL, 13.57 mmol, 2.5 equiv.) was added to the solution. Afterwards potassium tert-butoxide (2.44 g, 21.74 mmol, 4.0 equiv.) was added slowly. The colour of the solution turned to green and the mixture was stirred for 24 h. After that the mixture which turned bright was poured into H₂O (500 mL) and the suspension cooled down to 5° C. The crude product which precipitated, was filtered, and washed with methanol. The product was purified by recrystallization from boiling acetic acid. The obtained solid was dissolved in Dichloromethane and the mixture was washed with a 5% LiCl aqueous solution, brine, and H₂O. The solvent was removed to yield (E,E′)-4,4′-Bis[p-fluorostyryl]-2,2′-bipyridine as a beige solid (1.38 g, 3.48 mmol, 64%). ¹H-NMR (400 MHz, CD₂Cl₂): δ 8.75 (dd, J=5.0, 0.7 Hz, 2H), 8.68 (dd, J=1.7, 0.7 Hz, 2H), 7.69 (dd, J=8.7, 5.3 Hz, 4H), 7.55 (d, J=16.5 Hz, 2H), 7.52 (dd, J=5.0, 1.7 Hz, 2H), 7.22 (dd, J=8.7, 5.3 Hz, 4H), 7.20 (d, J=16.5 Hz, 2H); ESI-HRMS (pos. detection mode): calcd for C₂₆H₁₉N₂F₂, 397.1516; found, 397.1513.

### (E,E′)-4,4′-Bis[p-hydroxystyryl]-2,2′-bipyridine

(E,E′)-4,4′-Bis[p-hydroxystyryl]-2,2′-bipyridine was synthesized according to the literature (n K. K. et al., 1995).

### (E,E′)-4,4′-Bis[p-nitrostyryl]-2,2′-bipyridine

(E,E′)-4,4′-Bis[p-nitrostyryl]-2,2′-bipyridine was synthesized according to the literature (K. K. et al., 1995).

### (E,E′)-4,4′-Bis[p-aminostyryl]-2,2′-bipyridine

(E,E′)-4,4′-Bis[p-aminostyryl]-2,2′-bipyridine was synthesized according to the literature (Gajardo, F. et al., 2011).

### Tetraethyl 2,2′-bipyridine-4,4′-bisphosphonate

Tetraethyl-2,2′-bipyridine-4,4′-bisphosphonate was synthesized according to the literature (Woo, S. J., 2019).

### 4,4′-bis((E)-2-(4-methoxyphenyl)prop-1-en-1-yl)-2,2′-bipyridine

Under nitrogen, tetraethyl-2,2′-bipyridine-4,4′-bisphosphonate (200 mg, 0.44 mmol, 1.0 equiv.) was placed in a flame-dried flask. Anhydrous THF (10 mL) was added and the solution was cooled down to 0° C. KHMDS (2.6 mL, 1.30 mmol, 3.0 equiv., 0.5 mol/L solution in toluene) was added and the mixture was allowed to warm up to room temperature. The mixture was heated at 60° C. for 1 h. The mixture was then cooled down and 4-acetanisole (330 mg, 2.20 mmol, 5.0 equiv.) dissolved in anhydrous THF (5 mL) was added. The mixture was heated at 60° C. for 15 h. The mixture was cooled down and distilled water (35 mL) was added. The suspension was centrifuged, the solid was washed with hot ethanol and dried under vacuum to yield a beige powder (65 mg, 0.14 mmol, 32%). ¹H-NMR (400 MHz, CDCl₃): δ 8.66 (d, J=5.1 Hz, 2H), 8.39 (s, 2H), 7.49 (d, J=8.8 Hz, 4H), 7.28 (d, J=4.4 Hz, 2H), 6.93 (d, J=8.8 Hz, 4H), 6.79 (s, 2H), 3.85 (s, 6H), 2.35 (d, J=1.32, 6H). ¹³C-NMR (CDCl₃, 125 MHz): δ=159.6, 156.4, 149.2, 147.3, 141.1, 135.8, 127.4, 124.1, 123.8, 121.5, 114.0, 55.5, 18.0. ESI-HRMS (pos. detection mode): calcd for C₃₀H₂₉N₂O₂ m/z [M+H]+ 449.2229; found: 449.2223.

Synthesis of the Ruthenium Complexes

### [Ru(E,E′)-4,4′-Bisstyryl-2,2′-bipyridine)₃][PF₆]₂ (1)

(E,E′)-4,4′-Bisstyryl-2,2′-bipyridine (400 mg, 1.11 mmol, 4.0 equiv.) and Ru(DMSO)₄Cl₂ (134 mg, 0.28 mmol, 1.0 equiv.) were suspended in dry Ethanol (150 mL) under nitrogen atmosphere and the mixture was refluxed for 24 h. Then the solution was cooled down and undissolved residue was removed via filtration. To the residual solution a sat. aqueous solution of NH₄PF₆ was added. The crude product, which precipitated as a PF₆ salt was collected by centrifugation and washed with Ethanol, H₂O and Et₂O. The crude product was dissolved in Dichloromethane and washed with a 5% LiCl aqueous solution, brine and H₂O. After drying, 323 mg of 1 (0.22 mmol, 79%) were yielded as a red solid. ¹H-NMR (CD₃CN, 500 MHz): δ=8.76 (d, ³J=1.8 Hz, 6H), 7.78 (d, 3l=16.4 Hz, 6H), 7.76 (d, ³J=5.9 Hz, 6H), 7.71-7.69 (m, 12H), 7.51 (dd, ^{3,4}J=5.9, 1.8 Hz, 6H), 7.49-7.46 (m, 12H), 7.44-7.40 (m, 6H), 7.33 (d, ³J=16.4 Hz, 6H). ¹³C-NMR (CD₃CN, 125 MHz): δ=158.2, 152.4, 147.6, 137.3, 136.8, 130.6, 130.1, 128.4, 125.4, 125.1, 121.7. ESI-HRMS (pos. detection mode): calcd for C78H60N6Ru m/z [M]²⁺ 591.1956; found: 591.1978. Elemental analysis calcd for C78H60F12N6P2Ru+4*H2O (%): C, 60.66, H, 4.44, N, 5.44; found: C, 60.62, H, 4.43, N, 5.86. [Ru(E,E′)-4,4′-Bisstyryl-2,2′-bipyridine)₃][Cl]₂: The counter ion PF₆ was exchanged to Cl by elution with MeOH from the ion exchange resin Amberlite IRA-410. Elemental analysis calcd for C78H60Cl2N6Ru (%): C, 74.75, H, 4.83, N, 6.71; found: C, 74.36, H, 4.51, N, 6.37.

### [Ru((E,E′)-4,4′-Bis[p-(N,N-dimethylamino)styryl]-2,2′-bipyridine)₃][PF₆]₂ (2)

(E,E′)-4,4′-Bis[p-(N,N-dimethylamino)styryl]-2,2′-bipyridine (338 mg, 0.76 mmol, 4.0 equiv.), Ru(DMSO)₄Cl₂ (92 mg, 0.19 mmol, 1.0 equiv.) and LiCl (401 mg, 9.46 mmol, 50.0 equiv.) were dissolved in dry DMF (50 mL) under nitrogen atmosphere. The mixture was refluxed for 48 h. The solution was then cooled down and a sat. aqueous solution of NH₄PF₆ was added. The crude product, which precipitated as a PF₆ salt was collected by centrifugation and washed with Ethanol, H₂O and Et₂O. The residue was dissolved in Dichloromethane and washed with a 5% LiCl aqueous solution, brine and H₂O. The solvent was removed under reduced pressure and the crude product recrystallized from DCM/Pentane. The product was isolated via fractionated precipitation from CH₃CN by adding dropwise Et₂O. 86 mg of 2 (0.05 mmol, 26%) were yielded as a black solid. ¹H-NMR (CD₃CN, 500 MHz): δ=8.62 (d, ³J=1.9 Hz, 6H), 7.66 (d, ³J=16.2 Hz, 6H), 7.65 (d, ³J=6.1 Hz, 6H), 7.55-7.52 (m, 12H), 7.38 (dd, ^{3,3}J=6.1, 1.9 Hz, 6H), 7.03 (d, ³J=16.2 Hz, 6H), 6.81-6.78 (m, 12H), 3.01 (s, 36H). ¹³C-NMR (CD₃CN, 125 MHz): δ=159.4, 158.1, 152.6, 148.3, 137.7, 130.0, 124.5, 124.4, 120.6, 119.7, 113.2, 40.4. ESI-HRMS (pos. detection mode): calcd for C90H90N12Ru m/z [M]²⁺ 720.3222; found: 720.3247. Elemental analysis calcd for C90H90F12N12P2Ru (%): C, 62.46, H, 5.24, N, 9.71; found: C, 62.54, H, 5.17, N, 9.79. [Ru((E,E′)-4,4′-Bis[p-(N,N-dimethylamino)styryl]-2,2′-bipyridine)₃][Cl]₂: The counter ion PF₆ was exchanged to Cl by elution with MeOH from the ion exchange resin Amberlite IRA-410. Elemental analysis calcd for C90H90Cl2N12Ru (%): C, 71.51, H, 6.00, N, 11.12; found: C, 71.19, H, 5.93, N, 10.84.

### [Ru((E,E′)-4,4′-Bis[p-methoxystyryl]-2,2′-bipyridine)₃][PF₆]₂ (3)

(E,E′)-4,4′-Bis[p-methoxystyryl]-2,2′-bipyridine (286 mg, 0.68 mmol, 4.0 equiv.) and Ru(DMSO)₄Cl₂ (82 mg, 0.17 mmol, 1.0 equiv.) were suspended dry EtOH (50 mL) under nitrogen atmosphere. The mixture was refluxed for 15 h. The solution was then cooled down and undissolved solid was removed by filtration. A sat. aqueous solution of NH₄PF₆ was added and the crude product, which precipitated as a PF6 salt was collected by filtration. The solid was washed with H₂O and Et₂O. The residue was purified via fractionated precipitation from CH₃CN by adding dropwise Et₂O. The collected product was dissolved in Dichloromethane and washed with a 5% LiCl aqueous solution, brine and H₂O. After drying, 218 mg of 3 (0.13 mmol, 76%) were yielded as a black solid. ¹H-NMR (CD₃CN, 500 MHz): δ=8.79 (d, ⁴J=1.7 Hz, 6H), 7.79 (d, ³J=16.4 Hz, 6H), 7.71 (d, ³J=6.0 Hz, 6H), 7.64 (d, ³J=8.9 Hz, 12H), 7.44 (d, ^{3,4}J=6.0, 1.7 Hz, 6H), 7.17 (d, ³J=16.4 Hz, 6H), 7.00 (d, ³J=8.9 Hz, 12H), 3.83 (s, 18H). ¹³C-NMR (CD₃CN, 125 MHz): δ=162.0, 158.2, 152.2, 148.0, 137.0, 130.1, 129.5, 125.0, 122.7, 121.3, 115.5, 56.2. ESI-HRMS (pos. detection mode): calcd for C84H72N6O6Ru m/z [M]²⁺ 681.2290; found: 681.2273. Elemental analysis calcd for C84H72F12N6O6P2Ru (%): C, 61.05, H, 4.39, N, 5.09; found: C, 61.17, H, 4.44, N, 5.21. [Ru((E,E′)-4,4′-Bis[p-methoxystyryl]-2,2′-bipyridine)₃][Cl]₂: The counter ion PF₆ was exchanged to Cl by elution with MeOH from the ion exchange resin Amberlite IRA-410. Elemental analysis calcd for C84H72Cl₂N6O6Ru (%): C, 70.38, H, 5.06, N, 5.86; found: C, 70.62, H, 5.28, N, 5.57.

### [Ru(bipy)((E,E′)-4,4′-Bis[p-(N,N-dimethylamino)styryl]-2,2′-bipyridine)₂][PF₆]₂ (4)

(E,E′)-4,4′-Bis[p-(N,N-dimethylamino)styryl]-2,2′-bipyridine (220 mg, 0.49 mmol, 2.0 equiv.), Ru(DMSO)₄Cl₂ (119 mg, 0.25 mmol, 1.0 equiv.) and LiCl (1044 mg, 24.63 mmol, 100 equiv.) were suspended in dry DMF (30 mL) under nitrogen atmosphere. The mixture was refluxed for 4 h. The solution was then cooled down and H₂O was added. The crude product, which precipitated was collected by filtration and washed with H₂O and Et₂O. The formation of [Ru((E,E′)-4,4′-Bis[p-(N,N-dimethylamino)styryl]-2,2′-bipyridine)₂Cl₂] was analysed via HPLC. [Ru((E,E′)-4,4′-Bis[p-(N,N-dimethylamino)styryl]-2,2′-bipyridine)₂C₂] and 2,2′-Bipyridine (47 mg, 0.3 mmol, 1.2 equiv.) were suspended in dry Ethanol (50 mL) under nitrogen atmosphere. The mixture was refluxed for 7 h. The solution was then cooled down and undissolved solid was removed by filtration. A sat. aqueous solution of NH₄PF₆ was added and the crude product, which precipitated as a PF₆ salt was collected by filtration. The solid was washed with H₂O and Et₂O. The residue was purified via preparative HPLC as a TFA salt. The solvents were millipore water (0.1% TFA, solvent A) and acetonitrile (solvent B). The following HPLC gradient has been used: 0-3 minutes: isocratic 50% A (50% B); 3-17 minutes: linear gradient from 50% A (50% B) to 0% A (100% B); 17-23 minutes: isocratic 0% A (100% B). The flow rate was 20 mL/min and the chromatogram was detected at 250 nm, 350 nm, 450 nm. The collected product was dissolved in CH₃CN and a sat. aqueous solution of NH₄PF₆ was added. The product, which precipitated as a PF6 salt was collected by filtration and washed with H₂O, Et₂O and Pentane. 89 mg of 4 (0.06 mmol, 24%) were yielded as a dark red solid. ¹H-NMR (CD₃CN, 400 MHz): δ=8.61 (s, 4H), 8.50 (d, J=8.2 Hz, 2H), 8.04 (td, J=8.0, 1.5 Hz, 2H), 7.86 (ddd, J=5.7, 1.4, 0.6 Hz, 2H), 7.66 (dd, J=16.2, 1.9 Hz, 4H), 7.64 (d, J=6.3 Hz, 2H), 7.56-7.50 (m, 10H), 7.43-7.34 (m, 6H), 7.02 (dd, J=16.2 Hz, 4H), 6.81-6.77 (m, 8H), 3.02 (s, 12H), 3.01 (s, 12H). ¹³C-NMR (CD₃CN, 100 MHz): δ=158.1, 158.0, 152.7, 152.6, 151.9, 151.9, 148.5, 138.3, 137.9, 130.0, 128.4, 125.1, 124.4, 120.7, 119.6, 113.2, 40.4. ESI-HRMS (pos. detection mode): calcd for C70H68N10Ru m/z [M]²⁺ 575.2330; found: 575.2347. Elemental analysis calcd for C70H68F12N10P2Ru (%): C, 58.37, H, 4.76, N, 9.72; found: C, 58.19, H, 4.62, N, 9.72.

### [Ru(bipy)((E,E′)-4,4′-Bis[p-methoxystyryl]-2,2′-bipyridine)₂][PF₆]₂ (5)

(E,E′)-4,4′-Bis[p-methoxystyryl]-2,2′-bipyridine (490 mg, 1.17 mmol, 2.0 equiv.), Ru(DMSO)₄Cl₂ (282 mg, 0.58 mmol, 1.0 equiv.) and LiCl (2470 mg, 58.26 mmol, 100 equiv.) were suspended in dry DMF (75 mL) under nitrogen atmosphere. The mixture was refluxed for 6 h. The solution was then cooled down and purged into H₂O. The crude product, which precipitated was collected by filtration and washed with H₂O and Et₂O. The formation of [Ru((E,E′)-4,4′-Bis[p-methoxystyryl]-2,2′-bipyridine)₂C₂] was analysed via HPLC. [Ru((E,E′)-4,4′-Bis[p-methoxystyryl]-2,2′-bipyridine)₂Cl₂] and 2,2′-Bipyridine (109 mg, 0.70 mmol, 1.2 equiv.) were suspended in dry Ethanol (100 mL) under nitrogen atmosphere. The mixture was refluxed for 6 h. The solution was then cooled down and undissolved solid was removed by filtration. A sat. aqueous solution of NH₄PF₆ was added and the crude product, which precipitated as a PF₆ salt was collected by centrifugation. The solid was washed with H₂O and Et₂O. The residue was purified via preparative HPLC as a TFA salt. The solvents were millipore water (0.1% TFA, solvent A) and acetonitrile (solvent B). The following HPLC gradient has been used: 0-3 minutes: isocratic 50% A (50% B); 3-17 minutes: linear gradient from 50% A (50% B) to 0% A (100% B); 17-23 minutes: isocratic 0% A (100% B). The flow rate was 20 mL/min and the chromatogram was detected at 250 nm, 350 nm, 450 nm. The collected product was dissolved in CH₃CN and a sat. aqueous solution of NH₄PF₆ was added. The product, which precipitated as a PF6 salt was collected by filtration and washed with H₂O, Et₂O and Hexane. 248 mg of 5 (0.18 mmol, 31%) were yielded as a dark red solid. ¹H-NMR (CD₃CN, 400 MHz): δ=8.72 (d, J=1.6 Hz, 4H), 8.51 (d, J=8.2 Hz, 2H), 8.06 (td, J=8.0, 1.3 Hz, 2H), 7.85 (dd, J=5.6, 1.1 Hz, 2H), 7.75-7.68 (m, 6H), 7.67-7.61 (m, 8H), 7.60 (d, J=5.9 Hz, 2H), 7.46-7.39 (m, 6H), 7.17 (dd, J=16.4, 2.1 Hz, 4H), 7.05-6.99 (m, 8H), 3.85 (s, 6H), 3.84 (s, 6H). ¹³C-NMR (CD₃CN, 100 MHz): δ=162.0, 158.1, 152.6, 152.2, 148.1, 138.6, 137.1, 132.8, 130.1, 129.5, 128.5, 125.2, 125.0, 122.7, 121.3, 115.5, 56.2. ESI-HRMS (pos. detection mode): calcd for C66H56N6O4Ru m/z [M]²⁺ 549.1698; found: 549.1707. Elemental analysis calcd for C66H56F12N6O4P2Ru+1Hexane (%): C, 57.96, H, 4.44, N, 5.87; found: C, 58.43, H, 3.94, N, 5.79.

### [Ru(bipy)₂((E,E′)-4,4′-Bis[p-(N,N-dimethylamino)styryl]-2,2′-bipyridine)][PF₆]₂ (6)

Ru(bipy)₂Cl₂ (350 mg, 0.72 mmol, 1.0 equiv.) and (E,E′)-4,4′-Bis[p-(N,N-dimethylamino)styryl]-2,2′-bipyridine (388 mg, 0.87 mmol, 1.2 equiv.) were suspended in dry Ethanol (50 mL) under nitrogen atmosphere and the mixture was refluxed for 6 h. Then the solution was cooled down and a sat. aqueous solution of NH₄PF₆ was added. The crude product, which precipitated as a PF₆ salt was collected by filtration and washed with H₂O and Et₂O. The product was isolated via fractionated precipitation from CH₃CN by adding dropwise Et₂O. 449 mg of 6 (0.39 mmol, 54%) were yielded as a dark red solid. ¹H-NMR (CD₃CN, 500 MHz): δ=8.62 (d, J=1.7 Hz, 2H), 8.50 (d, J=8.2 Hz, 4H), 8.07-8.02 (m, 4H), 7.87-7.84 (m, 2H), 7.75-7.72 (m, 2H), 7.67 (d, J=16.3 Hz, 2H), 7.57-7.52 (m, 4H), 7.52-7.49 (d, J=6.0 Hz, 2H), 7.44-7.34 (m, 6H), 7.02 (d, J=16.3 Hz, 2H), 6.81-6.76 (m, 4H), 3.01 (s, 12H). ¹³C-NMR (CD₃CN, 125 MHz): 5=158.1, 158.0, 158.0, 152.7, 152.7, 152.6, 151.9, 148.8, 138.6, 138.0, 130.0, 128.5, 128.5, 125.2, 124.4, 124.3, 120.8, 119.5, 113.1, 40.4. ESI-HRMS (pos. detection mode): calcd for C50H46N8Ru m/z [M]²⁺ 430.1439; found: 430.1441. Elemental analysis calcd for C50H46F12N8P2Ru (%): C, 52.22, H, 4.03, N, 9.74; found: C, 51.97, H, 4.04, N, 9.71.

### [Ru(bipy)₂((E,E′)-4,4′-Bis[p-(N,N-methoxy)styryl]-2,2′-bipyridine)][PF₆]₂ (7)

Ru(bipy)₂Cl₂ (432 mg, 0.89 mmol, 1.0 equiv.) and (E,E′)-4,4′-Bis[p-(N,N-methoxy)styryl]-2,2′-bipyridine (450 mg, 1.07 mmol, 1.2 equiv.) were suspended in dry Ethanol (100 mL) under nitrogen atmosphere and the mixture was refluxed for 6 h. Then the solution was cooled down and a sat. aqueous solution of NH₄PF₆ was added. The crude product, which precipitated as a PF₆ salt was collected by filtration and washed with H₂O and Et₂O. The product was isolated via fractionated precipitation from CH₃CN by adding dropwise Et₂O. 358 mg of 7 (0.32 mmol, 36%) were yielded as a dark red solid. ¹H-NMR (CD₃CN, 500 MHz): δ=8.71 (d, J=1.4 Hz, 2H), 8.51 (dd, J=8.2, 0.7 Hz, 4H), 8.06 (td, J=8.0, 1.5 Hz, 4H), 7.86-7.84 (m, 2H), 7.76-7.73 (m, 2H), 7.72 (d, J=16.4 Hz, 2H), 7.66-7.62 (m, 4H), 7.59 (d, J=6.0 Hz, 2H), 7.45-7.38 (m, 6H), 7.16 (d, J=16.4 Hz, 2H), 7.03-6.98 (m, 4H), 3.83 (s, 6H). ¹³C-NMR (CD₃CN, 125 MHz): δ=162.0, 158.1, 158.0, 152.7, 152.6, 152.2, 148.2, 138.7, 138.7, 137.1, 130.1, 129.5, 128.6, 128.5, 125.2, 125.0, 122.6, 121.4, 115.5, 56.2. ESI-HRMS (pos. detection mode): calcd for C48H40N6O2Ru m/z [M]²⁺ 417.1123; found: 417.1126. Elemental analysis calcd for C48H40F12N6O2P2Ru (%): C, 51.30, H, 3.59, N, 7.48; found: C, 51.23, H, 3.48, N, 7.61. [Ru(bipy)₂((E,E′)-4,4′-Bis[p-(N,N-methoxy)styryl]-2,2′-bipyridine)][Cl]₂: The counter ion PF₆ was exchanged to Cl by elution with MeOH from the ion exchange resin Amberlite IRA-410. Elemental analysis calcd for C48H40Cl₂N6O2Ru (%): C, 63.70, H, 4.46, N, 9.29; found: C, 63.51, H, 4.30, N, 9.11.

### [Ru(phen)₂((E,E′)-4,4′-Bis[p-(N,N-dimethylamino)styryl]-2,2′-bipyridine)][PF₆]₂ (8)

Ru(phen)₂Cl₂ (455 mg, 0.86 mmol, 1.0 equiv.) and (E,E′)-4,4′-Bis[p-(N,N-dimethylamino)styryl]-2,2′-bipyridine (458 mg, 1.03 mmol, 1.2 equiv.) were suspended in dry Ethanol (50 mL) under nitrogen atmosphere and the mixture was refluxed for 19 h. Then the solution was cooled down, undissolved residue was removed via filtration and washed with Ethanol. To the residual solution a sat. aqueous solution of NH₄PF₆ was added. The crude product, which precipitated as a PF₆ salt was collected by filtration and washed with water and Et₂O. The solid was dissolved in Dichloromethane and washed with a 5% LiCl aqueous solution, brine and water. The solvent was removed under reduced pressure and the product was purified via fractionated precipitation from Acetonitrile by adding dropwise Et₂O. The obtained solid was separated by filtration and was washed with H₂O, Et₂O and Pentane. 427 mg of 1 (0.36 mmol, 41%) were yielded as a red solid. ¹H-NMR (CD₃CN, 400 MHz): δ=8.68-8.63 (m, 4H), 8.54 (dd, J=8.3, 1.4 Hz, 2H), 8.34-8.31 (m, 2H), 8.29-8.14 (m, 6H), 7.89 (dd, J=5.4, 1.4 Hz, 2H), 7.81 (dd, J=8.3, 5.1 Hz, 2H), 7.64 (d, J=16.4 Hz, 2H), 7.57-7.49 (m, 4H), 7.46 (d, J=6.1 Hz, 2H), 7.24 (dd, J=6.0, 1.7 Hz, 2H), 7.00 (d, J=16.4 Hz, 2H), 6.80-6.74 (m, 4H), 3.00 (s, 12H). ¹³C-NMR (CD₃CN, 100 MHz): δ=158.3, 153.7, 153.6, 152.7, 152.4, 148.9, 148.7, 137.9, 137.6, 137.5, 132.0, 130.0, 129.0, 127.0, 126.8, 124.3, 124.2, 120.7, 119.5, 113.7, 113.1, 40.4. HR-MS (pos. detection mode): calcd for C₅₄H₄₆N₈Ru m/z [M]²⁺ 454.1439; found: 454.1455. Elemental analysis calcd for C54H₄₆F₁₂N₈P₂Ru (%): C, 54.21, H, 3.91, N, 9.24; found: C, 54.14, H, 3.87, N, 9.35.

### [Ru(phen)₂((E,E′)-4,4′-Bis[p-methoxystyryl]-2,2′-bipyridine)][PF₆]₂ (9)

Ru(phen)₂Cl₂ (443 mg, 0.83 mmol, 1.0 equiv.) and (E,E′)-4,4′-Bis[p-methoxystyryl]-2,2′-bipyridine (420 mg, 0.99 mmol, 1.2 equiv.) were suspended in dry Ethanol (50 mL) under nitrogen atmosphere and the mixture was refluxed for 24 h. Then the solution was cooled down and undissolved residue was removed via filtration. To the residual solution a sat. aqueous solution of NH₄PF₆ was added. The crude product, which precipitated as a PF₆ salt was collected by filtration and washed with water and Et₂O. The product was isolated by column chromatography on silica gel with an Acetonitrile/aq. KNO₃ (0.4 M) solution (10:1). The fractions containing the product were united and the solvent was removed under reduced pressure. The residue was dissolved in Acetonitrile and undissolved KNO₃ was removed by filtration. The solvent was removed again and the product was dissolved in H₂O (50 mL). Upon addition of NH₄PF₆ the product precipitated as a PF₆ salt. The solid was obtained by filtration and was washed three-times with H₂O and Et₂O. 672 mg of 2 (0.57 mmol, 69%) were yielded as a red solid. ¹H-NMR (CD₃CN, 400 MHz): δ=8.70 (s, 2H), 8.65 (d, J=8.2 Hz, 2H), 8.55 (d, J=8.2 Hz, 2H), 8.32-8.22 (m, 6H), 7.89 (d, J=4.7 Hz, 2H), 7.81 (dd, J=8.2, 5.2 Hz, 2H), 7.68 (d, J=16.2 Hz, 2H), 7.63-7-53 (m, 8H), 7.30 (d, J=5.5 Hz, 2H), 7.13 (d, J=16.2 Hz, 2H), 7.01 (d, J=8.0 Hz, 4H), 3.83 (s, 6H). ¹³C-NMR (CD₃CN, 100 MHz): δ=16 2.0, 158.4, 153.7, 153.6, 15 2.7, 148.9, 148.6, 148.1, 137.7, 137.6, 137.0, 13 2.0, 130.0, 129.4, 129.0, 127.0, 126.8, 124.7, 122.6, 121.2, 115.5, 56.1. HR-MS (pos. detection mode): calcd for C₅₂H₄₀N₆O₂Ru m/z [M]²⁺ 441.1123; found: 441.1131. Elemental analysis calcd for C52H₄₀F₁₂N₆O₂P₂Ru (%): C, 53.29, H, 3.44, N, 7.17; found: C, 53.18, H, 3.35, N, 7.26.

### [Ru(4,7-Diphenyl-1,10-phenanthroline)₂((E,E′)-4,4′-Bis[p-(N,N-dimethylamino)styryl]-2,2′-bipyridine)][PF₆]₂ (10)

Ru(4,7-Diphenyl-1,10-phenanthroline)₂Cl₂ (335 mg, 0.40 mmol, 1.0 equiv.) and (E,E′)-4,4′-Bis[p-(N,N-dimethylamino)styryl]-2,2′-bipyridine (215 mg, 0.48 mmol, 1.2 equiv.) were suspended in dry Ethanol (100 mL) under nitrogen atmosphere and the mixture was refluxed for 24 h. Then the solution was cooled down and undissolved residue was removed via filtration. The residual solution was diluted with a mixture of Ethanol and water and a sat. aqueous solution of NH₄PF₆ was added. The crude product, which precipitated as a PF₆ salt was collected by centrifugation and washed with water and Et₂O. The solid was dissolved in Acetonitrile and undissolved residue was removed via filtration. The solvent was removed under reduced pressure and the obtained solid was dissolved in Dichloromethane and washed with a 5% LiCl aqueous solution, brine and water. The solvent was removed under reduced pressure. The product was isolated via fractionated precipitation from Methanol by adding dropwise Et₂O. After drying, 289 mg of 3 (0.19 mmol, 48%) were yielded as a red solid. ¹H-NMR (CD₃CN, 400 MHz): δ=8.73 (d, J=1.5 Hz, 2H), 8.42 (d, J=5.5 Hz, 2H), 8.26-8.16 (m, 4H), 8.12 (d, J=5.5 Hz, 2H), 7.78 (d, J=5.5 Hz, 2H), 7.70 (d, J=16.3 Hz, 2H), 7.67-7.57 (m, 24H), 7.54 (d, J=8.9 Hz, 4H), 7.34 (dd, J=6.1, 1.7 Hz, 2H), 7.05 (d, J=16.3 Hz, 2H), 6.78 (d, J=9.0 Hz, 4H), 3.01 (s, 12H). ¹³C-NMR (CD₃CN, 100 MHz): δ=158.3, 153.1, 152.7, 152.4, 149.9, 149.8, 149.5, 149.4, 148.8, 138.0, 136.8, 136.7, 130.8, 130.8, 130.6, 130.1, 130.1, 130.0, 129.9, 127.2, 127.0, 127.0, 127.0, 124.3, 120.8, 119.5, 113.1, 40.4. HR-MS (pos. detection mode): calcd for C₇₈H₆₂N₈Ru m/z [M]²⁺ 606.2065; found: 606.2078. Elemental analysis calcd for C₇₈H₆₂N₈RuP₂F₁₂+1.5 MeOH (%): C, 61.59, H, 4.42, N, 7.23; found: C, 61.73, H, 4.48, N, 6.88.

### [Ru(4,7-Diphenyl-1,10-phenanthroline)₂((E,E′)-4,4′-Bis[p-methoxystyryl]-2,2′-bipyridine)][PF₆]₂ (11)

Ru(4,7-Diphenyl-1,10-phenanthroline)₂Cl₂ (300 mg, 0.36 mmol, 1.0 equiv.) and (E,E′)-4,4′-Bis[p-methoxystyryl]-2,2′-bipyridine (181 mg, 0.43 mmol, 1.2 equiv.) were suspended in dry Ethanol (50 mL) under nitrogen atmosphere and the mixture was refluxed for 24 h. Then the solution was cooled down and undissolved residue was removed via filtration. The residual solution was diluted with a mixture of Ethanol and water and a sat. aqueous solution of NH₄PF₆ was added. The crude product, which precipitated as a PF₆ salt was collected by filtration and washed with water and Et₂O. The solid was dissolved in Dichloromethane and washed with a 5% LiCl aqueous solution, brine and water. The solvent was removed under reduced pressure. After drying, 395 mg of 4 (0.27 mmol, 74%) were yielded as a red solid. ¹H-NMR (CD₃CN, 400 MHz): δ=8.78 (s, 2H), 8.41 (d, J=5.5 Hz, 2H), 8.21 (s, 4H), 8.13 (d, J=5.5 Hz, 2H), 7.78 (d, J=5.5 Hz, 2H), 7.75 (d, J=2.3 Hz, 2H), 7.72 (d, J=8.0 Hz, 2H), 7.66-7.58 (m, 26H), 7.41 (d, J=6.1 Hz, 2H), 7.19 (d, J=16.4 Hz, 2H), 7.02 (d, J=8.7 Hz, 4H), 3.84 (s, 6H). ¹³C-NMR (CD₃CN, 100 MHz): δ=162.0, 158.4, 153.1, 152.8, 150.0, 149.9, 149.5, 149.3, 148.2, 137.1, 136.8, 136.7, 130.8, 130.8, 130.7, 130.1, 130.1, 130.1, 130.0, 129.9, 129.4, 127.2, 127.0, 124.9, 122.7, 121.3, 115.5, 56.2. HR-MS (pos. detection mode): calcd for C₇₆H₅₆N₆O₂Ru m/z [M]²⁺ 593.1749; found: 593.1768. Elemental analysis calcd for C₇₆H₅₆F₁₂N₆O₂P₂Ru+4*H₂O (%): C, 58.95, H, 4.17, N, 5.43; found: C, 58.58, H, 3.94, N, 5.72. [Ru(1,10-phenanthroline)₃][PF₆]₂([Ru(phen)₃][PF₆]2) (Comparative example) [Ru(phen)₃][PF₆]₂ was synthesized as previously published (Zuloaga, F. and Kasha, M., 1968) using RuCl₂(DMSO)₄ precursor. Purity of the sample was assessed by HPLC and elemental analysis. Elemental analysis calcd for C₃₆H₂₄F₁₂N₆P₂Ru (%): C, 46.41, H, 2.60, N, 9.02; found: C, 46.34, H, 2.54, N, 8.83.

### [Ru(bpy)₂((E,E′)-4,4′-Bis[m-methoxystyryl]-2,2′-bipyridine)][PF₆]₂ (12)

Under nitrogen, Ru(bpy)₂Cl₂ (200 mg, 0.41 mmol, 1.0 equiv.) and (E,E′)-4,4′-Bis[m-methoxystyryl]-2,2′-bipyridine (208 mg, 0.50 mmol, 1.2 equiv.) were suspended in dry ethanol (50 mL) and the mixture was refluxed for 6 h. Then the solution was cooled down and a sat. aqueous solution of NH₄PF₆ was added. The crude product, which precipitated as a PF₆ salt was collected by filtration and washed with water and diethyl ether. The product was purified by silica gel chromatography using acetonitrile/potassium nitrate 0.3 M in water (9:1 v/v) as eluent. The fractions containing the product were collected and the solvent evaporated. The residue was dissolved in acetonitrile and filtered. The filtrate was concentrated to dryness under vacuum. The residue was dissolved in ethanol and a sat. aqueous solution of NH₄PF₆ was added. The precipitate was filtered, washed with water and diethyl ether, and dried under vacuum to yield 12 (390 mg, 0.35 mmol, 85%) as a dark red solid. ¹H-NMR (400 MHz, CD₃CN): δ 8.72 (d, J=1.9 Hz, 2H), 8.52 (d, J=8.2 Hz, 4H), 8.07 (td, J=7.9, 1.5, 4H), 7.85 (d, J=5.6 Hz, 2H), 7.78-7.61 (m, 6H), 7.54-7.09 (m, 14H), 6.95 (dd, J=7.3, 1.9, 2H), 3.84 (s, 6H). ¹³C-NMR (CDCl₃, 101 MHz): δ=161.2, 158.2, 158.0, 152.7, 152.4, 147.6, 138.8, 138.3, 137.2, 131.1, 128.6, 125.4, 125.3, 125.2, 121.7, 121.3, 116.4, 113.2, 56.0. ESI-HRMS (pos. detection mode): calcd for C₄₈H₄₀N₆O₂Ru m/z [M]²⁺ 417.1128; found: 417.1126. Elemental analysis calcd for C₄₈H₄₀F₁₂N₆O₂P₂Ru (%): C, 51.30, H, 3.59, N, 7.48; found: C, 51.06, H, 3.61, N, 7.38.

### [Ru(bpy)₂((E,E′)-4,4′-Bis[o-methoxystyryl]-2,2′-bipyridine)][PF₆]₂ (13)

Under nitrogen, Ru(bpy)₂Cl₂ (150 mg, 0.31 mmol, 1.0 equiv.) and (E,E′)-4,4′-Bis[o-methoxystyryl]-2,2′-bipyridine (156 mg, 0.37 mmol, 1.2 equiv.) were suspended in dry ethanol (50 mL) and the mixture was refluxed for 6 h. Then the solution was cooled down and a sat. aqueous solution of NH₄PF₆ was added. The crude product, which precipitated as a PF₆ salt was collected by filtration and washed with water and diethyl ether. The product was purified by silica gel chromatography using acetonitrile/potassium nitrate 0.3 M in water (9:1 v/v) as eluent. The fractions containing the product were collected and the solvent evaporated. The residue was dissolved in acetonitrile and filtered. The filtrate was concentrated to dryness under vacuum. The residue was dissolved in ethanol and a sat. aqueous solution of NH₄PF₆ was added. The precipitate was filtered, washed with water and diethyl ether, and dried under vacuum to yield 13 (225 mg, 0.20 mmol, 65%) as a dark red solid. 1H-NMR (400 MHz, CD₃CN): δ 8.71 (d, J=1.9 Hz, 2H), 8.59-8.33 (m, 4H), 8.07 (m, 4H), 7.95 (d, J=16.5 Hz, 2H), 7.84 (d, J=4.3 Hz, 2H), 7.74 (ddd, J=5.6, 1.5, 0.8 Hz, 2H), 7.68 (dd, J=7.7, 1.7 Hz, 2H), 7.61 (d, J=6.0 Hz, 2H), 7.42 (m, 10H), 7.06 (m, 4H), 3.94 (s, 6H). ¹³C-NMR (CDCl₃, 101 MHz): δ=158.9, 158.2, 158.0, 152.6, 152.2, 148.3, 138.7, 132.7, 132.0, 129.0, 128.5, 125.8, 125.4, 125.2, 125.0, 121.9, 121.8, 112.6, 56.3. ESI-HRMS (pos. detection mode): calcd for C₄₈H₄₀N₆O₂Ru m/z [M]²⁺ 417.1128; found: 417.1126. Elemental analysis calcd for C₄₈H₄₀F₁₂N₆O₂P₂Ru+H₂O (%): C, 50.49, H, 3.71, N, 7.36; found: C, 50.27, H, 3.41, N, 7.66.

### [Ru(bpy)₂((E,E′)-4,4′-Bis[p-fluorostyryl]-2,2′-bipyridine)][PF₆]₂ (14)

Under nitrogen, Ru(bpy)₂Cl₂ (242 mg, 0.50 mmol, 1.0 equiv.) and (E,E′)-4,4′-Bis[p-fluorostyryl]-2,2′-bipyridine (238 mg, 0.60 mmol, 1.2 equiv.) were suspended in dry ethanol (50 mL) and the mixture was refluxed for 6 h. Then the solution was cooled down and a sat. aqueous solution of NH₄PF₆ was added. The crude product, which precipitated as a PF₆ salt was collected by filtration and washed with water and diethyl ether. The product was purified by silica gel chromatography using acetonitrile/potassium nitrate 0.3 M in water (9:1 v/v) as eluent. The fractions containing the product were collected and the solvent evaporated. The residue was dissolved in acetonitrile and filtered. The filtrate was concentrated to dryness under vacuum. The residue was dissolved in ethanol and a sat. aqueous solution of NH₄PF₆ was added. The precipitate was filtered, washed with water and diethyl ether, and dried under vacuum to yield 14 (273 mg, 0.29 mmol, 57%) as a dark red solid. ¹H-NMR (CD₃CN, 400 MHz): δ=8.72 (d, J=1.9 Hz, 2H), 8.53 (d, J=8.2 Hz, 4H), 8.07 (t, J=7.9 Hz, 4H), 7.85 (d, J=5.6 Hz, 2H), 7.80-7.68 (m, 8H), 7.65 (d, J=6.0 Hz, 2H), 7.52-7.36 (m, 6H), 7.26 (d, J=16.5 Hz, 2H), 7.19 (d, J=8.8 Hz, 4H). ¹³C-NMR (CD₃CN, 101 MHz): δ=164.3 (d, ¹J_{C-F}=248 Hz), 158.2, 158.0, 158.0, 152.7, 152.4, 147.6, 138.8, 136.0, 133.4 (d, ⁴J_{C-F}=3 Hz), 130.5 (d, ³J_{C-F}=8 Hz), 128.6, 125.2, 125.0, 121.8, 116.9 (d, ²J_{C-F}=22 Hz). ¹⁹F NMR (376 MHz, CD₃CN) δ=−72.77 (d, J=706.7 Hz), −112.83. ESI-HRMS (pos. detection mode): calcd for C₄₆H₃₄N₆F₂Ru m/z [M]²⁺ 405.0928; found: 405.0927. Elemental analysis calcd for C₄₆H₃₄F₁₄N₆O₂P₂Ru+H₂O (%): C, 49.43, H, 3.25, N, 7.52; found: C, 49.76, H, 3.02, N, 7.47.

### [Ru(bpy)₂((E,E′)-4,4′-Bis[p-hydroxystyryl]-2,2′-bipyridine)][PF₆]₂ (15)

Under nitrogen, Ru(bpy)₂Cl₂ (150 mg, 0.31 mmol, 1.0 equiv.) and (E,E′)-4,4′-Bis[p-hydroxystyryl]-2,2′-bipyridine (145 mg, 0.37 mmol, 1.2 equiv.) were suspended in ethylene glycol (5 mL) and the mixture was heated at 130° C. for 24 h. Then the solution was cooled down and a sat. aqueous solution of NH₄PF₆ was added. The crude product, which precipitated as a PF₆ salt was collected by filtration and washed with water and diethyl ether and dried under vacuum to yield 15 (297 mg, 0.27 mmol, 87%) as a dark red solid. ¹H-NMR (CD₃CN, 400 MHz): δ=8.66 (d, J=1.9 Hz, 2H), 8.50 (d, J=8.1 Hz, 4H), 8.05 (td, J=8.0, 1.5 Hz, 4H), 7.84 (d, J=5.1 Hz, 2H), 7.74 (d, J=5.0 Hz, 2H), 7.68 (d, J=16.4 Hz, 2H), 7.61-7.51 (m, 6H), 7.46-7.35 (m, 8H), 7.12 (d, J=16.5 Hz, 2H), 6.90 (d, J=8.6 Hz, 4H). ¹³C-NMR (CD₃CN, 101 MHz): δ=159.5, 158.01, 157.95, 152.6, 152.1, 148.2, 138.6, 137.3, 130.2, 128.7, 128.5, 125.1, 124.9, 122.1, 121.2, 116.9. ESI-HRMS (pos. detection mode): calcd for C₄₆H₃₆N₆O₂Ru m/z [M]²⁺ 403.0972; found: 403.0970. Elemental analysis calcd for C₄₆H₃₆F₁₂N₆O₂P₂Ru+H₂O (%): C, 49.60, H, 3.44, N, 7.55; found: C, 49.46, H, 3.35, N, 7.31.

### [Os(bpy)₂((E,E′)-4,4′-Bis[p-methoxystyryl]-2,2′-bipyridine)][PF₆]₂ (16)

Under nitrogen, Os(bpy)₂Cl₂ (100 mg, 0.17 mmol, 1.0 equiv.) and (E,E′)-4,4′-Bis[p-methoxystyryl]-2,2′-bipyridine (84 mg, 0.20 mmol, 1.2 equiv.) were suspended in ethylene glycol (5 mL). The mixture was degassed by nitrogen bubbling for 15 min and heated at 130° C. for 24 h. Then the solution was cooled down and a sat. aqueous solution of NH₄PF₆ was added. The crude product, which precipitated as a PF₆ salt was collected by filtration and washed with water and diethyl ether and dried under vacuum to yield 20 (189 mg, 0.16 mmol, 94%) as a dark brown solid. ¹H-NMR (CD₃CN, 400 MHz): δ=8.64 (d, J=2.0 Hz, 2H), 8.49 (d, J=8.0 Hz, 4H), 7.86 (ddt, J=9.7, 8.2, 1.7 Hz, 4H), 7.74 (d, J=5.4 Hz, 2H), 7.71-7.59 (m, 8H), 7.50 (d, J=6.1 Hz, 2H), 7.38-7.26 (m, 6H), 7.16 (d, J=16.3 Hz, 2H), 7.02 (d, J=8.8 Hz, 4H), 3.84 (s, 6H). ¹³C-NMR (CD₃CN, 101 MHz): δ=162.0, 159.97, 159.92, 159.87, 151.8, 151.7, 151.3, 147.5, 138.0, 137.2, 130.1, 129.3, 129.0, 125.4, 125.2, 122.2, 121.4, 115.4. ESI-HRMS (pos. detection mode): calcd for C₄₈H₄₀N₆O₂Os m/z [M]²⁺ 462.1414; found: 462.1408. Elemental analysis calcd for C₄₈H₄₀F₁₂N₆O₂OsP₂+H₂O (%): C, 46.83, H, 3.44, N, 6.83; found: C, 46.92, H, 3.29, N, 6.34.

### [Ru(bpy)₂((E,E′)-4,4′-Bis[p-aminostyryl]-2,2′-bipyridine)][PF₆]₂ (17)

Compound 17 was synthesized following a procedure described in the literature (Storrier, G. D. and Colbran, S. B., 1997). Elemental analysis calcd for C₄₆H₃₈F₁₂N₈P₂Ru+3H₂O (%): C, 48.13, H, 3.86, N, 9.76; found: C, 48.09, H, 3.37, N, 10.05.

### [Ru(bpy)₂(4,4′-(E,E′)-2-(4-methoxyphenyl)prop-1-en-1-yl)-2,2′-bipyridine)][PF₆]₂ (18)

Under nitrogen, Ru(bpy)₂Cl₂ (63 mg, 0.13 mmol, 1.0 equiv.) and 4,4′-bis((E)-2-(4-methoxyphenyl)prop-1-en-1-yl)-2,2′-bipyridine (65 mg, 0.15 mmol, 1.1 equiv.) were suspended in dry ethanol and the mixture was refluxed for 15 h. Then the solution was cooled down and a sat. aqueous solution of NH₄PF₆ was added. The crude product, which precipitated as a PF₆ salt was collected by filtration and washed with water and diethyl ether. The product was purified by silica gel chromatography using acetonitrile/potassium nitrate 0.3 M in water (9:1 v/v) as eluent. The fractions containing the product were collected and the solvent evaporated. The residue was dissolved in acetonitrile and filtered. The filtrate was concentrated to dryness under vacuum. The residue was dissolved in ethanol and a sat. aqueous solution of NH₄PF₆ was added. The precipitate was filtered, washed with water and diethyl ether, and dried under vacuum to yield 18 (124 mg, 0.11 mmol, 85%) as a dark red solid. ¹H-NMR (CD₃CN, 400 MHz): δ=8.55-8.41 (m, 6H), 8.07 (tdd, J=7.8, 4.7, 1.5 Hz, 4H), 7.91-7.82 (m, 2H), 7.79-7.73 (m, 2H), 7.66-7.53 (m, 6H), 7.50-7.35 (m, 6H), 7.06-6.95 (m, 4H), 6.95-6.87 (m, 2H), 3.94-3.74 (m, 6H), 2.41-2.24 (m, 6H). ¹³C NMR (101 MHz, CD₃CN) δ=161.10, 160.60, 157.94, 157.69, 152.66, 152.60, 151.83, 148.61, 145.49, 138.61, 135.58, 129.86, 128.45, 127.64, 125.14, 124.79, 122.58, 114.86, 56.00, 18.11. ESI-HRMS (pos. detection mode): calcd for C₅₀H₄₄O₂N₆Ru m/z [M]²⁺ 431.1285; found: 431.1282. Elemental analysis calcd for C₅₀H₄₄F₁₂N₆O₂P₂Ru+2H₂O (%): C, 50.55, H, 4.07, N, 7.07; found: C, 50.73, H, 3.79, N, 7.21.

### [Ru(bpz)₂((E,E′)-4,4′-Bis[p-methoxystyryl]-2,2′-bipyridine)][PF₆]₂ (19)

Under nitrogen, Ru(bpz)₂Cl₂ (100 mg, 0.21 mmol, 1.0 equiv.) and (E,E′)-4,4′-Bis[p-methoxystyryl]-2,2′-bipyridine (104 mg, 0.25 mmol, 1.2 equiv.) were suspended in dry ethylene glycol (5 mL) and the mixture was heated at 130° C. for 24 h. Then the solution was cooled down and a sat. aqueous solution of NH₄PF₆ was added. The crude product, which precipitated as a PF₆ salt was collected by filtration and washed with water and diethyl ether. The product was purified by silica gel chromatography using acetonitrile/potassium nitrate 0.3 M in water (9:1 v/v) as eluent. The fractions containing the product were collected and the solvent evaporated. The residue was dissolved in acetonitrile and filtered. The filtrate was concentrated to dryness under vacuum. The residue was dissolved in ethanol and a sat. aqueous solution of NH₄PF₆ was added. The precipitate was filtered, washed with water and diethyl ether, and dried under vacuum to yield 19 (43 mg, 0.04 mmol, 19%) as a dark red solid. ¹H-NMR (CD₃CN, 400 MHz): δ=9.76 (t, J=1.5 Hz, 4H), 8.71 (d, J=1.9 Hz, 2H), 8.61 (dd, J=9.8, 3.2 Hz, 4H), 7.90 (dd, J=3.3, 1.2 Hz, 2H), 7.87 (dd, J=3.2, 1.2 Hz, 2H), 7.76 (d, J=16.4 Hz, 2H), 7.66 (d, J=8.9 Hz, 4H), 7.54 (d, J=6.0 Hz, 2H), 7.48 (dd, J=6.1, 1.8 Hz, 2H), 7.19 (d, J=16.3 Hz, 2H), 7.03 (d, J=8.8 Hz, 4H), 3.85 (s, 6H). ¹³C-NMR (CD₃CN, 101 MHz): δ=162.2, 157.2, 152.6, 151.7, 149.7, 149.34, 149.30, 147.9, 147.3, 146.1, 138.0, 130.2, 129.2, 125.3, 122.2, 121.6, 115.5, 56.1. ESI-HRMS (pos. detection mode): calcd for C₄₄H₃₆N₁₀O₂Ru m/z [M]²⁺ 419.1033; found: 419.1030. Elemental analysis calcd for C₄₄H₃₆F₁₂N₁₀O₂P₂Ru+4H₂O (%): C, 44.04, H, 3.70, N, 11.67; found: C, 44.11, H, 2.95, N, 11.60.

### [Ru(4,7-Diphenyl-1,10-phenanthroline)₃][PF₆]₂([Ru(bphen)₃][PF₆]2) (Comparative Example)

[Ru(bphen)₃][PF₆]₂ was synthesized as previously published (Crosby, G. A. and Watts, R. J., 1971) using RuCl₂dmso₄ precursor. Purity of the sample was assessed by HPLC and elemental analysis. Elemental analysis calcd for C₇₂H₄₈F₁₂N₆P₂Ru (%): C, 62.30, H, 3.49, N, 6.05; found: C, 62.28, H, 3.44, N, 5.92.

2. Photophysical Properties

Photophysical measurements were performed to evaluate the potential of the complexes as photosensitizers.

Spectroscopic Measurements

The absorption of the samples was measured with a SpectraMax M2 Spectrometer (Molecular Devices). The emission was measured by irradiation of the sample in fluorescence quartz cuvettes (width 1 cm) using a NT342B Nd-YAG pumped optical parametric oscillator (Ekspla) at 355 nm. Luminescence was focused and collected at right angle to the excitation pathway and directed to a Princeton Instruments Acton SP-2300i monochromator. As a detector, a XPI-Max 4 CCD camera (Princeton Instruments) has been used.

Luminescence Quantum Yield Measurements

For the determination of the luminescence quantum yield, the samples were prepared in an CH₃CN solution with an absorbance of 0.1 at 355 nm. This solution was irradiated in fluorescence quartz cuvettes (width 1 cm) using a NT342B Nd-YAG pumped optical parametric oscillator (Ekspla) at 355 nm. The emission signal was focused and collected at right angle to the excitation pathway and directed to a Princeton Instruments Acton SP-2300i monochromator. As a detector a XPI-Max 4 CCD camera (Princeton Instruments) has been used. The luminescence quantum yields were determined by comparison with the reference [Ru(bipy)₃]Cl₂ in CH₃CN (Φₑₘ=5.9%) (Nakamaru, K., 1982) applying the following formula:

Φ_{em, sample}=Φ_{em, reference}*(F_{reference}/Fₛₐₘₚₗₑ)*(Iₛₐₘₚₗₑ/I_{reference})*(nₛₐₘₚₗₑ/n_{reference})²

### F=1-10^{-A}

Φₑₘ=luminescence quantum yield, F=fraction of light absorbed, I=integrated emission intensities, n=refractive index, A=absorbance of the sample at irradiation wavelength.

Lifetime Measurements

For the determination of the lifetimes, the samples were prepared in an air saturated and in a degassed CH₃CN solution with an absorbance of 0.2 at 355 nm. This solution was irradiated in fluorescence quartz cuvettes (width 1 cm) using a NT342B Nd-YAG pumped optical parametric oscillator (Ekspla) at 355 nm. The emission signal was focused and collected at right angle to the excitation pathway and directed to a Princeton Instruments Acton SP-2300i monochromator. As a detector a R928 photomultiplier tube (Hamamatsu) has been used.

TABLE 1
Spectroscopic properties of characterised compounds 1-19
in CH₃CN at room temperature.

| | | | | τ/ns | |
|---|---|---|---|---|---|
| | λ_{abs}/nm (ε/M⁻¹ cm⁻¹ * 10⁻³) | λₑₘ/nm | ϕₑₘ | air | degassed |
| 1 | 300 (81.6), 385 (111.6), 515 | 677 | 0.019 | 86 | 385 |
| | (47.8) | | | | |
| 2 | 305 (109.5), 425 (133.4), 495 | 709 | <0.001 | 48 | 222 |
| | (111.8) | | | | |
| 3 | 305 (96.6), 370 (152.3), 495 | 682 | 0.011 | 76 | 338 |
| | (63.9) | | | | |
| 4 | 290 (57.0), 425 (93.5), 485 | 703 | 0.004 | 69 | 417 |
| | (82.6) | | | | |
| 5 | 295 (76.9), 360 (99.8), 475 | 674 | 0.014 | 36 | 231 |
| | (39.5) | | | | |
| 6 | 290 (79.0), 415 (57.3), 460 | 697 | 0.005 | 54 | 405 |
| | (61.4) | | | | |
| 7 | 290 (95.7), 365 (64.8), 465 | 664 | 0.028 | 96 | 542 |
| | (34.4) | | | | |
| 8 | 265 (99.6), 305 (30.7), 420 | 698 | 0.004 | 136 | 334 |
| | (57.0), 460 (57.9) | | | | |
| 9 | 265 (101.4), 290 (50.2), 360 | 663 | 0.019 | 193 | 669 |
| | (61.9), 465 (36.6) | | | | |
| 10 | 280 (125.9), 305 (56.6), 425 | 698 | 0.006 | 72 | 339 |
| | (64.2), 475 (70.4) | | | | |
| 11 | 280 (147.7), 305 (81.4), 360 | 663 | 0.027 | 108 | 679 |
| | (69.8), 475 (55.1) | | | | |
| 12 | 290 (71.5), 330 (37.8), 400 | — | — | — | — |
| | (13.3), 460 (20.5), 480 (18.2), | | | | |
| | 550 (1.5), 600 (0.2) | | | | |
| 13 | 290 (85.9), 330 (34.4), 360 | — | — | — | — |
| | (43.5), 410 (18.4), 460 (27.2), | | | | |
| | 480 (23.9), 550 (1.9), 600 | | | | |
| | (0.1) | | | | |
| 14 | 290 (87.3), 330 (33.1), 410 | — | — | — | — |
| | (16.7), 460 (26.1), 480 (23.2), | | | | |
| | 550 (1.9), 600 (0.2) | | | | |
| 15 | 290 (66.2), 360 (44.8), 420 | — | — | — | — |
| | (17.9), 460 (24.5), 480 (21.9), | | | | |
| | 550 (1.9), 600 (0.2) | | | | |
| 16 | 290 (60.1), 320 (31.2), 350 | — | — | — | — |
| | (38.6), 420 (18.3), 450 (21.5), | | | | |
| | 480 (19.0), 550 (7.4), 600 | | | | |
| | (5.1), 690 (3.5), 720 (1.6) | | | | |
| 17 | 290 (48.9), 330 (13.5), 390 | — | — | — | — |
| | (25.8), 440 (21.9), 470 (22.2), | | | | |
| | 480 (21.2), 550 (2.5), 600 | | | | |
| | (0.3) | | | | |
| 18 | 290 (64.6), 320 (24.6), 340 | — | — | — | — |
| | (28.4), 410 (9.6), 460 (18.4), | | | | |
| | 480 (14.2), 550 (1.0), 600 | | | | |
| | (0.06) | | | | |
| 19 | 300 (87.2), 330 (59.4), 350 | — | — | — | — |
| | (69.6), 410 (37.1), 430 (41.2), | | | | |
| | 480 (19.2), 550 (2.1), 600 | | | | |
| | (0.5) | | | | |

λ_{abs} = Absorption maximum, λₑₘ = Emission maximum, ϕₑₘ = Luminenscene Quantum Yield, τ = Lifetime.

Results

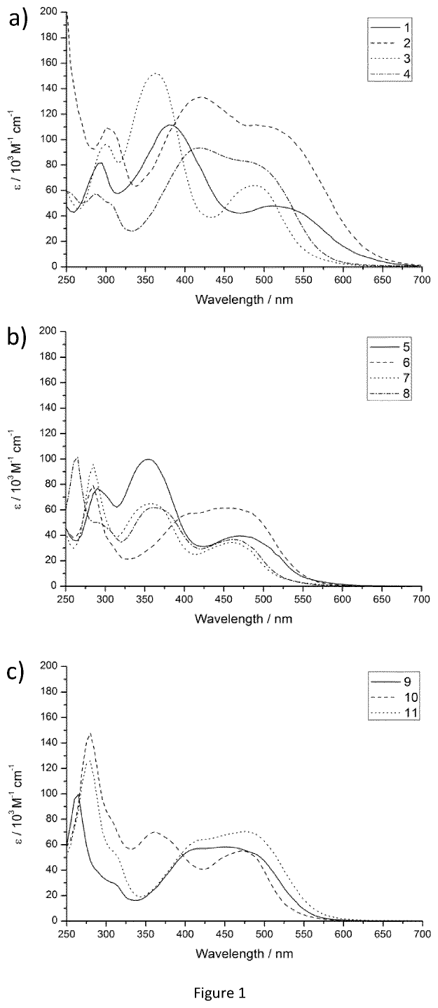

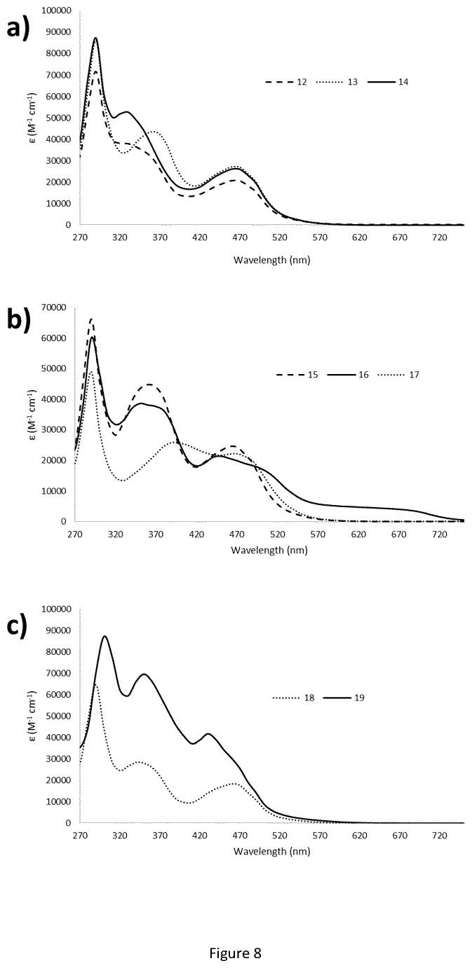

The photophysical properties of the prepared complexes were systematically investigated to evaluate their potential as PSs (Table 1). One crucial parameter in a PDT treatment is the penetration depth of the light and therefore the used wavelength. Based on this, the one-photon absorption spectra of the compounds were determined in CH₃CN (FIGS. 1 and 8). The compounds have generally a strong absorbance with a large red shift of about 50-70 nm for the symmetric Ru(II) complexes 1-3 in comparison to the prototype complex [Ru(bpy)₃]²⁺ with an MLCT band at 450 nm (Balzani, V. et al., 2007). The comparison between the symmetric 2-3 and asymmetric compounds 4-19 shows that the characteristic absorption profile stays the same while the red shift in absorption as well as the extinction coefficients are increasing with the number of (E,E′)-4,4′-Bisstyryl-2,2′-bipyridine ligands coordinated to the Ru(II) core. Importantly, the compounds have an absorption tail in the therapeutical window (600-900 nm) potentially enabling them for the treatment of deep seated tumors. More particularly, compound 16 absorbs light at up to 720 nm, which is much higher than most metal based PS.

Afterwards, the emission in CH₃CN has been determined for compounds 1-11 upon excitation at 355 nm. The emission of the compounds was measurable between 550-900 nm with a maximum between 664-709 nm. Worthy of note, the complexes which show a high red shift of the MLCT transition, have accordingly also the emission maximum at higher wavelengths. This large Stokes shift for all investigated compounds implies minimal inference between excitation and emission. Following this, the luminescence quantum yields were measured upon excitation at 355 nm. The comparison between the values shows luminescence quantum yield between 0.028-<0.001.

Consequently, the luminescence lifetimes were determined in degassed and air saturated CH₃CN upon excitation at 355 nm to investigate the influence of oxygen on the excited state. The measured lifetimes of the compounds 1-11 were found to be in the nanosecond range in a degassed solution between 222-542 ns and in an aerated solution between 36-96 ns. Importantly, for all Ru(II) polypyridyl complexes a decrease of the lifetime could be observed in an aerated solution in comparison to a degassed solution. This indicates that the excited state of the complex (³MLCT) is able to interact with a component in the air.

3. Singlet Oxygen Generation

The ability to generate reactive oxygen species upon light exposure was investigated

for compounds 1-11.

Electron Spin Resonance (ESR) Measurements

For verification of the reactive species formed upon light exposure of the compounds, the respective ESR spectra were recorded. The samples with a final concentration of 10 μM were dissolved in CH₃CN or PBS containing 20 mM TEMP (2,2,6,6-tetramethylpiperidine) as a ¹O₂ scavenger or 20 mM DMPO (5,5-dimethyl-1-pyrroline N-oxide) as a *OH radical scavenger. Capillary tubes were filled with the solution and sintered by fire. EPR spectra were recorded on a Bruker A300 spectrometer with 1 G field modulation, 100 G scan range and 20 mW microwave power. The samples were measured in exclusion from light and after irradiation for 60 s (450±10 nm, 21.8 mW cm⁻²).

Singlet Oxygen Measurements

Direct Evaluation

The samples were prepared in an air saturated CH₃CN or D₂O solution with an absorbance of 0.2 at 450 nm. This solution was irradiated in fluorescence quartz cuvettes (width 1 cm) using a mounted M450LP1 LED (Thorlabs) whose irradiation, centered at 450 nm, has been focused with aspheric condenser lenses. The intensity of the irradiation has been varied using a T-Cube LED Driver (Thorlabs) and measured with an optical power and energy meter. The emission signal was focused and collected at right angle to the excitation pathway and directed to a Princeton Instruments Acton SP-2300i monochromator. A longpass glass filter was placed in front of the monochromator entrance slit to cut off light at wavelengths shorter than 850 nm. As a detector an EO-817L IR-sensitive liquid nitrogen cooled germanium diode detector (North Coast Scientific Corp.) has been used. The singlet oxygen luminesce at 1270 nm was measured by recording spectra from 1100 to 1400 nm. For the data analysis, the singlet oxygen luminescence peaks at different irradiation intensities were integrated. The resulting areas were plotted against the percentage of the irradiation intensity and the slope of the linear regression calculated. The absorbance of the sample was corrected with an absorbance correction factor. As reference for the measurement Rose Bengal (Φ=76%) (Kochevar, I. E. and Redmond, R. W., 2000) was used and the singlet oxygen quantum yields were calculated using the following formula:

Φₛₐₘₚₗₑ=Φ_{reference}*(Sₛₐₘₚₗₑ/S_{reference})*(I_{reference}/Iₛₐₘₚₗₑ)

I=I₀*(1-10^{-A})

Φ=singlet oxygen quantum yield, S=slope of the linear regression of the plot of the areas of the singlet oxygen luminescence peaks against the irradiation intensity, I=absorbance correction factor, 10=light intensity of the irradiation source, A=absorbance of the sample at irradiation wavelength.

Indirect Evaluation

For the measurement in CH₃CN: The samples were prepared in an air-saturated CH₃CN solution containing the complex with an absorbance of 0.2 at the irradiation wavelength, N,N-dimethyl-4-nitrosoaniline aniline (RNO, 24 μM) and imidazole (12 mM). For the measurement in PBS buffer: The samples were prepared in an air-saturated PBS solution containing the complex with an absorbance of 0.1 at the irradiation wavelength, N,N-dimethyl-4-nitrosoaniline aniline (RNO, 20 μM) and histidine (10 mM). The samples were irradiated on 96 well plates with an Atlas Photonics LUMOS BIO irradiator for different times. The absorbance of the samples was measured during these time intervals with a SpectraMax M2 Microplate Reader (Molecular Devices). The difference in absorbance (A₀−A) at 420 nm for the CH₃CN solution or at 440 nm a PBS buffer solution was calculated and plotted against the irradiation times. From the plot the slope of the linear regression was calculated as well as the absorbance correction factor determined. The singlet oxygen quantum yields were calculated using the same formulas as used for the direct evaluation.

TABLE 2
Singlet oxygen quantum yields in CH₃CN and aqueous solution. Average
of three independent measurements. n.d. = not detectable.

| | direct | direct | indirect | indirect | indirect | indirect |
|---|---|---|---|---|---|---|
| | 450 nm | 450 nm | 450 nm | 450 nm | 540 nm | 540 nm |
| | CH₃CN | D₂O | CH₃CN | PBS | CH₃CN | PBS |
| 1 | 65% | n.d. | 66% | 5% | 61% | 3% |
| 2 | 18% | n.d. | 25% | 3% | 16% | 1% |
| 3 | 52% | n.d. | 48% | 6% | 49% | 5% |
| 4 | 34% | n.d. | 40% | 2% | 37% | 3% |
| 5 | 54% | n.d. | 51% | 8% | 46% | 6% |
| 6 | 46% | n.d. | 53% | 2% | 38% | 2% |
| 7 | 75% | n.d. | 77% | 11% | 68% | 10% |
| 8 | 59% | n.d. | 62% | 3% | 57% | 2% |
| 9 | 75% | n.d. | 82% | 9% | 79% | 8% |
| 10 | 51% | n.d. | 48% | 2% | 43% | 2% |
| 11 | 66% | n.d. | 73% | 7% | 72% | 5% |
| [Ru(phen)₃]²⁺ | 25% | n.d. | 23% | 2% | 22% | 2% |
| (PF₆⁻)₂ (*) | | | | | | |
| [Ru(bphen)₃]²⁺ | 44% | n.d. | 47% | 4% | 45% | 3% |
| (PF₆⁻)₂ (*) | | | | | | |

(*) comparative examples

For identification of the type of ROS produced upon light exposure, electron spin resonance (ESR) spectroscopy in CH₃CN as well as in phosphate-buffered saline (PBS) was employed. As a singlet oxygen (¹O₂) scavenger 2,2,6,6-tetramethylpiperidine (TEMP) and as a .OOH or .OH radical scavenger 5,5-dimethyl-1-pyrroline N-oxide (DMPO) was used. While no signal for the formation of a .OOH or .OH radicals were detected, the formation of ¹O₂ in CH₃CN and PBS was confirmed by observation of the characteristic ¹O₂-induced triplet signal in the ESR spectrum. Following this, the amount of generated ¹O₂ was quantitatively determined by two methods, namely 1) direct measurement of the phosphorescence of ¹O₂ and 2) indirect measurement of the variation in absorbance of a 102 scavenger and monitoring its change in absorbance. The singlet oxygen quantum yields (Table 2) were found to be between 16-82% in CH₃CN and 1-11% in an aqueous solution. Therefore, the compounds of the invention 1-11 are able to produce 102 in an efficient way. Overall, compound 7 was found to have an impressive singlet oxygen production (i.e., CH₃CN: 68-77%, aqueous solution: 10-11%).

4. Stability

As a crucial parameter in view medicinal applications, the stability of the compounds in a biological environment was investigated as previous investigations have shown that this could be problematic for metal complexes.

Stability in Human Plasma

The stability of the complexes 1-11 was evaluated with caffeine as an internal standard, which has already been shown to be suitable for these experiments (Guy, P. A. et al., 2009). The pooled human plasma was obtained from Biowest and caffeine from TCI Chemicals. Stock Solutions of the compounds (20 μM) and caffeine (40 μM) were prepared in DMSO. One aliquot of the solutions was added to 975 μL of human plasma to a total volume of 1000 μL. Final concentrations of the compounds of 0.25 μM and caffeine of 0.5 μM were achieved. The resulting solution was incubated for 48 h at 37° C. with continuous gentle shaking (ca. 300 rpm). The reaction was stopped after the incubation time by addition of 3 mL of methanol. The mixture was centrifuged for 60 min at 3000 rpm at 4° C. The methanolic solution was filtered through a 0.2 μm membrane filter. The solvent was evaporated under reduced pressure and the residue was dissolved in 1:1 (v/v) CH₃CN/H₂O 0.1% TFA solution. The solution was filtered through a 0.2 μm membrane filter and analyzed using a HPLC System. The solvents (HPLC grade) were millipore water (0.1% TFA, solvent A) and acetonitrile (solvent B). Method M1: 0-3 minutes: isocratic 50% A (50% B); 3-17 minutes: linear gradient from 50% A (50% B) to 0% A (100% B); Method M2: 0-3 minutes: isocratic 95% A (5% B); 3-17 minutes: linear gradient from 95% A (5% B) to 0% A (100% B); 17-23 minutes: isocratic 0% A (100% B). The flow rate was 1 mL/min and the chromatogram was detected at 250 nm.

Photostability

The samples were prepared in an air saturated CH₃CN solution. To measure the photostability, the samples were irradiated at 450 nm in 96 well plates with an Atlas Photonics LUMOS BIO irradiator during time intervals from 0-10 min. The absorbance spectrum from 350-700 nm was recorded with a SpectraMax M2 Microplate Reader (Molecular Devices) after each time interval and compared. As a positive control [Ru(bipy)₃]Cl₂ and as a negative control Protoporphyrin IX has been used.

Results

To assess the compatibility of the here reported compounds under biological conditions, their stability in human pooled plasma was tested. For this purpose, the compounds were incubated in human plasma at 37° C. for 48 h and after this time extracted from the plasma. The compounds were analysed before and after giving them into the human plasma via HPLC. As an internal standard, caffeine has been used. The comparison of the HPLC chromatograms shows no change before and after the incubation for the compounds 1-11 which indicates the stability of the compounds under biological conditions. Next to the stability in human plasma, the stability upon irradiation was investigated. Importantly, currently approved photosensitizers are associated with a low stability upon irradiation. For this purpose, the compounds were exposed to irradiation at 450 nm and their UV/Vis absorption monitored in constant intervals. [Ru(bipy)₃]Cl₂ has been used as a positive control and Protoporphyrin IX has been used as a negative control. The results show that the photobleaching effect is correlating with the coordinated ligand. The complexes 1-11 show little to no photobleaching in comparison to the photosensitizer Protoporphyrin IX, which completely changed in the investigated time interval.

5. Dark and (Photo)-Cytotoxicity in Monolayer Cells

The compounds 1-19 were tested in various cell lines to determine their ability to act as a photosensitizer.

Cell Culture

The human glioblastoma astrocytoma (U373) cell line was cultured in MEM medium supplemented with 10% FBS, 1% NEAA (non-essential amino-acids) and 1% penicillin/streptomycin. The human cervical carcinoma (HeLa), doxorubicin-resistant human colon adenocarcinoma (SW620/AD300) and the mouse colon carcinoma (CT-26) cell lines were cultured in DMEM medium supplemented with 10% FBS and 1% penicillin/streptomycin. The human retinal epithelial cells (RPE-1, non-cancerous cells immortalized with hTERT) were cultured in DMEM-F12 medium supplemented with 10% FBS and 1% penicillin/streptomycin. All cell lines were obtained from the American Type Culture Collection (ATCC) and cultured at 37° C. and 5% CO₂. Before an experiment, the cells were passaged three times.

(Photo-)Cytotoxicity on 2D Cell Monolayers

The cytotoxicity of the compounds was assessed by measuring cell viability using a fluorometric resazurin assay. The cultivated cells were seeded in triplicates in 96 well plates with a density of 4000 cells per well in 100 μL of media. After 24 h, the medium was removed and the cells were treated with increasing concentrations of the compound diluted in cell media achieving a total volume of 200 μL. The cells were incubated with the compound for 4 h. After this time, the media was removed and replaced with 200 μL of fresh medium. For the phototoxicity studies, the cells were exposed to light with an Atlas Photonics LUMOS BIO irradiator. Each well was constantly illuminated with either a 480 nm or 540 nm irradiation. During this time, the temperature was maintained constantly at 37° C. The cells were grown in the incubator for additional 44 h. For the determination of the dark cytotoxicity, the cells were not irradiated and after the medium exchange directly incubated for 44 h. After this time, the medium was replaced with fresh medium containing resazurin with a final concentration of 0.2 mg/mL. After 4 h incubation, the amount of the fluorescent product resorufin was determined upon excitation at 540 nm and measurement its emission at 590 nm using a SpectraMax M2 Microplate Reader (Molecular Devices).

The obtained data was analyzed with the GraphPad Prism software.

TABLE 3
IC₅₀ values in μM in the dark and upon irradiation at 480 nm for compounds 1-18 in comparison to [Ru(phen)₃]²⁺ (PF₆⁻)₂,
[Ru(bphen)₃]²⁺ (PF₆⁻)₂, cisplatin and Protoporphyrin IX (PpIX) in non-cancerous retinal pigment epithelium (RPE-1) and nm for
compounds 1-19 in comparison to [Ru(phen)₃]²⁺ (PF₆⁻)₂, [Ru(bphen)₃]²⁺ (PF₆⁻)₂, cisplatin and Protoporphyrin IX (PpIX) in
human cervical carcinoma (HeLa) cells. Compounds 1-11 were also tested at 540 nm. Average of three independent measurements.

| | RPE-1 | | | | | HeLa | | | | |
|---|---|---|---|---|---|---|---|---|---|---|
| | | 480 nm | | 540 nm | | | 480 nm | | 540 nm | |
| | | (10 min, | | (40 min, | | | (10 min, | | (40 min, | |
| | dark | 3.1 J/cm²) | PI | 9.5 J/cm²) | PI | dark | 3.1 J/cm²) | PI | 9.5 J/cm²) | PI |
| 1^{a)} | >100 | 30.9 ± 0.8 | >3.2 | 31.7 ± 1.2 | >3.2 | >100 | 29.5 ± 1.2 | >3.4 | 48.7 ± 4.5 | >2.1 |
| 2^{a)} | >100 | 38.4 ± 8.1 | >2.6 | 49.0 ± 6.2 | >2.0 | >100 | 29.4 ± 8.3 | >3.4 | 52.3 ± 6.1 | >1.9 |
| 3^{a)} | >100 | 53.6 ± 3.2 | >1.9 | 44.9 ± 2.9 | >2.2 | >100 | 15.3 ± 1.4 | >6.5 | 11.3 ± 2.2 | >8.8 |
| 4 | >100 | 10.9 ± 2.7 | >9.2 | 14.7 ± 3.1 | >6.8 | >100 | 8.5 ± 1.2 | >11.8 | 10.6 ± 2.2 | >9.4 |
| 5 | >100 | 10.2 ± 2.1 | >9.8 | 12.7 ± 2.9 | >7.9 | >100 | 6.4 ± 1.7 | >16.7 | 7.9 ± 1.3 | >12.7 |
| 6 | >100 | 7.3 ± 1.2 | >13.7 | 8.1 ± 1.6 | >12.3 | >100 | 5.3 ± 0.6 | >18.9 | 5.8 ± 0.9 | >17.2 |
| 7 | >100 | 3.1 ± 0.8 | >32.3 | 8.4 ± 1.3 | >11.9 | >100 | 1.2 ± 0.4 | >83.3 | 1.5 ± 0.5 | >66.7 |
| 8 | >100 | 9.2 ± 1.6 | >10.9 | 15.6 ± 2.3 | >6.4 | >100 | 12.6 ± 1.3 | >7.9 | 11.4 ± 1.5 | >8.8 |
| 9 | >100 | 2.4 ± 0.9 | >41.7 | 2.1 ± 0.7 | >47.6 | >100 | 1.5 ± 0.6 | >66.7 | 1.2 ± 0.7 | >83.3 |
| 10 | 15.6 ± 1.3 | 1.8 ± 0.3 | 8.7 | 2.1 ± 0.4 | 7.4 | 10.7 ± 1.4 | 1.6 ± 0.3 | 6.7 | 1.7 ± 0.4 | 6.3 |
| 11 | 20.8 ± 1.5 | 0.9 ± 0.4 | 23.1 | 0.7 ± 0.3 | 29.7 | 16.5 ± 0.8 | 0.6 ± 0.3 | 27.5 | 0.7 ± 0.5 | 23.6 |
| 12 | >100 | 1.6 ± 0.2 | >63 | — | — | >100 | 0.6 ± 0.3 | >167 | — | — |
| 13 | >100 | 1.52 ± 0.09 | >66 | — | — | 62 ± 10 | 0.5 ± 0.1 | >124 | — | — |
| 14 | >100 | 1.47 ± 0.06 | >68 | — | — | >100 | 0.8 ± 0.1 | >125 | — | — |
| 15 | >100 | 11 ± 2 | >9 | — | — | >100 | 1.0 ± 0.3 | >100 | — | — |
| 16 | >100 | 30 ± 7 | >3 | — | — | >100 | 27 ± 6 | >4 | — | — |
| 17 | >100 | 8 ± 1 | >13 | — | — | >100 | 1.4 ± 0.1 | >71 | — | — |
| 18 | >100 | 33 ± 4 | >3 | — | — | >100 | 16.7 ± 0.7 | >6 | — | — |
| 19 | — | — | — | — | — | >100 | 59 ± 8 | >2 | — | — |
| [Ru(phen)₃]²⁺ | >100 | >100 | n.d. | >100 | n.d. | >100 | >100 | n.d. | >100 | n.d. |
| (PF₆⁻)₂ | | | | | | | | | | |
| [Ru(bphen)₃]²⁺ | 12.1 ± 0.6 | 0.9 ± 0.2 | 13.4 | 1.2 ± 0.3 | 10.1 | 8.5 ± 0.4 | 0.7 ± 0.2 | 12.1 | 1.1 ± 0.2 | 7.7 |
| (PF₆⁻)₂ | | | | | | | | | | |
| PpIX | >100 | 3.8 ± 0.1 | >26.3 | 3.3 ± 0.1 | >30.3 | >100 | 2.5 ± 0.1 | >40.0 | 2.1 ± 0.3 | >47.6 |
| cisplatin | 29.3 ± 1.4 | — | — | — | — | 10.5 ± 0.8 | — | — | — | — |

^{a)}due to solubility limitations the compounds were investigated as chloride salts.

TABLE 4
IC₅₀ values in μM in the dark and upon irradiation at 480 and 540 nm for compounds 1-11 in comparison to
[Ru(phen)₃]²⁺ (PF₆⁻)₂, [Ru(bphen)₃]²⁺ (PF₆⁻)₂, cisplatin and Protoporphyrin IX (PpIX) in mouse colon carcinoma
(CT-26) and human glioblastoma astrocytoma (U373) cells. Average of three independent measurements.

| | CT-26 | | | | | U373 | | | | |
|---|---|---|---|---|---|---|---|---|---|---|
| | | 480 nm | | 540 nm | | | 480 nm | | 540 nm | |
| | | (10 min, | | (40 min, | | | (10 min, | | (40 min, | |
| | dark | 3.1 J/cm²) | PI | 9.5 J/cm²) | PI | dark | 3.1 J/cm²) | PI | 9.5 J/cm²) | PI |
| 1^{a)} | >100 | 20.3 ± 1.8 | >4.9 | 33.4 ± 3.5 | >3.0 | >100 | 51.7 ± 3.2 | >1.9 | 83.1 ± 6.7 | >1.2 |
| 2^{a)} | >100 | 42.6 ± 3.8 | >2.3 | 62.5 ± 8.2 | >1.6 | >100 | 49.1 ± 6.0 | >2.0 | 61.4 ± 5.6 | >1.6 |
| 3^{a)} | >100 | 19.3 ± 2.1 | >5.2 | 23.0 ± 2.6 | >4.3 | >100 | 41.0 ± 3.5 | >2.4 | 51.3 ± 3.4 | >1.9 |
| 4 | >100 | 7.3 ± 1.4 | >13.7 | 9.6 ± 1.9 | >10.4 | >100 | 14.3 ± 2.4 | >7.0 | 19.0 ± 2.8 | >5.3 |
| 5 | >100 | 5.1 ± 1.1 | >19.6 | 6.2 ± 1.0 | >16.1 | >100 | 15.3 ± 2.2 | >6.5 | 17.8 ± 3.2 | >5.6 |
| 6 | >100 | 2.4 ± 0.5 | >41.7 | 3.1 ± 0.3 | >32.3 | >100 | 8.3 ± 1.1 | >12.0 | 10.7 ± 1.9 | >9.3 |
| 7 | >100 | 0.7 ± 0.4 | >142.9 | 0.9 ± 0.3 | >111.1 | >100 | 10.5 ± 1.7 | >9.5 | 13.5 ± 1.6 | >7.4 |
| 8 | >100 | 8.2 ± 1.1 | >12.2 | 7.8 ± 0.9 | >12.8 | >100 | 11.7 ± 1.5 | >8.5 | 13.3 ± 2.4 | >7.5 |
| 9 | >100 | 1.1 ± 0.5 | >90.9 | 0.9 ± 0.4 | >111.1 | >100 | 2.5 ± 0.9 | >40.0 | 2.1 ± 0.7 | >47.6 |
| 10 | 5.2 ± 0.8 | 1.3 ± 0.3 | 4.0 | 1.1 ± 0.4 | 4.7 | 13.7 ± 1.1 | 1.8 ± 0.3 | 7.6 | 2.1 ± 0.4 | 6.5 |
| 11 | 8.6 ± 0.9 | 0.5 ± 0.2 | 17.2 | 0.6 ± 0.2 | 14.3 | 18.5 ± 1.0 | 0.9 ± 0.2 | 20.6 | 0.7 ± 0.3 | 26.4 |
| [Ru(phen)₃]²⁺ | >100 | >100 | n.d. | >100 | n.d. | >100 | >100 | n.d. | >100 | n.d. |
| [Ru(bphen)₃]²⁺ | 2.8 ± 0.5 | 0.4 ± 0.2 | 7.0 | 0.5 ± 0.2 | 5.6 | 10.1 ± 0.7 | 1.3 ± 0.2 | 7.8 | 1.2 ± 0.3 | 8.4 |
| PpIX | 2.8 ± 0.5 | 0.4 ± 0.2 | 7.0 | 0.5 ± 0.2 | 5.6 | 10.1 ± 0.7 | 1.3 ± 0.2 | 7.8 | 1.2 ± 0.3 | 8.4 |
| cisplatin | 6.5 ± 1.1 | — | — | — | — | 17.6 ± 1.7 | — | — | — | — |

^{b)} due to solubility limitations the compounds were investigated as chloride salts.

Results

To investigate the toxicity of the compounds, the IC₅₀ values in the dark as well as upon light exposure at 480 (10 min, 3.1 i/cm²) and 540 nm (40 min, 9.5 i/cm²) were determined. For this as a control of non-cancerous cells retinal pigment epithelium (RPE-1) was chosen and as cancerous cells human cervical carcinoma (HeLa), mouse colon carcinoma (CT-26) and human glioblastoma astrocytoma (U373) were chosen. Importantly for a PS, in all investigated cell lines (Table 3 and 4) the compounds 1-9 and 12-19 were found to be non-toxic in the dark (IC_{50, dark}>100 IM, IC_{50, dark}=62 l-M for compound 13), and the bphen coordinated compounds (10-11) showed little dark cytotoxicity which does not prevent their use as PS. The results show that all compounds are able to act as a PS with PI values from >1.2->167. Compounds 7, 9 and 12-15 display a phototoxicity on HeLa cells in the low micromolar range while being non-toxic in the dark. More particularly, the impressive ability of compounds 7 and 12 with PI values of >142.9 and >167 in CT-26 and HeLa cells respectively could have been unveiled.

6. Dark and (Photo)-Cytotoxicity in 3D Multicellular Tumour Spheroids

The abilities of compounds 1-11 were investigated in 3D multicellular tumour spheroids (MCTS) as this presents a model which is closer to clinically treated tumours.

Generation and Analysis of 3D Multicellular Tumor Spheroids (MCTS) A suspension of 0.75% agarose in PBS buffer was heated inside a high-pressure

autoclave. The hot emulsion was transferred into wells (50 μL per well) of a 96 cell culture well plate. The plates were exposed for 3 h to UV irradiation to ensure the sterility and allow the agarose solution to cool down. After this time, the agarose was overlayed with a HeLa cell suspension at a density of 3000 cells per well in 150 μL of media. The MCTS were cultivated and maintained at 37° C. in a cell culture incubator at 37° C. with 5% CO₂ atmosphere. The culture media was replaced every two days. Within two-three days MCTs were formed from the cell suspension. The formation as well as integrity, diameter and volume of the MCTs was monitored by an Axio Observer Z₁ (Carl Zeiss) phase contrast microscope. The volume was calculated using the following formula: V=4/3πr³. The luminescence images along the z-axis were captured by a one-(λₑₓ=458 nm, λₑₘ=600-750 nm) or two-photon (λₑₓ=800 nm, λₑₘ=600-750 nm) excitation in the z-stack mode with a an LSM 880 (Carl Zeiss) laser scanning confocal microscope equipped with Argon and a Coherent Chameleon 2-Photon laser and a GaAsP detector.

Results

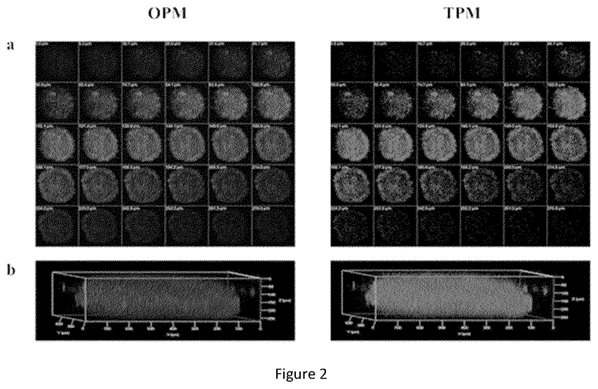

After analysis of the effect that the compounds of the invention have on monolayer cells, their ability in 3D multicellular tumour spheroids (MCTS) has been investigated for compounds 1-11. MCTS are an employed tissue model for the assessment of the delivery of drugs as it is closer to clinically treated tumours. Worthy of note, many investigated anticancer agents have failed at the transition of a cancer monolayer cell to in vivo studies. Partly this is been attributed to the failed drug delivery through the penetration of extracellular barriers. It has been shown that small MCTS with diameters of 200 μm are able to simulate intercellular interactions and therefore investigate the drug delivery. Recent studies have shown that the investigation of larger MCTS can also mimics the pathological conditions found in solid tumours as hypoxia in the tumour centre and its proliferation gradients. Consequently, we have chosen to investigate MCTS with diameters of 600-800 μm as an in vivo model. For this purpose, HeLa MCTS were incubated with the compounds 1-11 and the distribution of the compounds analysed via one and two-photon Z-stack imaging microscopy. After incubation for 12 h shows that the compounds 6-11 show a strong luminescence signal at every section depth corresponding with a complete penetration of the compounds in the MCTS. This is illustrated on FIG. 2 for compound 7. Similar results have been obtained for the others tested compounds.

3D Multicellular Tumor Spheroids (MCTS) Growth Inhibition Assay

MCTS were treated with the corresponding compounds (20 μM 1-7, 20 μM tetraphenylporphyrin H₂TPP, 10 μM cisplatin, 30 μM cisplatin, 2% DMSO, v %) by replacing 50% of the media with drug supplemented media in the dark for three days. After this time, the MCTS were exposed to a two-photon irradiation (800 nm, 10 J/cm²) with a section interval of 5 μm using a LSM 880 (Carl Zeiss) laser scanning confocal microscope equipped with a Coherent Chameleon 2-Photon laser. The cell culture media was replaced every two days. The integrity and diameter of the MCTs was monitored with an Axio Observer Z₁ (Carl Zeiss) phase contrast microscope every 24 h.

Results

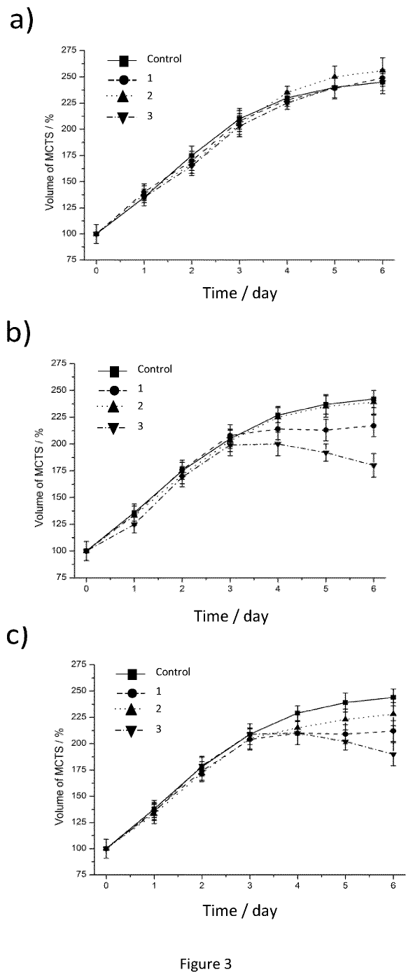

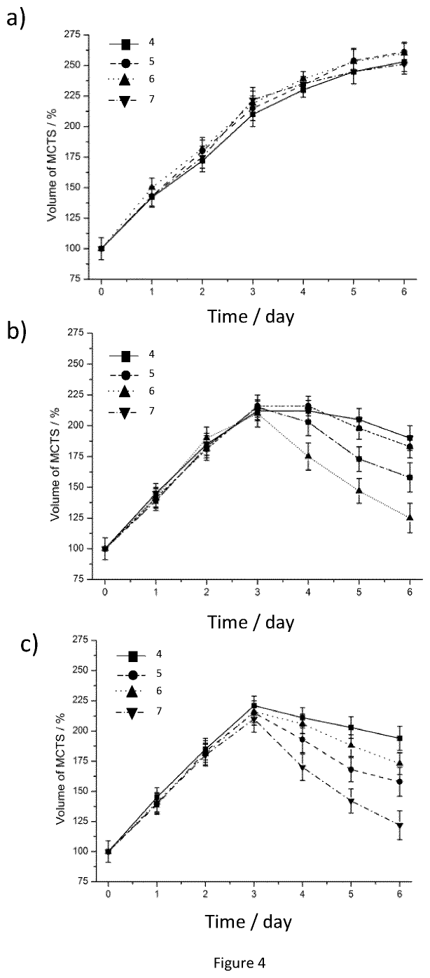

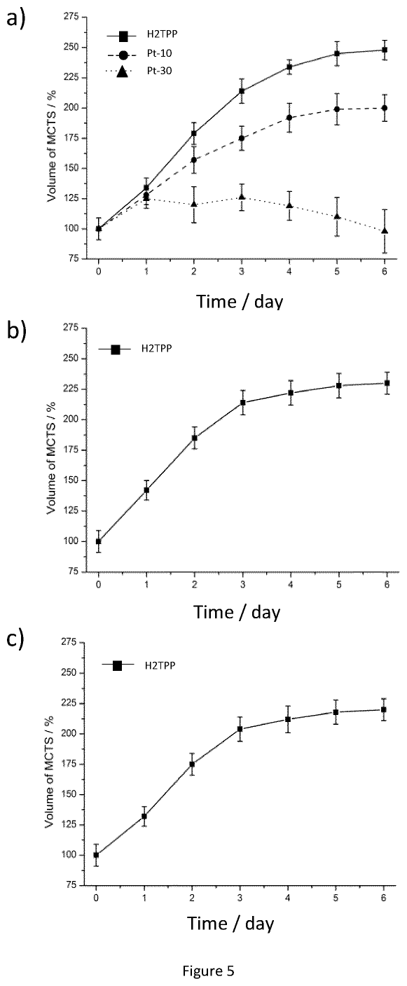

MCTS with a diameter of about 800 μm were incubated with compounds 1-7 (20 μM, 2% DMSO, v %), tetraphenylporphyrin (H₂TPP) (20 μM, 2% DMSO, v %), cisplatin (10 μM and 30 μM) for three days strictly in the dark. The MCTS were then exposed to 1P irradiation (500 nm, 10 J/cm²) or 2P irradiation (800 nm, 10 J/cm², section interval of 5 μm) on day 3. During the whole time period, the shape and volume of the MCTS was constantly monitored. As expected, the volume of the MCTS in the control group which was treated purely with cell media/DMSO as well as of compounds 1-7 and H₂TPP (FIGS. 3, 4 and 5) were increasing in a similar manner, indicating that the investigated compounds do not show any inhibitory effect in the dark. Contrary to this, cisplatin showed a weak effect on the tumours growth at 10 μM, whereas it was significantly decreasing the volume of the MCTS at 30 μM. In comparison, the volume of the MCTS treated with compounds 1-7 and exposed to light irradiation significantly decreased, demonstrating their strong tumour inhibition effect. As expected from previous investigations on cell monolayers presented above, compound 7 had the strongest phototoxic effect. Under identical conditions, the treatment with H₂TPP had only slight effect, demonstrating the ability of compound 7 to act as a PDT PS at low drug doses and low light doses.

3D Multicellular Tumor Spheroids (MCTS) Viability Assay

MCTS were treated with the corresponding compounds (20 μM 1-7, 2% DMSO, v %) by replacing 50% of the media with drug supplemented media in the dark for three days. After this time, the MCTS were exposed to a two-photon irradiation (800 nm, 10 J/cm²) with a section interval of 5 μm using a LSM 880 (Carl Zeiss) laser scanning confocal microscope equipped with a Coherent Chameleon 2-Photon laser. The cell culture media was replaced every two days. Two days after the irradiation the MCTS viability was tested using a Viability/Cytotoxicity Kit for mammalian cells (Invitrogen). Living cells can be identified from dead cells through the presence of ubiquitous intracellular esterase activity which can be monitored by the enzymatic conversion of the non-fluorescent calcein AM to the intensely fluorescent calcein (λₑₓ=495 nm, λₑₘ=515 nm). As the spectroscopic properties of the dead cell probe EthD-1 overlaps with the one of the investigated compounds, this probe was not used and only calcein AM as a probe for living cells was used. MCTS were incubated with calcein AM (2 μM) for 30 min and images of the MCTS taken with an Axio Observer Z1 (Carl Zeiss, Germany) inverted fluorescence microscope.

Results

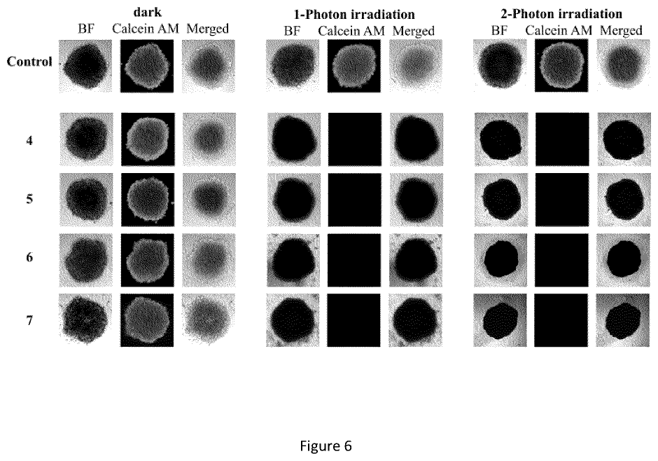

To further study the effect the complexes on tumour survival, the treated MCTS were stained with calcein AM (FIG. 6), which can identify living from dead cells through the presence of ubiquitous intracellular esterase activity, converting the non-fluorescent calcein AM into the intensely fluorescent calcein. As expected from the tumour growth inhibition assay, the MCTS treated with compounds 4-7 in the dark showed a strong green fluorescence signal, indicating that the MCTS are still intact. Contrary to this, light treatment by 1P irradiation (500 nm, 10 J/cm²) or 2P irradiation (800 nm, 10 J/cm², section interval of 5 μm) had a drastic effect on cell survival in the MCTS. No significant fluorescence signal for compounds 4-7 could be observed, indicating that the MCTS were mostly eradicated.

(Photo-)Cytotoxicity on 3D Multicellular Tumor Spheroids (MCTS)

The cytotoxicity of the compounds in 3D multicellular tumor spheroids (MCTS) was assessed by measurement of the ATP concentration. MCTS were treated with increasing concentrations of the compound (2% DMSO, v %) by replacing 50% of the media with drug supplemented media and incubation for 12 h. After this time, the MCTS were divided in three identical groups. The first group was strictly kept in the dark. The second group was exposed to a one-photon irradiation (500 nm, 10 J/cm²) using a LED and the third group was exposed to a two-photon irradiation (800 nm, 10 J/cm²) with a section interval of 5 μm using a LSM 880 (Carl Zeiss, Germany) laser scanning confocal microscope equipped with a Coherent Chameleon 2-Photon laser. After the irradiation, all groups were incubated additional 48 h. The ATP concertation was measured using a CellTiter-Glo 3D Cell Viability kit (Promega) by measuring the generated chemiluminescence with an infinite M200 PRO (Tecan) plate reader. The obtained data was analyzed with the GraphPad Prism software.

TABLE 5
IC₅₀ in μM values in the dark and upon 1-Photon irradiation
(500 nm, 10 J/cm²) irradiation or 2-Photon irradiation (800 nm, 10
J/cm², section interval of 5 μm) for compounds 1-11 in comparison
to cisplatin and tetraphenylporphyrin (H₂TPP) in HeLa MCTS.
Average of three independent measurements.

| | | 1-Photon | | 2-Photon | |
|---|---|---|---|---|---|
| | | irradiation | | irradiation | |
| | | (500 nm, | | (800 nm, | |
| | dark | 10 J/cm²) | PI | 10 J/cm²) | PI |
| 4 | >100 | 78.3 ± 5.1 | >1.3 | 63.0 ± 4.2 | >1.6 |
| 5 | >100 | 19.3 ± 2.7 | >5.2 | 13.4 ± 3.9 | >7.5 |
| 6 | >300 | 33.8 ± 3.4 | >8.9 | 26.5 ± 2.9 | >11.3 |
| 7 | >300 | 6.8 ± 0.2 | >44.1 | 1.4 ± 0.2 | >214.3 |
| 8 | >300 | 32.6 ± 2.5 | >9.2 | 27.8 ± 3.1 | >10.8 |
| 9 | >300 | 7.5 ± 0.2 | >40.0 | 1.2 ± 0.3 | >250.0 |
| 10 | 27.8 ± 3.6 | 8.9 ± 0.7 | 3.1 | 3.1 ± 0.6 | 9.0 |
| 11 | 29.3 ± 2.9 | 3.8 ± 0.4 | 7.7 | 0.8 ± 0.5 | 36.6 |
| H₂TPP | >100 | >100 | n.d. | >100 | >100 |
| cisplatin | 18.6 ± 1.3 | — | — | — | — |

^{a)} due to solubility limitations the compounds were investigated as chloride salts.

Results

To quantify the photodynamic effect that the compounds have on HeLa MCTS, the (photo-)cytotoxicity (Table 5) was determined in the dark as well as upon 1 Photon (500 nm, 10 J/cm²) and 2 Photons irradiation (800 nm, 10 J/cm², section interval of 5 μm) by measurement of the ATP concentration of living cells through conversion into chemiluminescence. Importantly, no measurable cytotoxicity in the dark could be observed in HeLa MCTS for all compounds. The compounds 4-6, 8, 10 were found to be phototoxic in the micromolar range with PI values from >1.1 to >11.3. In comparison, compounds 7, 9 and 11 were found with much higher phototoxicity. These results confirm the (photo-)cytotoxicity evaluation on 2D monolayer cells. As a promising compound, 7 was identified. Importantly, no significant toxicity in the dark (IC_{50, dark}>300 μM) and high phototoxicity in the low micromolar range (IC_{50, 500} nm>6.8±0.2 μM, IC_{50, 800 nm}>1.4±0.2) with a PI value of >44.1 or >214.3, respectively were determined.

These results are very promising in comparison to H₂TPP which did not have a cytotoxic effect (IC_{50, dark}=IC_{50, 500 nm}=IC_{50, 800 nm}>100 μM) under identical experimental conditions. The comparison between 1 Photon and 2 Photons irradiation demonstrates a stronger 2 Photons phototoxicity, which is likely attributed to the deeper penetration depth of the longer wavelength as the intensity of the light is declining with tissue penetration. Overall, the highly phototoxic compound 7 in MCTS, which is able to act efficiently upon 1 Photon and 2 Photon irradiation could be unveiled.

7. In Vivo Evaluation

Based on the remarkable properties of compound 7, this compound was investigated inside a mouse model.

In Vivo Experiment

6 weeks age nu/nu female mice were purchased from Charles River. Compound 7 was dissolved into physiological saline at first. Tumor xenograft: 6×10⁶ SW620/AD300 cells were subcutaneously (s.c.) injected in the nude mice, the cells were suspension in 150 μL Matrigel (Corning) and saline (1:1, v/v). After a week, the tumor volumes of the mice reached approximately 80 mm³.

In Vivo (Photo-)Cytotoxicity

30 SW620/AD300 nude tumour-bearing nude mice were randomly separated into 6 groups and five mice for each group.

Group 1: injected 7 (2 mg/Kg 50 μL) intravenously and irradiate under 800 nm laser (50 mW, 1 kHz, pulse width 35 fs, 5 s/mm) 1 h after injection;

Group 2: injected 7 (2 mg/Kg 50 μL) intravenously and irradiate under 500 nm light (10 mW/cm², 60 min);

Group 3: injected by physiological saline (50 μL) and treated with 800 nm laser (50 mW, 1 kHz, pulse width 35 fs, 5 s/mm) 1 h after injection;

Group 4: injected by physiological saline (50 μL) and treated with 500 nm light (10 mW/cm², 60 min);

Group 5: intravenous injected 7 (2 mg/Kg 50 μL);

Group 6: injected 50 μL physiological saline.

The mice were anesthetized by the injection of 4% chloral hydrate aqueous solution (0.2 mL/20 g) before treatment. The tumour volume and body weight were measured and record for each two days. Tumor volume was calculated by the following formula

$$Volume = \frac{Length \times {Width}^{2}}{2}$$

Histological Examination

After the treatment, the mice were euthanized. The tumour was collected and fixated by 4% paraformaldehyde, and then obtained as paraffin-embedded samples and stained with hematoxylin and eosin (H&E). A Carl Zeiss Axio Imager Z2 microscope was used to observe the tissue structure and cell state of the sections.

Results

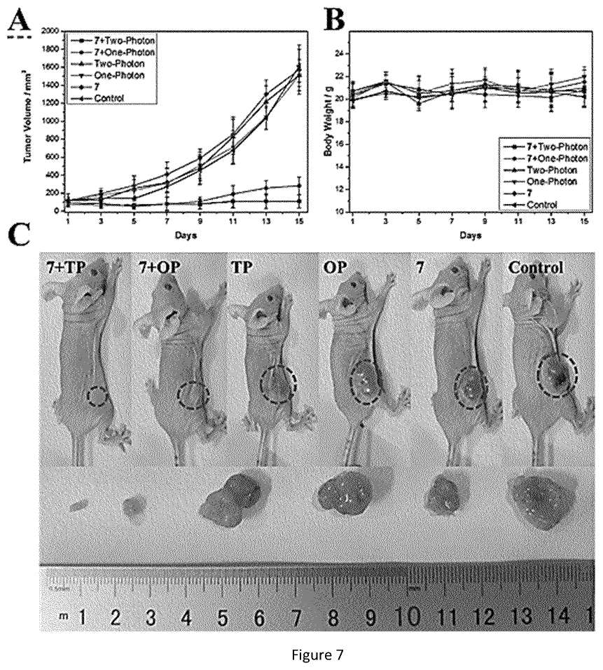

In vivo PDT experiments were prepared on SW620/AD300 (doxorubicin-selected P-gp-overexpressing human colon cancer cell) tumour-bearing nude mice. After the tumours volume reached 80 mm³, the mice were randomly separated into six groups and treated (day 1); group 1: injection of 2 mg/Kg 7 intravenously and 2P irradiation at 800 nm, group 2: injection of 2 mg/Kg 7 intravenously and 1P irradiation at 500 nm, group 3 and 4: were injection of physiological saline and treated with 1P and 2P irradiation, group 5: injection of 2 mg/Kg 7 intravenously and group 6 injection of the same volume of physiological saline. Importantly, the animals treated with 7 behave normally, without signs of pain, stress or discomfort. The body weight and tumour volume were recorded every two days (FIG. 7). Encouragingly, the PDT treated tumour drastically shrank until they were nearly eradicated whereas the tumours of the groups which were only treated by light or compound 7 did not show any significant effect. At day 15, all the nude mice were sacrificed and the tumour and organs were separated. The histological examination was demonstrated by H&E stain.

### REFERENCES

S. Monro, K. L. Colon, H. Yin, J. Roque, P. Konda, S. Gujar, R. P. Thummel, L. Lilge, C. G.
Cameron, S. A. McFarland, Chem. Rev. 2019, 119, 797-828.
Heinemann F. W., Karges J., Gasser G. Acc Chem Res 2017, 50, 2727-2736.
Agostinis P., Berg K., Cengel K. A., Foster T H, Girotti A. W., Gollnick S. O. et al. C A Cancer J Clin 2011, 61, 250-281.
Wachter E, Heidary D K, Howerton B S, Parkin S, Glazer E C. Chem Commun 2012, 48, 9649-9651.
Ogawa K, Kobuke Y. 2008, 8, 269-279.
Feuvrie, C., Maury, O., Le Bozec, H., Ledoux, I., Morrall, J. P., Dalton, G T., Samoc, M., Humphrey, M. G. J. Phys. Chem. A 2007, 111, 8980-8985.
Zuniga César et al., Polyhedron, Pergamon Press, Oxford, GB, vol. 85, 28 Sep. 2014 (2014 Sep. 28), pp 511-518
Francisco Gajardo et al. Inorganic Chemistry, vol. 50, no 13, 4 Jul. 2011, (2011 Jul. 4), pp 5910-5924
Paulina Dreyse et al. Dalton Transactions, vol. 42, no 43, 1 Jan. 2013 (2013 Jan. 1), page 15502
Ayman A. Abdel-Shafi et al. Journal of Physical Chemistry A. Molecules, Spectroscopy, Kinetics, Environment and General Theory, vol. 104 no 2, 1 Jan. 2000 (2000 Jan. 1), pp 192-202
C. E. McCusker and J. K. McCusker, Inorg. Chem., 2011, 50, 1656-1669.
Karges J., Basu U., Blacque O., Chao H., Gasser G., Angew. Chem. Int. Ed, 2019, accepted. DOI: 10.1002/anie.201907856.
Karges J., Blacque O., Chao H., Gasser G. Inorg Chem. 2019, accepted. DOI: 10.1021/acs.inorgchem.9b02019.
B. Sullivan, D. Salmon, T. Meyer, Inorg. Chem., 1978, 17, 3334-3341C.
Leidner, B. P. Sullivan, R. Reed, B. White, M. Crimmins, R. W. Murray and T. J. Meyer, Inorg. Chem., 1987, 26, 882-891.
K. Balasubramanian, V. Cammarata and Q. Wu, Langmuir, 1995, 11, 1658-1665.
Bratsos and E. Alessio, Inorg. Synth., 2010, 35, 148-152.
Zuloaga, F.; Kasha, M. Photochemistry and Photobiology, 1968, 7, 549-555.
G. A. Crosby and R. J. Watts, J. Am. Chem. Soc., 1971, 93, 3184-3188.
K. Nakamaru, Bull. Chem. Soc. Jpn., 1982, 55, 1639-1640.
S. Campagna, F. Puntoriero, F. Nastasi, G. Bergamini and V. Balzani, in Photochemistry and Photophysics of Coordination Compounds I, eds. V. Balzani and S. Campagna, Springer Berlin Heidelberg, Berlin, Heidelberg, 2007, DOI: 10.1007/128_2007_133, pp. 117-214.
I. E. Kochevar and R. W. Redmond, in Methods Enzymol., Academic Press 2000, vol. 319, pp. 20-28.
S. J. Bruce, I. Tavazzi, V. r. Parisod, S. Rezzi, S. Kochhar and P. A. Guy, Anal. Chem., 2009, 81, 3285-3296.
Aranyos, V. et al., J. Chem. Soc., Dalton Trans., 2001, 1319-1325.
Sinha, S. et al., RSC Adv., 2015, 5, 99529-99539.
Balasubramanian K. K. et al., Langmuir 1995, 11, 5, 1658-1665.
Woo, S. J. et al., ACS Catal. 2019, 9, 3, 2580-2593.
Storrier, G. D. and Colbran, S. B., Polyhedron, 16, 1997, Pages 2705-2710.

1-15. (canceled)

16. A method of treatment by photodynamic therapy comprising administering to an animal or a human in need thereof an effective amount of a compound of the following formula (I): 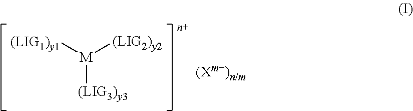
or a pharmaceutically acceptable salt and/or solvate thereof, as photosensitizer agent,
wherein
M is selected among ruthenium, rhenium, osmium, rhodium, iridium and platinum,
LIG₁ is a bidentate ligand having the following formula: 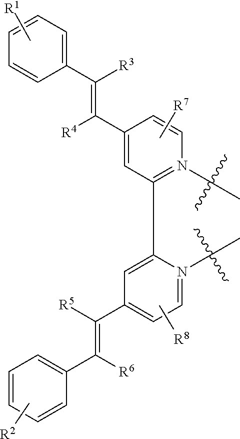
wherein
the wavy lines indicate the points of attachment to M,
R¹ and R² each independently represent one or several substituents selected in the group consisting of H, halogen, C₁-C₆ alkyl, C₆-C₁₀ aryl, OR⁹ and NR¹⁰R¹¹,
R³ to R⁶ each independently represent a substituent selected in the group consisting of H, halogen, C₁-C₆ alkyl, C₆-C₁₀ aryl, OR¹² and NR¹³R¹⁴,
R⁷ and R⁸ each independently represent one or several substituents selected in the group consisting of H, halogen, C₁-C₆ alkyl, C₆-C₁₀ aryl, OR¹⁵ and NR¹⁶R¹⁷,
R⁹ to R¹¹ are each independently selected in the group consisting of H and C₁-C₆ alkyl, and
R¹² to R¹⁷ are each independently selected in the group consisting of H, C₁-C₆ alkyl and CO—(C₁-C₆ alkyl),
LIG₂ is a bidentate ligand having the following formula (a) or (b): 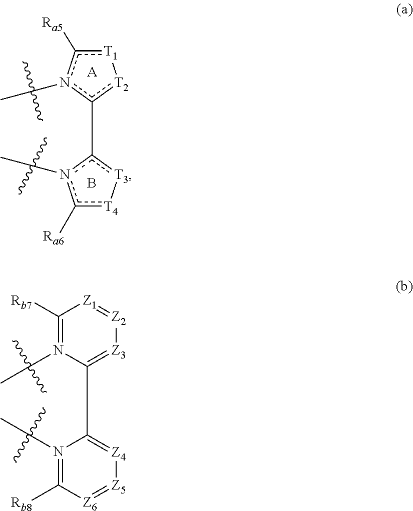
wherein the wavy lines indicate the points of attachment to M,
LIG₃ is a bidentate ligand having the following formula (c) or (d): 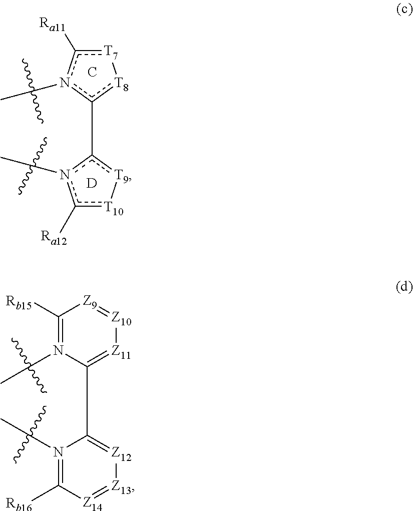
wherein the wavy lines indicate the points of attachment to M,
each [symbol] represents a single or a double bond, provided that each cycle A, B, C and D is a heteroaromatic cycle,
T₁ is NRₐ₁ or CRₐ₁, T₂ is NRₐ₂ or CRₐ₂, T₃ is NRₐ₃ or CRₐ₃, T₄ is NRₐ₄ or CRₐ₄, T₇ is NRₐ₇ or CRₐ₇, T₈ is NRₐ₈ or CRₐ₈, T₉ is NRₐ₉ or CRₐ₉ and T₁₀ is NRₐ₁₀ or CRₐ₁₀, provided that when T₁ is NRₐ₁, then T₂ is CRₐ₂, when T₃ is NRₐ₃, then T₄ is CRₐ₄, when T₇ is NRₐ₇, then T₈ is CRₐ₈ and when T₉ is NRₐ₉, then T₁₀ is CRₐ₁₀,
Z₁ is N or CR_{b1}, Z₂ is N or CR_{b2}, Z₃ is N or CR_{b3}, Z₄ is N or CR_{b4}, Z₅ is N or CR_{b5}, Z₆ is N or CR_{b6}, Z₉ is N or CR_{b9}, Z₁₀ is N or CR_{b10}, Z₁₁ is N or CR_{b11}, Z₁₂ is N or CR_{b12}, Z₁₃ is N or CR_{b13} and Z₁₄ is N or CR_{b14}, provided that at least two of Z₁ to Z₃ and at least two of Z₄ to Z₆ and at least two of Z₉ to Z₁₁ and at least two of Z₁₂ to Z₁₄ are not N,
Rₐ₁ to Rₐ₁₂ and R_{b1} to R_{b16} each independently represent H, halogen, optionally substituted C₁-C₆ alkyl, optionally substituted C₂-C₆ alkenyl, optionally substituted C₂-C₆ alkynyl, optionally substituted carbocycle, optionally substituted aryl, optionally substituted heteroaryl, optionally substituted heterocycle, CN, NO₂, N₃, COR¹⁸, OR¹⁹ or NR²⁰R²¹,
or Z₃ and Z₄ in formula (b) are linked together so that LIG₂ represents: 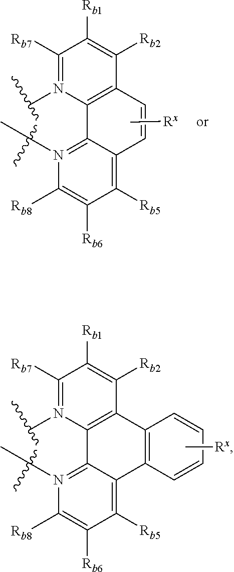
and/or
Z₁₁ and Z₁₂ are linked in formula (d) together so that LIG₃ represents: 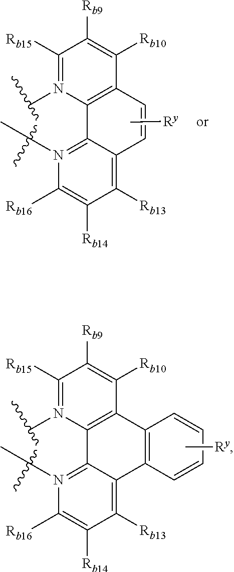
wherein R^{x} and R^{y} each independently represent one or several substituents selected in the group consisting of H, halogen, optionally substituted C₁-C₆ alkyl, optionally substituted C₂-C₆ alkenyl, optionally substituted C₂-C₆ alkynyl, optionally substituted carbocycle, optionally substituted aryl, optionally substituted heteroaryl, optionally substituted heterocycle, CN, NO₂, N₃, COR¹⁸, OR¹⁹ and NR²⁰R²¹,
R¹⁸ is selected in the group consisting of H, optionally substituted C₁-C₆ alkyl, OR²² and NR²³R²⁴,
R¹⁹ to R²⁴ are each independently selected in the group consisting of H, optionally substituted C₁-C₆ alkyl and optionally substituted CO—(C₁-C₆ alkyl),
X^{m-} is a pharmaceutically acceptable anion,
m and n are independently 1, 2, 3 or 4, wherein n is 1 when M is rhenium, n is 2 when M is ruthenium or osmium, n is 3 when M is rhodium or iridium and n is 4 when M is platinum, and
y1 is 1, 2 or 3, y2 and y3 are independently 0, 1 or 2, provided that y1+y2+y3 is 3.

17. The method according to claim 16, wherein R¹ and R² are one or several substituents each independently selected from the group consisting of H, halogen, OR⁹ and NR¹⁰R¹¹.

18. The method according to claim 17, wherein R¹ and R² are both OR⁹, with R⁹ being a C₁-C₆ alkyl.

19. The method according to claim 16, wherein LIG₁ is of following formula:

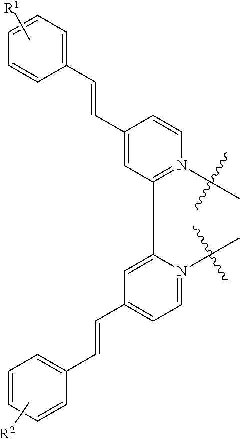

20. The method according to claim 16, wherein LIG₂ and LIG₃ are different from LIG₁.

21. The method according to claim 16, wherein Rₐ₁ to Rₐ₁₂ and R_{b1} to R_{b16} each independently represent H, halogen, C₁-C₆ alkyl, aryl, OR¹⁹ or NR²⁰R²¹, and
R^{x} and R^{y} each independently represent one or several substituents selected in the group consisting of H, halogen, C₁-C₆ alkyl, aryl, OR¹⁹ and NR²⁰R²¹,
with R¹⁹ to R²¹ being each independently selected in the group consisting of H and C₁-C₆ alkyl.

22. The method according to claim 16, wherein Rₐ₁ to Rₐ₁₂ and R_{b1} to R_{b16} are H or aryl, and R^{x} and R^{y} represent H.

23. The method according to claim 16, wherein y1 is 1, 2 or 3, y2 is 2, 1 or 0 respectively and y3 is 0.

24. The method according to claim 23, wherein LIG₂ is a bidentate ligand which is selected from the group consisting of: 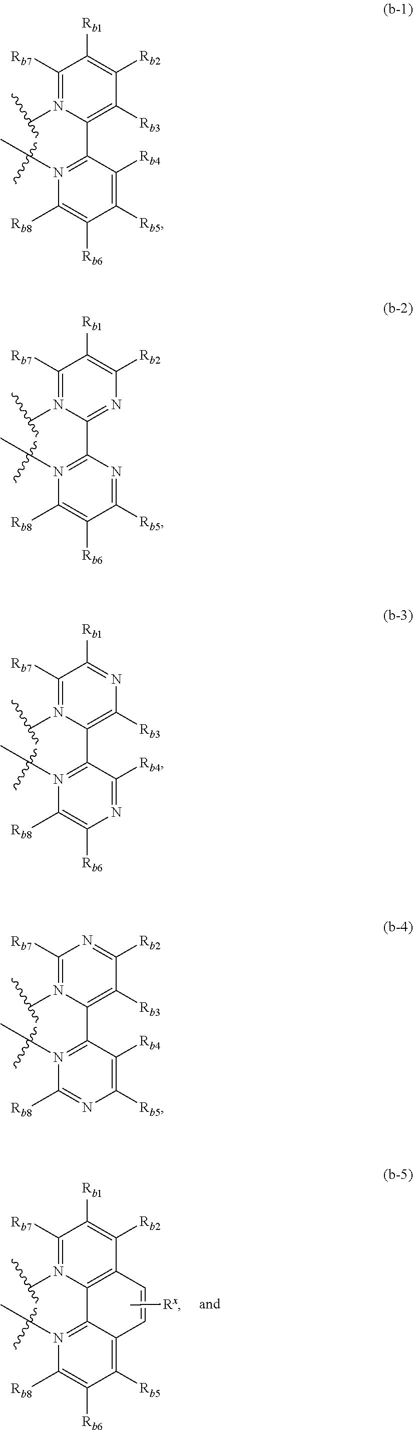 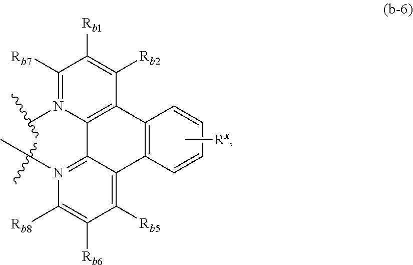
with R_{b1} to R_{b16} and R^{x} as defined in claim 16.

25. The method according to claim 24, wherein LIG₂ is of formula (b-1).

26. The method according to claim 16, wherein M is ruthenium or osmium.

27. The method according to claim 16, wherein the compound of formula (I) is selected from the group consisting of:

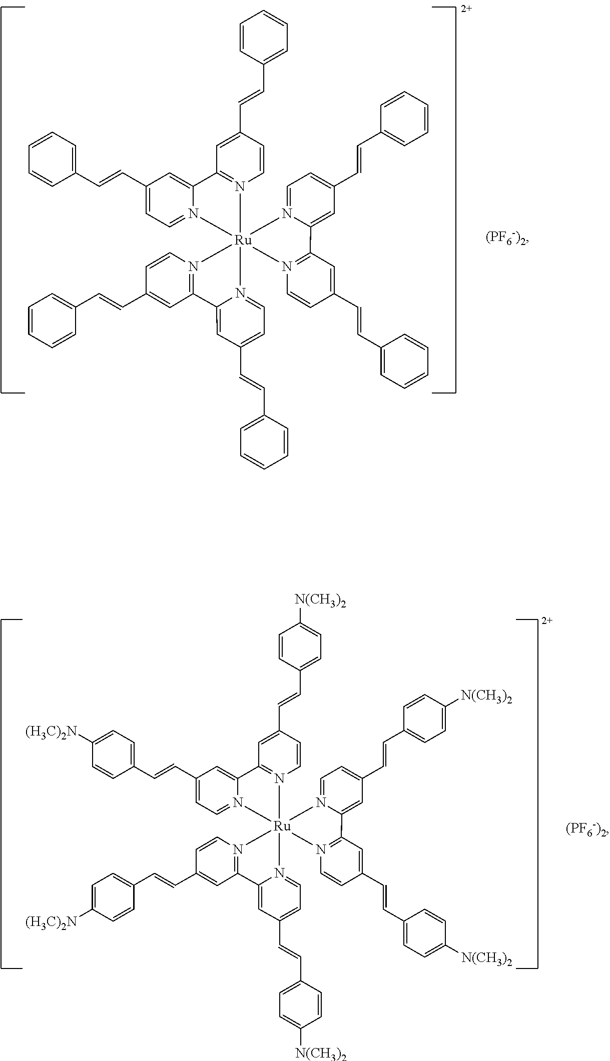

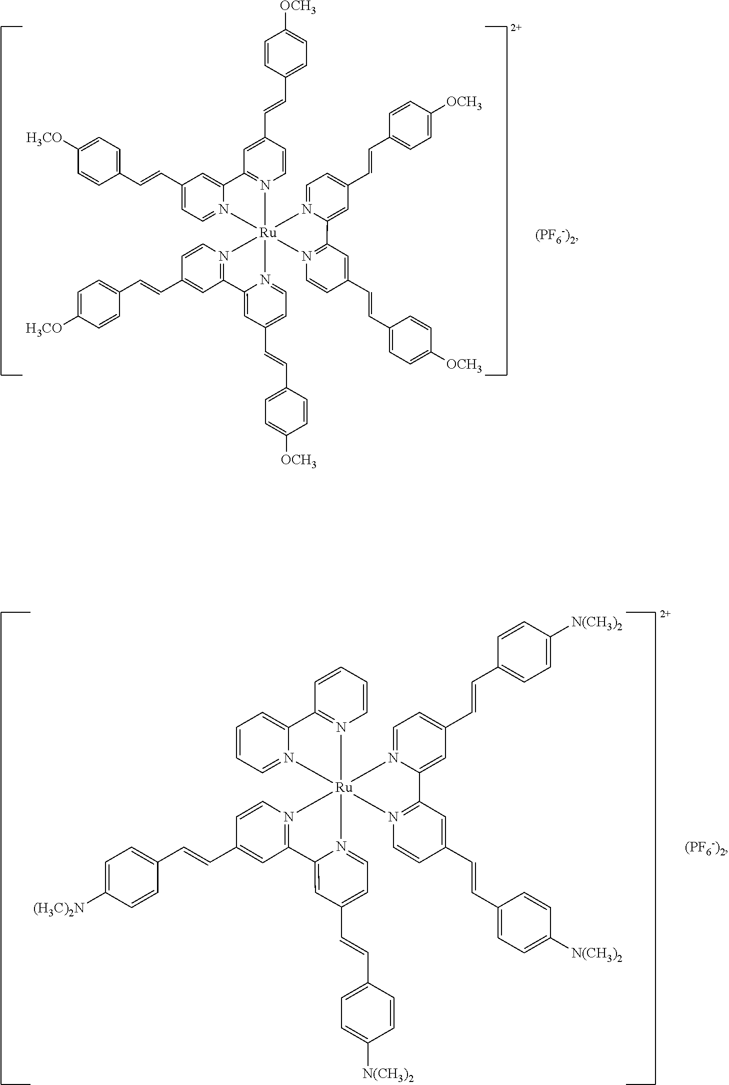

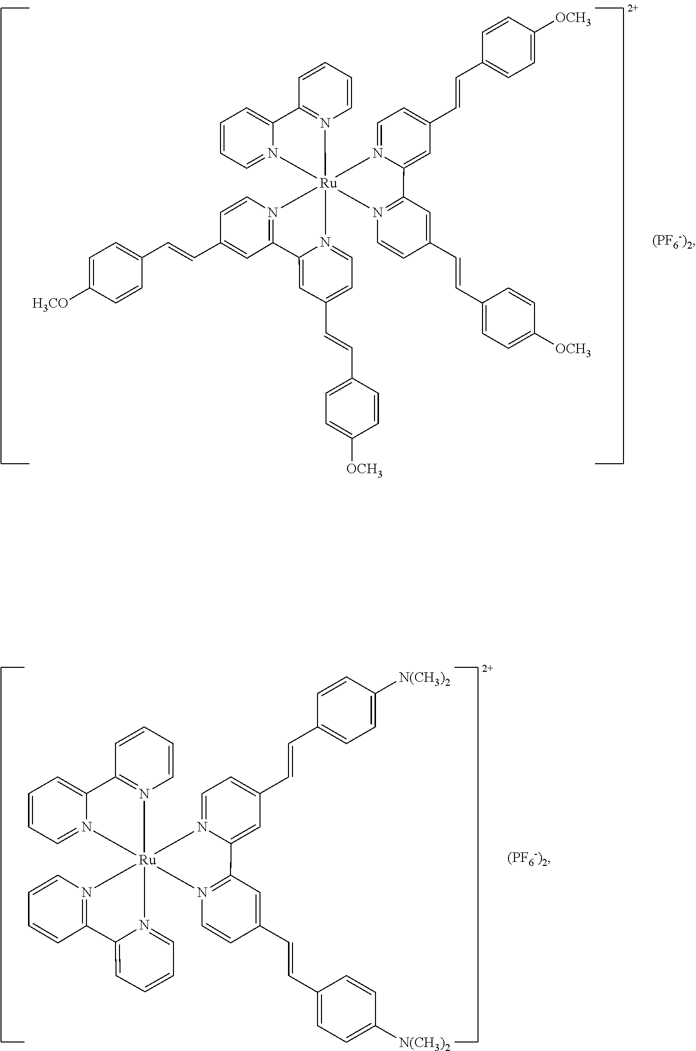

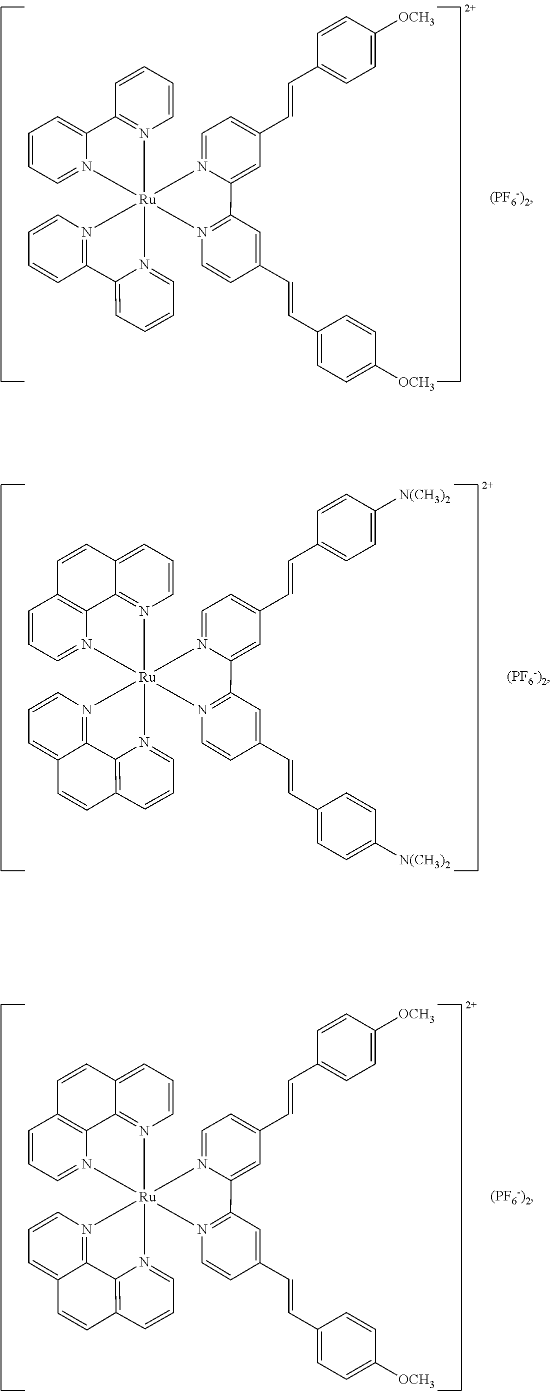

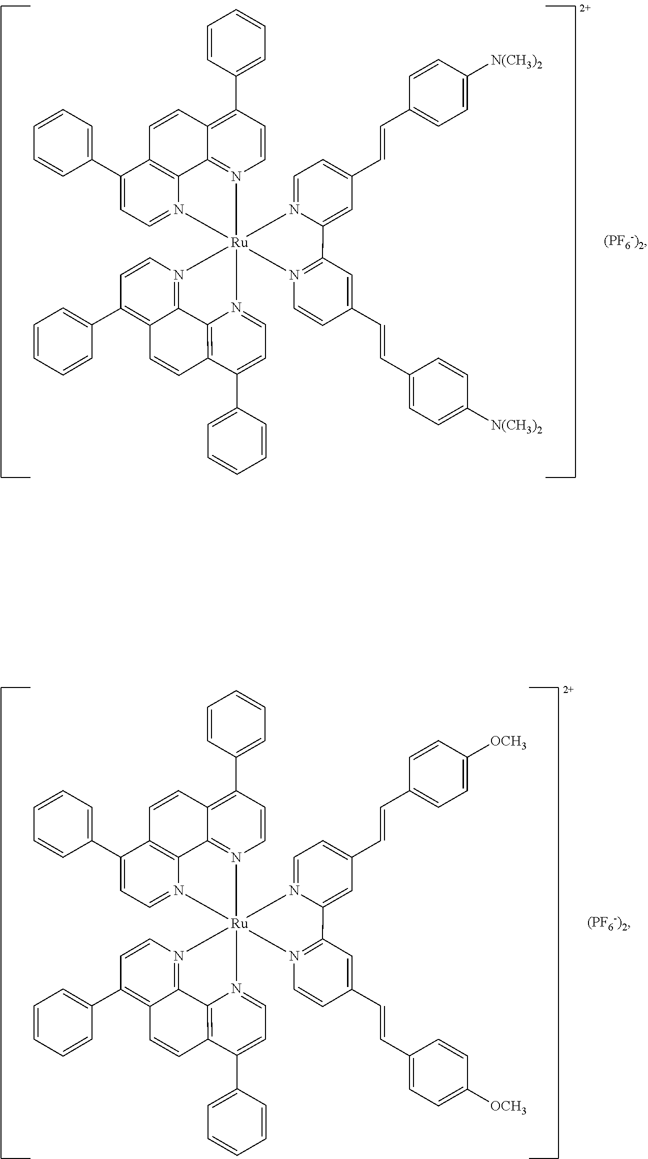

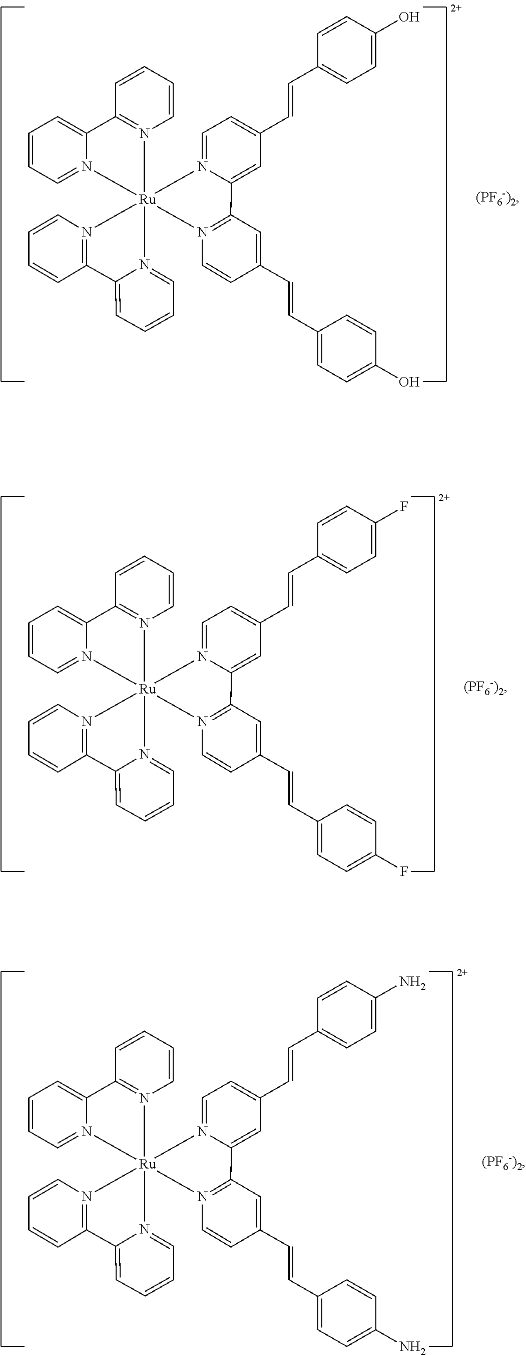

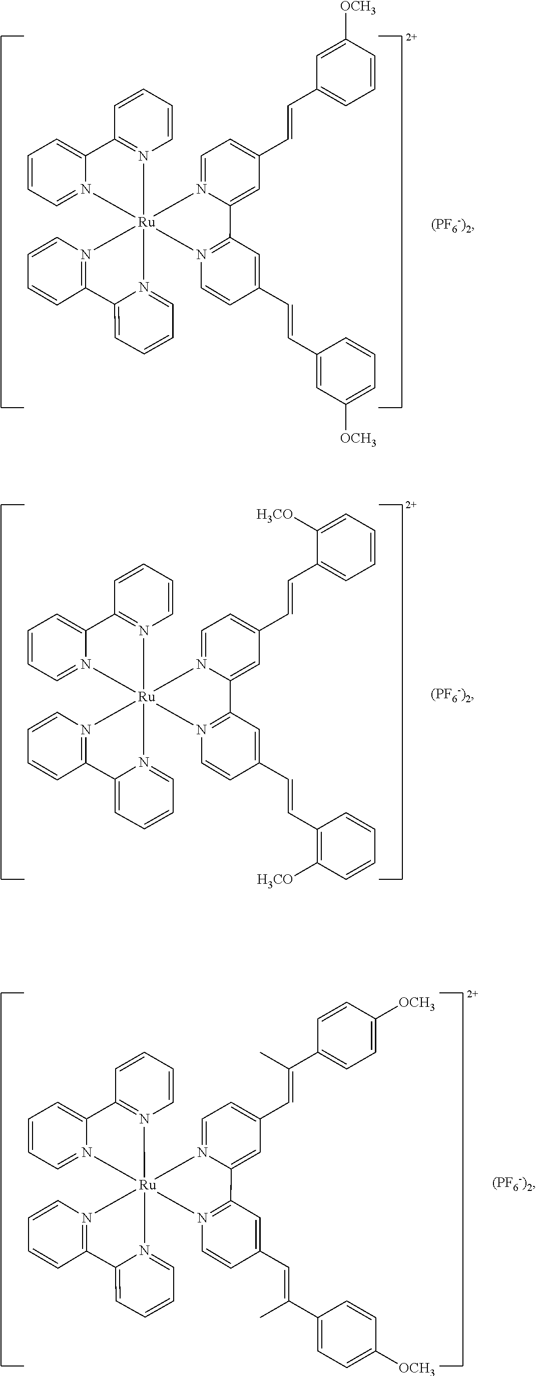

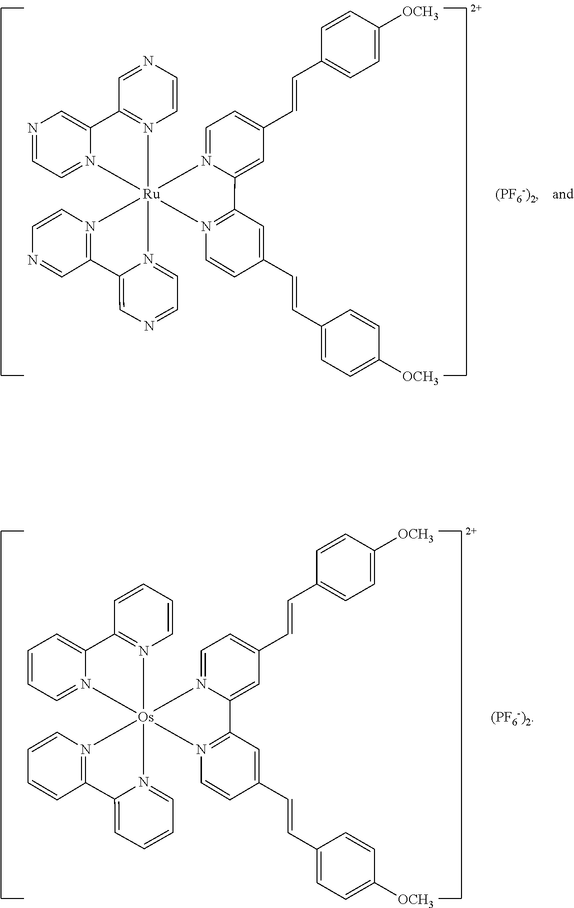

28. The method according to claim 16, wherein the photodynamic therapy is intended to treat a disease selected from the group consisting of cancer; bacterial infection; fungal infection; viral infection; and skin disorders.

29. The method according to claim 28, wherein the cancer is selected from the group consisting of lung cancer, bladder cancer, oesophageal cancer, colon cancer, stomach cancer, liver cancer, skin cancer, ovarian cancer, pancreatic cancer, head and neck cancer and brain cancer, the bacterial infection is selected from the group consisting of sinusitis, diabetic feet and burned wounds, the fungal infection is mycoses, the viral infections is herpes and the skin disorders are selected in the group consisting of acne and port wine stains.

30. A pharmaceutical composition comprising at least one compound of formula (I) as defined in claim 16 and at least one pharmaceutically acceptable excipient.

31. A method of treatment by photodynamic therapy comprising administering to an animal or a human in need thereof an effective amount of a pharmaceutical composition according to claim 30.

32. The method according to claim 31, wherein the photodynamic therapy is intended to treat a disease selected from cancer; bacterial infection; fungal infection; viral infection; and skin disorders.

33. A compound of formula (I): 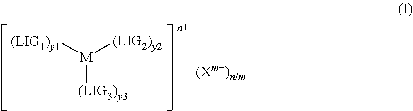
or a pharmaceutically acceptable salt and/or solvate thereof,
wherein
M is selected among ruthenium, rhenium, osmium, rhodium, iridium and platinum,
LIG₁ is a bidentate ligand having the following formula: 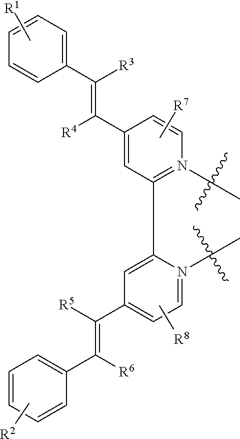
wherein
the wavy lines indicate the points of attachment to M,
R¹ and R² each independently represent one or several substituents selected in the group consisting of H, halogen, C₁-C₆ alkyl, C₆-C₁₀ aryl, OR⁹ and NR¹⁰R¹¹,
R³ to R⁶ each independently represent a substituent selected in the group consisting of H, halogen, C₁-C₆ alkyl, C₆-C₁₀ aryl, OR¹² and NR¹³R¹⁴,
R⁷ and R⁸ each independently represent one or several substituents selected in the group consisting of H, halogen, C₁-C₆ alkyl, C₆-C₁₀ aryl, OR¹⁵ and NR¹⁶R¹⁷,
R⁹ to R¹¹ are each independently selected in the group consisting of H and C₁-C₆ alkyl, and
R¹² to R¹⁷ are each independently selected in the group consisting of H, C₁-C₆ alkyl and CO—(C₁-C₆ alkyl),
LIG₂ is a bidentate ligand having the following formula (a) or (b): 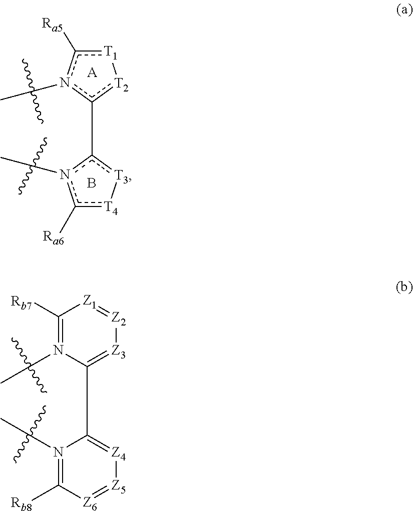
wherein the wavy lines indicate the points of attachment to M,
LIG₃ is a bidentate ligand having the following formula (c) or (d): 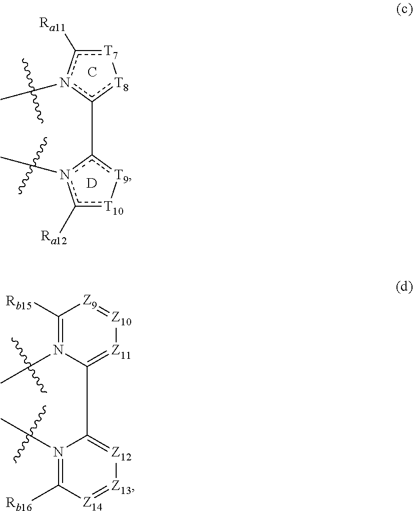
wherein the wavy lines indicate the points of attachment to M,
each [symbol] represents a single or a double bond, provided that each cycle A, B, C and D is a heteroaromatic cycle,
T₁ is NRₐ₁ or CRₐ₁, T₂ is NRₐ₂ or CRₐ₂, T₃ is NRₐ₃ or CRₐ₃, T₄ is NRₐ₄ or CRₐ₄, T₇ is NRₐ₇ or CRₐ₇, T₈ is NRₐ₈ or CRₐ₈, T₉ is NRₐ₉ or CRₐ₉ and T₁₀ is NRₐ₁₀ or CRₐ₁₀, provided that when T₁ is NRₐ₁, then T₂ is CRₐ₂, when T₃ is NRₐ₃, then T₄ is CRₐ₄, when T₇ is NRₐ₇, then T₈ is CRₐ₈ and when T₉ is NRₐ₉, then T₁₀ is CRₐ₁₀,
Z₁ is N or CR_{b1}, Z₂ is N or CR_{b2}, Z₃ is N or CR_{b3}, Z₄ is N or CR_{b4}, Z₅ is N or CR_{b5}, Z₆ is N or CR_{b6}, Z₉ is N or CR_{b9}, Z₁₀ is N or CR_{b10}, Z₁ is N or CR_{b11}, Z₁₂ is N or CR_{b12}, Z₁₃ is N or CR_{b13} and Z₁₄ is N or CR_{b14}, provided that at least two of Z₁ to Z₃ and at least two of Z₄ to Z₆ and at least two of Z₉ to Z₁₁ and at least two of Z₁₂ to Z₁₄ are not N,
Rₐ₁ to Rₐ₁₂ and R_{b1} to R_{b16} each independently represent H, halogen, optionally substituted C₁-C₆ alkyl, optionally substituted C₂-C₆ alkenyl, optionally substituted C₂-C₆ alkynyl, optionally substituted carbocycle, optionally substituted aryl, optionally substituted heteroaryl, optionally substituted heterocycle, CN, NO₂, N₃, COR¹⁸, OR¹⁹ or NR²⁰R²¹,
or Z₃ and Z₄ in formula (b) are linked together so that LIG₂ represents: 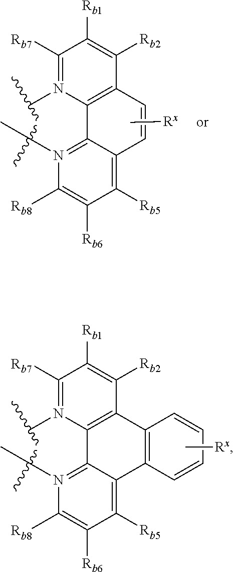
and/or
Z₁₁ and Z₁₂ are linked in formula (d) together so that LIG₃ represents: 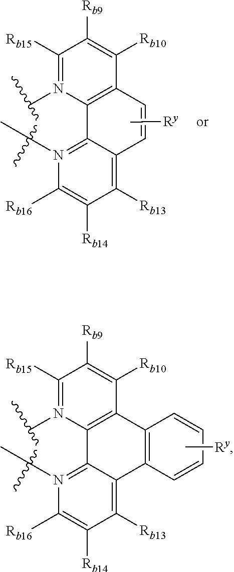
wherein R^{x} and R^{y} each independently represent one or several substituents selected in the group consisting of H, halogen, optionally substituted C₁-C₆ alkyl, optionally substituted C₂-C₆ alkenyl, optionally substituted C₂-C₆ alkynyl, optionally substituted carbocycle, optionally substituted aryl, optionally substituted heteroaryl, optionally substituted heterocycle, CN, NO₂, N₃, COR¹⁸, OR¹⁹ and NR²⁰R²¹,
R¹⁸ is selected in the group consisting of H, optionally substituted C₁-C₆ alkyl, OR²² and NR²³R²⁴
R¹⁹ to R²⁴ are each independently selected in the group consisting of H, optionally substituted C₁-C₆ alkyl and optionally substituted CO—(C₁-C₆ alkyl),
X^{m-} is a pharmaceutically acceptable anion,
m and n are independently 1, 2, 3 or 4, wherein n is 1 when M is rhenium, n is 2 when M is ruthenium or osmium, n is 3 when M is rhodium or iridium and n is 4 when M is platinum, and
y1 is 1, 2 or 3, y2 and y3 are independently 0, 1 or 2, provided that y1+y2+y3 is 3,
with the proviso that said compound is not:

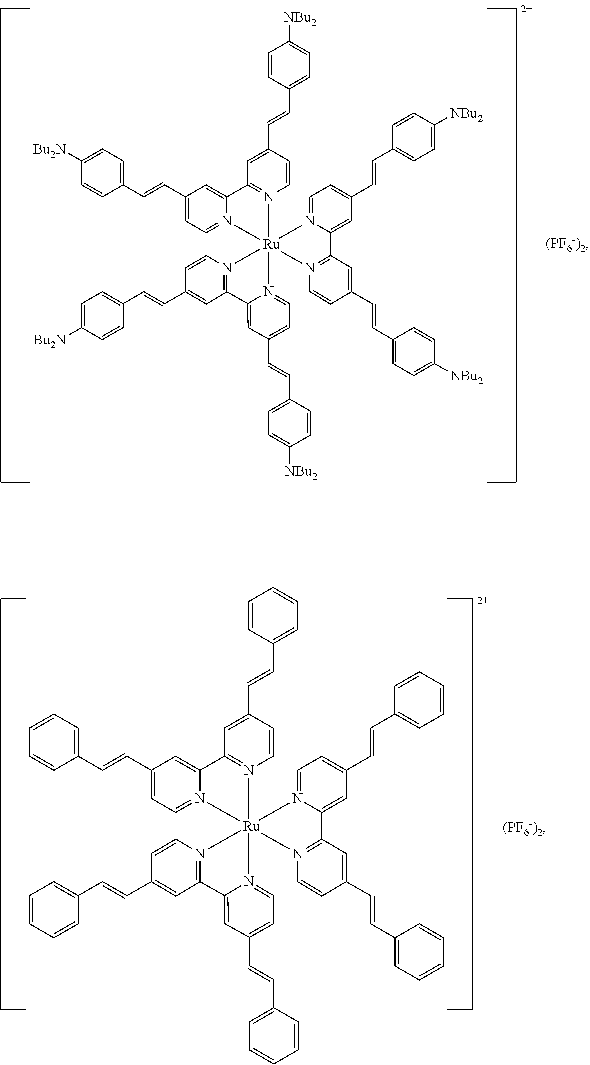

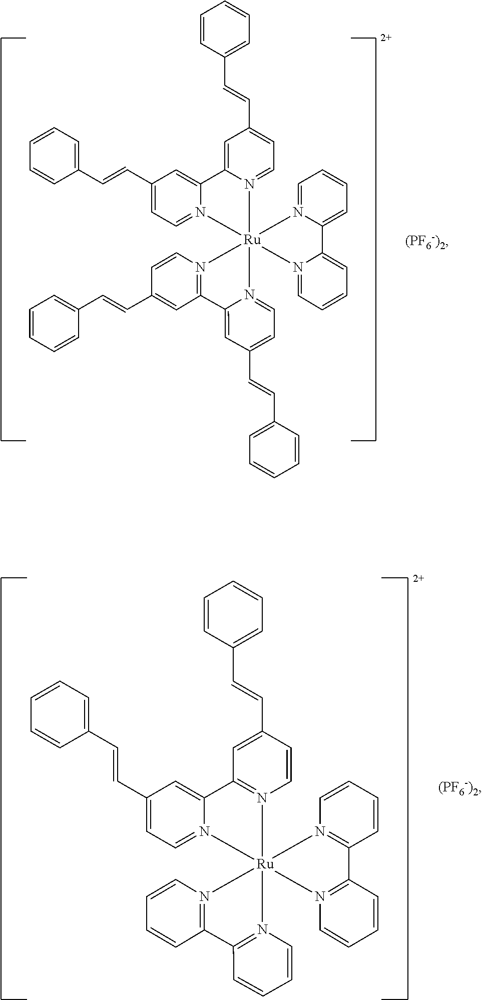

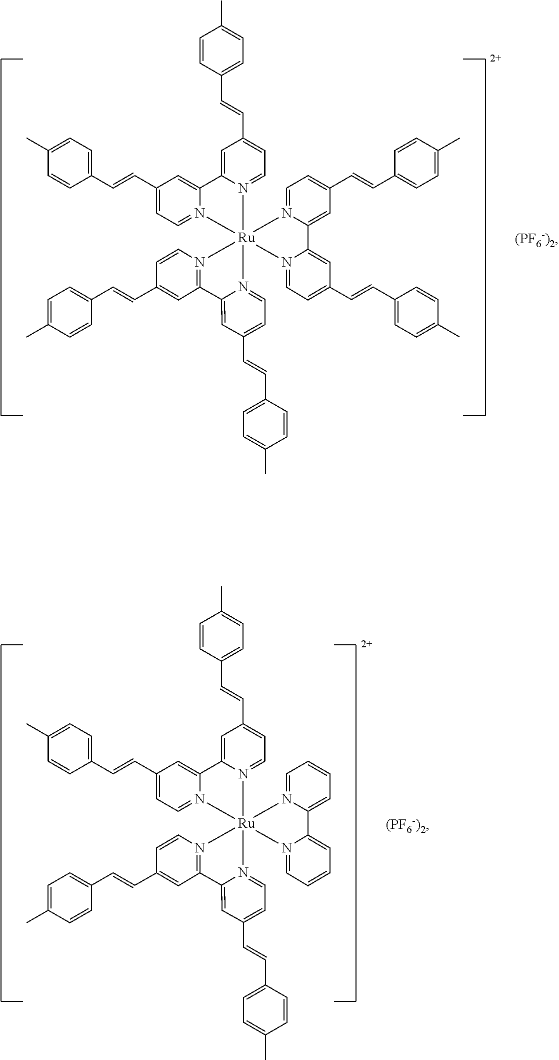

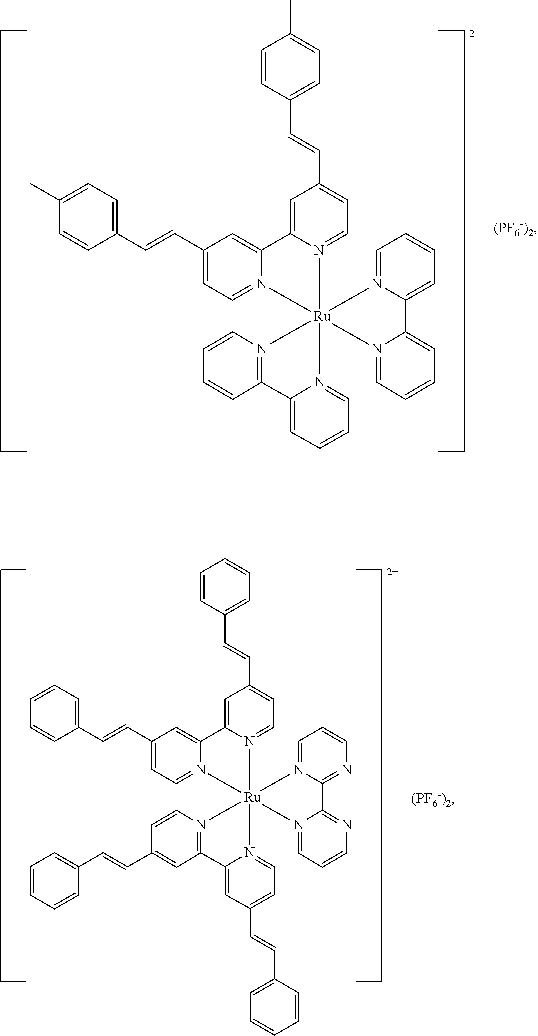

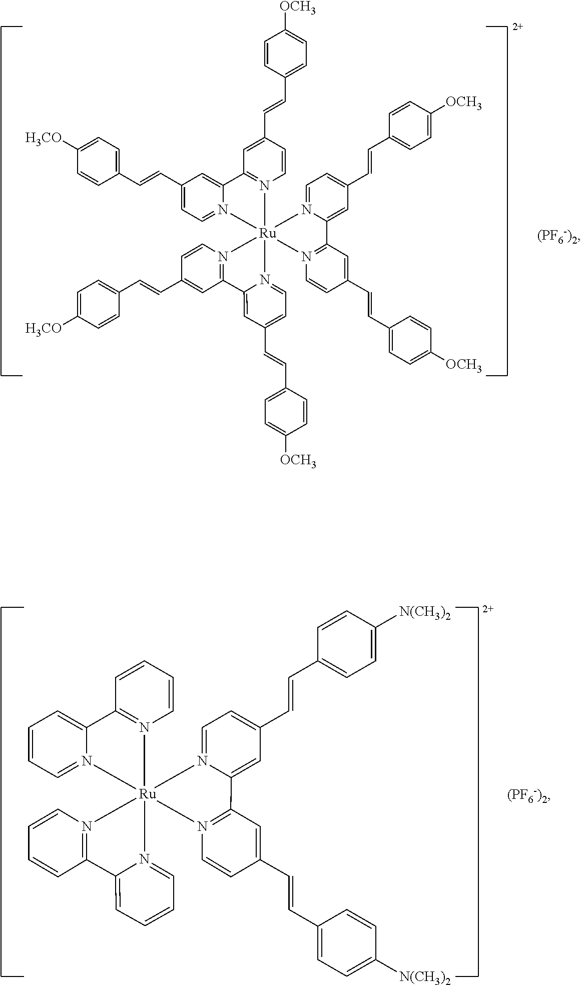

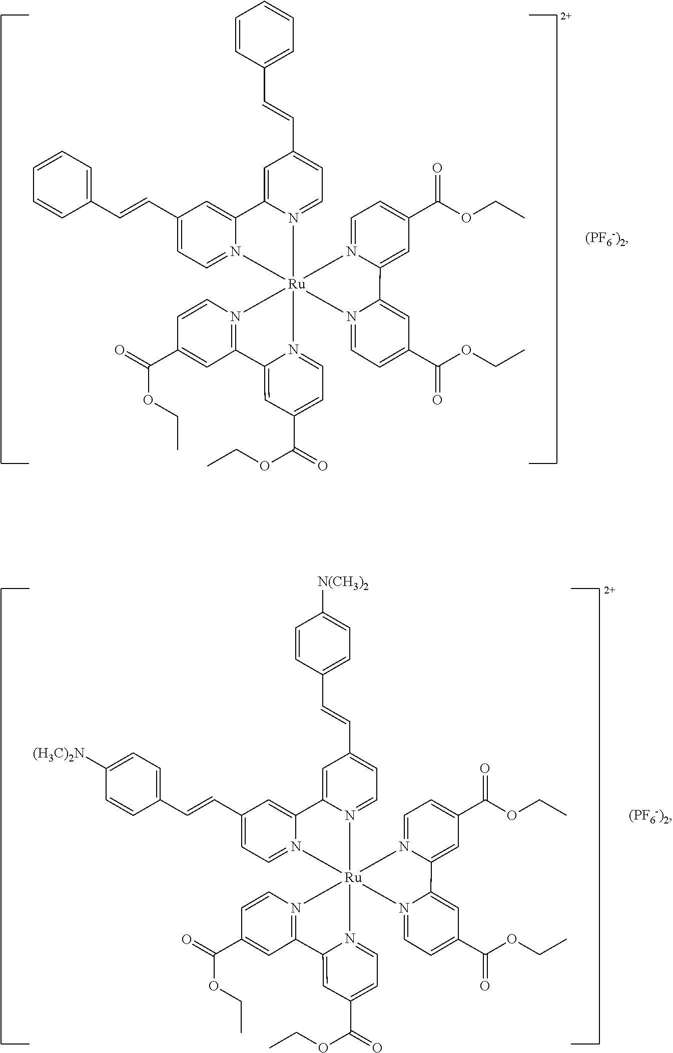

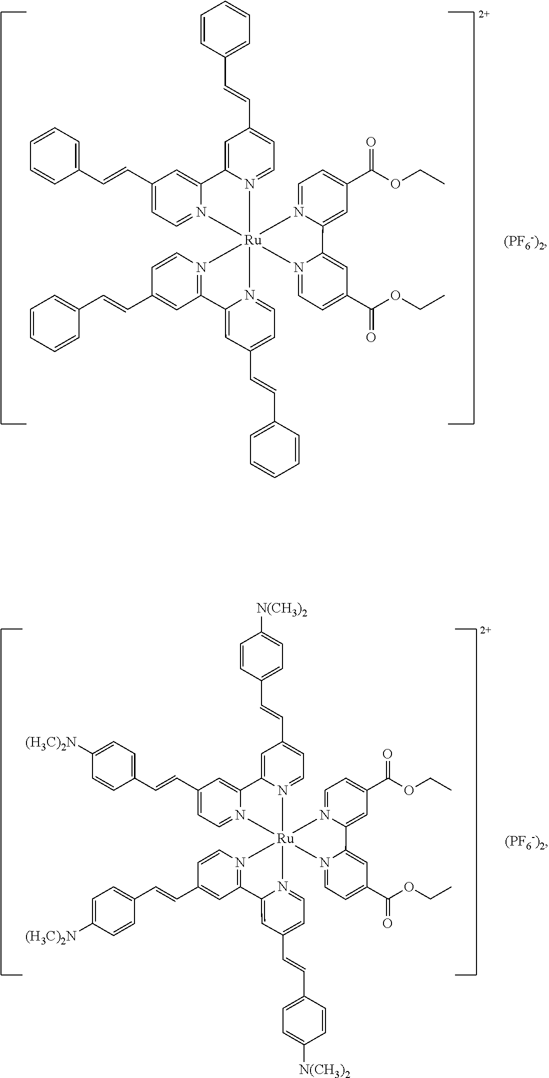

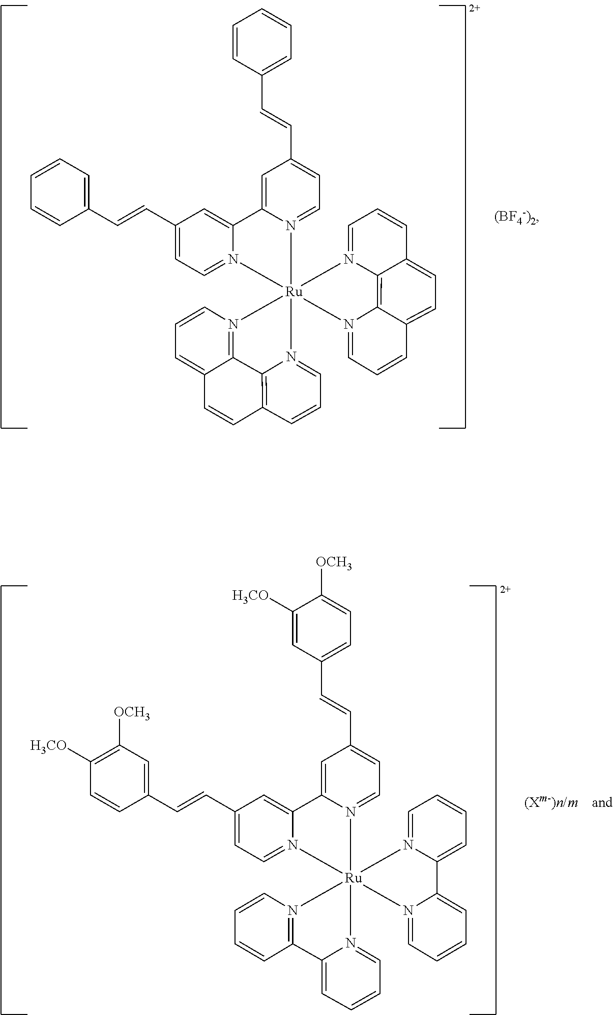

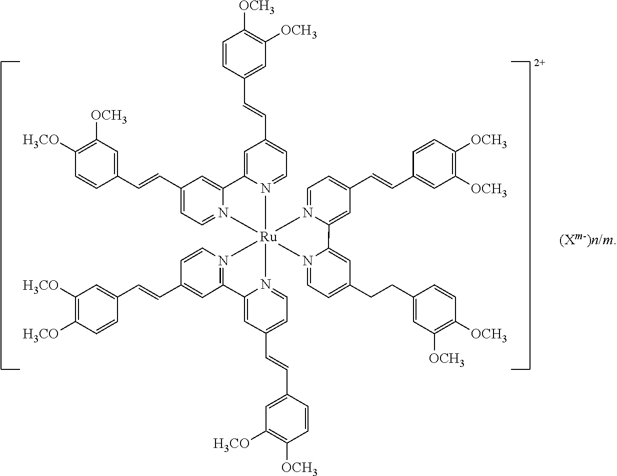

34. The compound according to claim 33, wherein R¹ and R² are one or several substituents each independently selected from the group consisting of H, halogen, OR⁹ and NR¹⁰R¹¹ and/or
Rₐ₁ to Rₐ₁₂ and R_{b1} to R_{b16} each independently represent H, halogen, C₁-C₆ alkyl, aryl, OR¹⁹ or NR²⁰R²¹, and
R^{x} and R^{y} each independently represent one or several substituents selected in the group consisting of H, halogen, C₁-C₆ alkyl, aryl, OR¹⁹ and NR²⁰R²¹,
with R¹⁹ to R²¹ being each independently selected in the group consisting of H and C₁-C₆ alkyl and/or y1 is 1, 2 or 3, y2 is 2, 1 or 0 respectively and y3 is 0 and/or M is ruthenium or osmium.

35. The compound according to claim 34, being selected from the group consisting of: 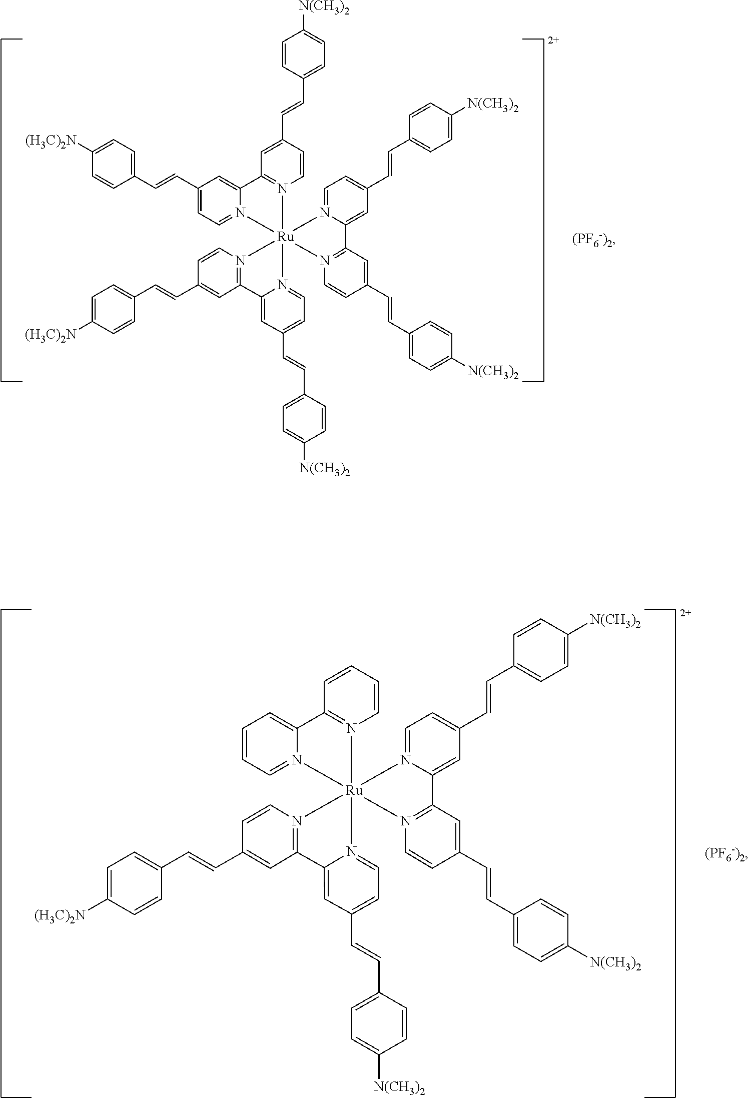 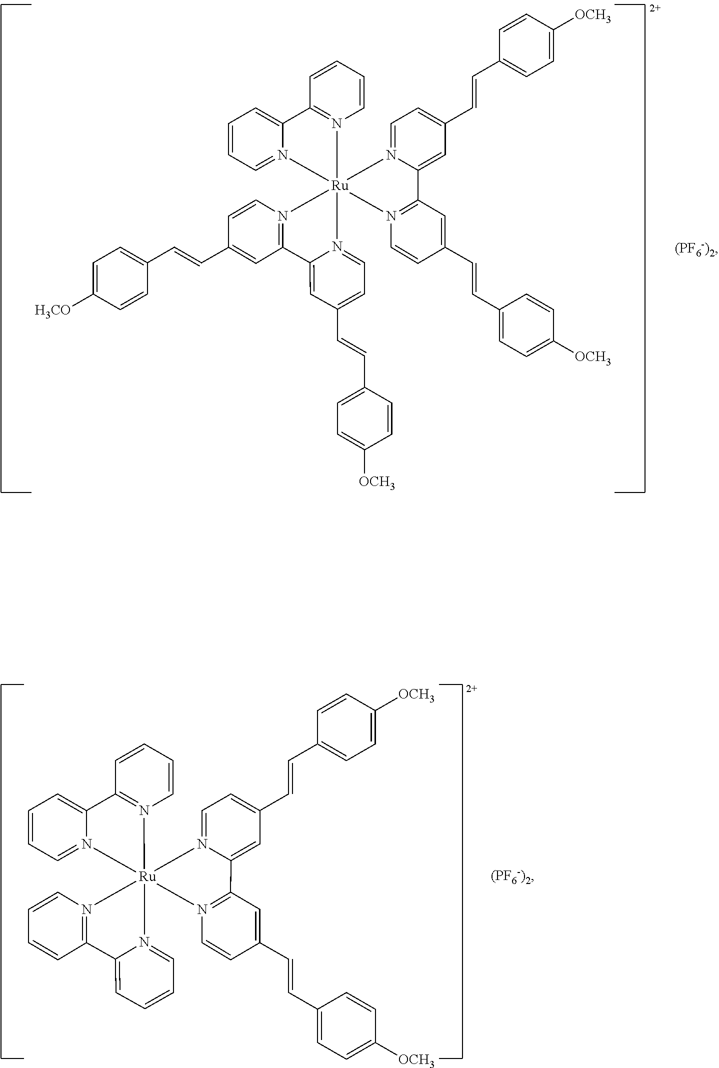 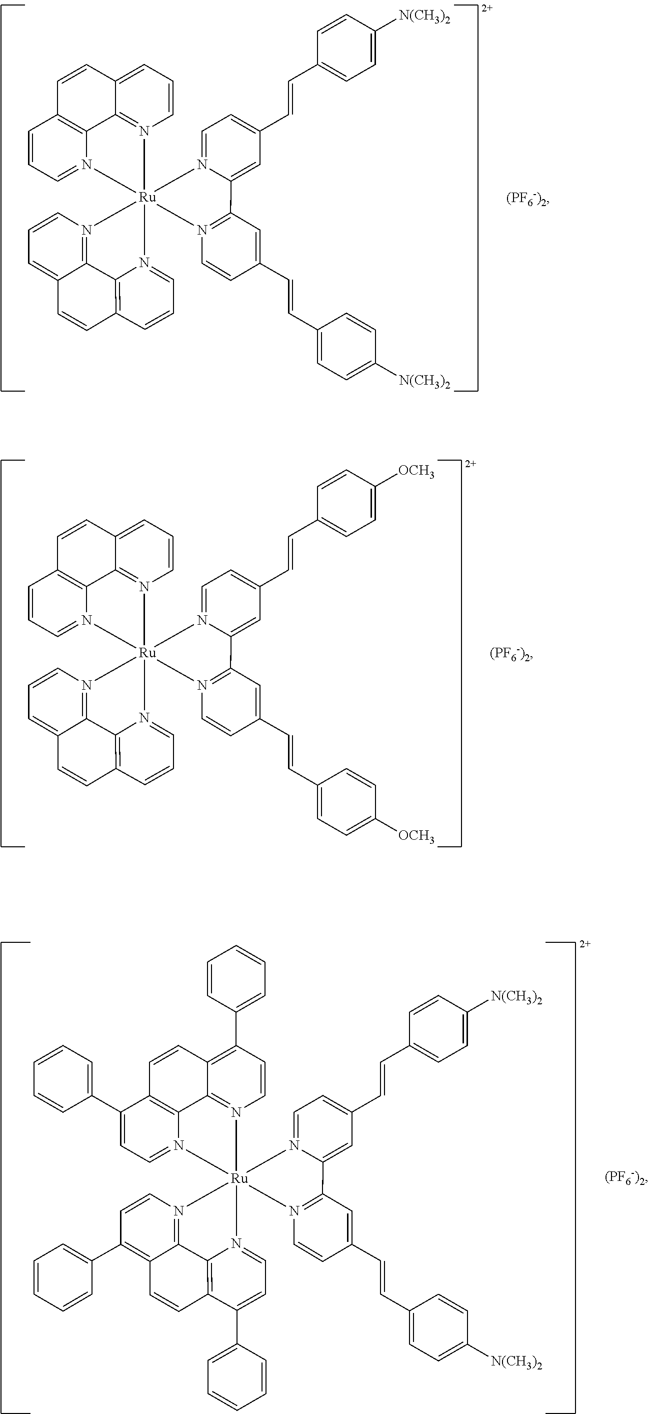 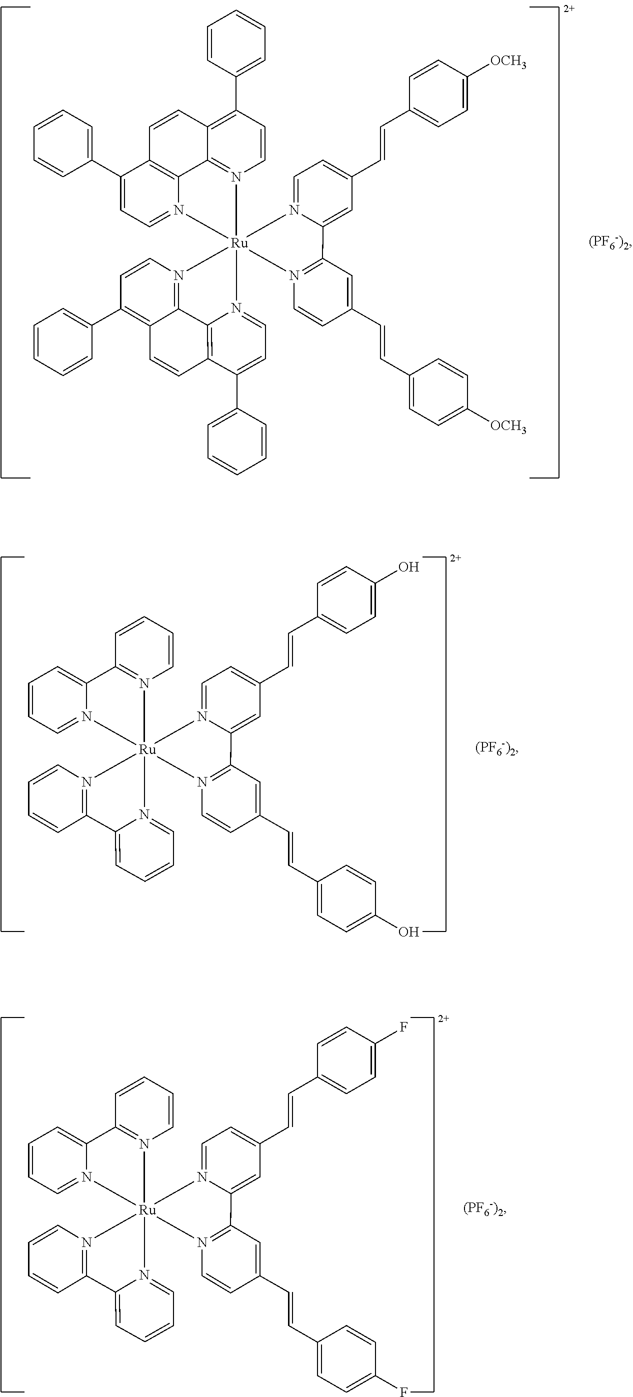 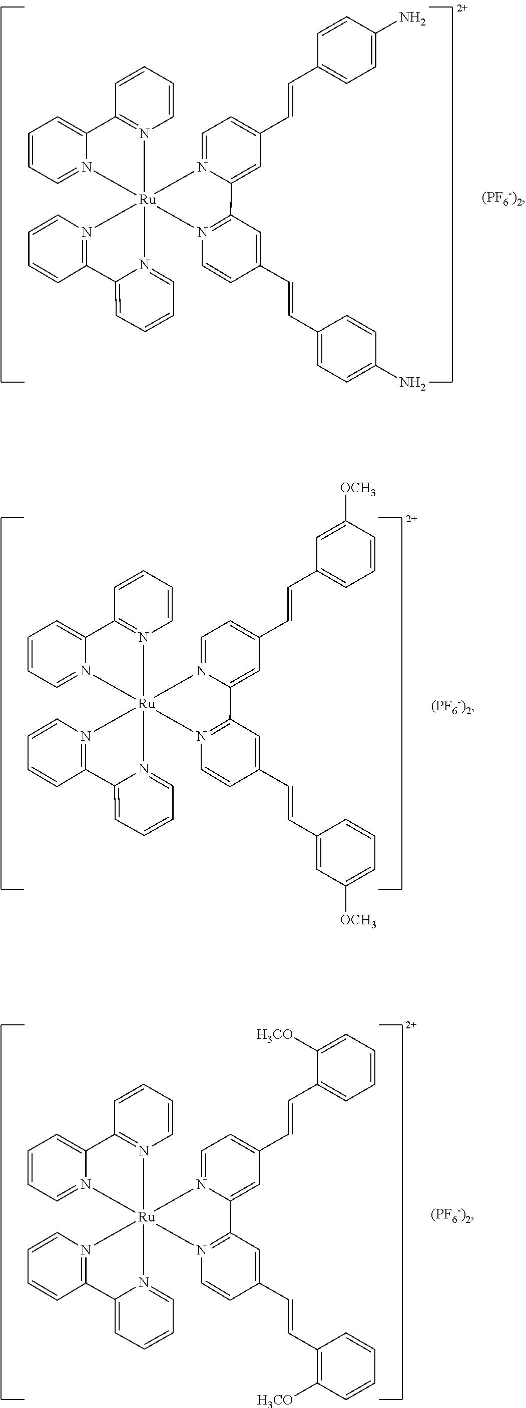 [chemical structure]
